(19) United States
(12) Patent Application Publication
(10) Pub. No.: US 2025/0034524 A1
(43) Pub. Date: Jan. 30, 2025

(54) HUMAN CORTICAL ORGANOIDS WITH ENGINEERED MICROGLIA-LIKE CELLS

(71) Applicant: Yale University, New Haven, CT (US)

(72) Inventors: In-Hyun Park, New Haven, CT (US); Bilal Cakir, New Haven, CT (US); Yoshiaki Tanaka, New Haven, CT (US)

(21) Appl. No.: 18/715,012

(22) PCT Filed: Dec. 1, 2022
(86) PCT No.: PCT/US2022/080739

§ 371 Date: May 30, 2024

Related U.S. Application Data
(60) Provisional application No. 63/284,872, filed on Dec. 1, 2021.

(51) Int. Cl. C12N 5/079; G01N 33/50

(52) U.S. Cl. CPC C12N 5/0622; G01N 33/5058; C12N 2500/32; C12N 2500/38; C12N 2500/44; C12N 2501/72; C12N 2503/02; C12N 2506/02; C12N 2513/00

(57) ABSTRACT

The invention provides microglial cell comprising cortical organoid cultures, methods of generating the same and methods of use thereof for identifying therapeutic agents.

### CROSS REFERENCE TO RELATED APPLICATIONS

This application claims priority to U.S. Provisional Application No. 63/284,872, filed Dec. 1, 2021, which is hereby incorporated by reference herein in its entirety.

### STATEMENT REGARDING FEDERALLY SPONSORED RESEARCH OR DEVELOPMENT

This invention was made with government support under MH118554 awarded by National Institutes of Health. The government has certain rights in the invention.

### BACKGROUND OF THE INVENTION

Microglia are the resident macrophages in the central nervous system (CNS). Their primary functions are to perform immune surveillance in the brain and facilitate brain network formation by regulating neuronal survival, synapse formation, elimination of apoptotic cells, and programmed cell death (Paolicelli et al., 2011, Science, 333:1456-1458; Schafer et al. 2012, Neuron, 74:691-705; Parkhurst et al., 2013, Cell, 155:1596-1609). Recent studies have demonstrated a critical link between impaired microglial function and neurodevelopmental (i.e., Autism or Schizophrenia) and neurodegenerative (i.e., Alzheimer's disease, AD) disorders (Salter et al., 2017, Nat Med, 23:1018-1027; Voineagu et al., 2011, Nature, 474:380-384; Wang et al., 2015, Cell, 160:1061-1071). Therefore, to investigate how human microglia functions in these diseases, it is essential to develop a model system that reproduces microglia behavior in the human brain.

3D brain organoid technology presents an innovative platform to investigate how the human brain develops and experiences neurological diseases (Arlotta et al., 2018, Nat Methods, 15:27-29). Brain organoids are made either by guided (Eiraku et al., 2008, Cell Stem Cell, 3:519-532; Pasca et al., 2015, Nat Methods, 12:671-678) methods using small molecules or by unguided (Lancaster et al., 2013, Nature, 501:373-379) protocols based on the intrinsic neuroectodermal differentiation of pluripotent stem cells (PSCs). The majority of brain organoids derived by the directed neuroectoderm differentiation do not contain the mesodermal lineage cells, such as myeloid microglia or endothelial cells (Velasco et al., 2019, Nature, 570:523-527; Tanaka et al., 2020, Cell Rep, 30:1682-1689 e1683; Quadrato et al., 2017, Nature, 545:48-53). Thus, these cells were directly differentiated from hESCs and co-cultured with brain organoids to study their functions (Lin et al., 2018, Neuron, 98:1294). Recently, Ormel et al. reported microglia within the organoids generated through the modified unguided protocol (Ormel et al., 2018, Nat Commun, 9:4167). However, the distribution and the number of functional microglia in these organoids are highly variable due to spontaneous and stochastic features of unguided differentiation. Overall, it is imperative to produce microglia in brain organoids in a highly reproducible manner to understand the function of these cells in the human brain and to dissect their role in the disease state.

Thus, there is a need in the art for improved model systems for reproducing microglia behavior in the human brain. The present invention satisfies this unmet need.

### SUMMARY OF THE INVENTION

In one embodiment, the invention relates to a method for preparing a microglial cell comprising cortical organoid culture, wherein the method comprises: a) co-culturing human epithelial stem cells containing an inducible Pu.1 transcription factor gene (Pu.1-hESCs) with human epithelial stem cells without the inducible Pu.1 transcription factor gene (hESCs) in neural induction media for at least 8 days; b) transferring the cells to a spinning human cortical organoid culture medium lacking vitamin A for at least 4 days; c) transferring the cells to a human cortical organoid culture medium with vitamin A; and d) inducing Pu.1 expression.

In one embodiment, the Pu.1-hESCs and hESCs are co-cultured at a ratio of 1:9. In one embodiment, the Pu.1-hESCs and hESCs are co-cultured at a ratio of 1:3. In one embodiment, the Pu.1-hESCs and hESCs are co-cultured at a ratio of 1:1.

In one embodiment, the neural induction media comprises DMEM-F12 with 15% (v/v) KSR, 5% (v/v) heat-inactivated FBS, 1% (v/v) Glutamax, 1% (v/v) MEM-NEAA, 100 μM β-Mercaptoethanol; supplemented with 10 μM SB-431542, 100 nM LDN-193189, 2 μM XAV-939 and 50 μM Y27632.

In one embodiment, the Pu.1 is inducible by doxycycline, and further wherein doxycycline is added to the neural induction media in a concentration of 0.5 μM dox beginning on day 2. In one embodiment, the FBS is removed from the neural induction medium beginning on day 2. In one embodiment, the Y27632 is removed from the neural induction medium beginning on day 4.

In one embodiment, the spinning human cortical organoid culture medium lacking vitamin A comprises a 1:1 mixture of DMEM-F12 and Neurobasal media, 0.5% (v/v) N2 supplement, 1% (v/v) B27 supplement without vitamin A, 0.5% (v/v) MEM-NEAA, 1% (v/v) Glutamax, 50 μM β-Mercaptoethanol, 1% (v/v) Penicillin/Streptomycin and 0.025% Insulin.

In one embodiment, the human cortical organoid culture medium with vitamin A comprises a 1:1 mixture of DMEM-F12 and Neurobasal media, 0.5% (v/v) N2 supplement, 1% (v/v) B27 supplement, 0.5% (v/v) MEM-NEAA, 1% (v/v) Glutamax, 50 μM β-Mercaptoethanol, 1% (v/v) Penicillin/Streptomycin and 0.025% Insulin.

In one embodiment, the Pu.1 is inducible by doxycycline, and further wherein doxycycline is added to the human cortical organoid culture medium with vitamin A in a concentration of 2 μM dox.

In one embodiment, the invention relates to a microglial cell comprising cortical organoid culture obtained by the method comprising the steps of: a) co-culturing human epithelial stem cells containing an inducible Pu.1 transcription factor gene (Pu.1ⁱ-hESCs) with human epithelial stem cells without the inducible Pu.1 transcription factor gene (hESCs) in neural induction media for at least 8 days; b) transferring the cells to a spinning human cortical organoid culture medium lacking vitamin A for at least 4 days; c) transferring the cells to a human cortical organoid culture medium with vitamin A; and d) inducing Pu.1 expression.

In one embodiment, the invention relates to an assay system comprising a microglial cell comprising cortical organoid culture obtained by the method comprising the steps of: a) co-culturing human epithelial stem cells containing an inducible Pu.1 transcription factor gene (Pu.1-hESCs) with human epithelial stem cells without the inducible Pu.1 transcription factor gene (hESCs) in neural induction media for at least 8 days; b) transferring the cells to a spinning human cortical organoid culture medium lacking vitamin A for at least 4 days; c) transferring the cells to a human cortical organoid culture medium with vitamin A; and d) inducing Pu.1 expression.

In one embodiment, the invention relates to a method of assaying a target agent comprising contacting a microglial cell comprising cortical organoid culture obtained by the method comprising the steps of: a) co-culturing human epithelial stem cells containing an inducible Pu.1 transcription factor gene (Pu.1-hESCs) with human epithelial stem cells without the inducible Pu.1 transcription factor gene (hESCs) in neural induction media for at least 8 days; b) transferring the cells to a spinning human cortical organoid culture medium lacking vitamin A for at least 4 days; c) transferring the cells to a human cortical organoid culture medium with vitamin A; and d) inducing Pu.1 expression with the target agent.

In one embodiment, the invention relates to a method for identifying a therapeutic agent, wherein the method comprises: contacting a microglial cell comprising cortical organoid culture obtained by the method comprising the steps of: a) co-culturing human epithelial stem cells containing an inducible Pu.1 transcription factor gene (Pu.1ⁱ-hESCs) with human epithelial stem cells without the inducible Pu.1 transcription factor gene (hESCs) in neural induction media for at least 8 days; b) transferring the cells to a spinning human cortical organoid culture medium lacking vitamin A for at least 4 days; c) transferring the cells to a human cortical organoid culture medium with vitamin A; and d) inducing Pu.1 expression with one or more candidate agents, detecting the presence or absence of one or more change in the microglial cell comprising cortical organoid culture that is indicative of therapeutic efficacy, and identifying the candidate agent as a therapeutic agent if the presence or absence of one or more of said changes in the microglial cell comprising cortical organoid culture is detected.

In one embodiment, the change in the microglial cell comprising cortical organoid co-culture is a change in cell viability, organoid size, morphology, quantification of epithelial subsets, cell proliferation, transcriptome, protein levels or post-translational modifications of proteins, metabolism, production of soluble factors and any combination thereof of the microglial cell comprising cortical organoid cells as compared to a comparator control.

In one embodiment, the therapeutic agent is suitable for the treatment of a neurodevelopmental or neurodegenerative disease or disorder. In one embodiment, the disease or disorder is autism, schizophrenia, Alzheimer's disease (AD) or another dementia, Parkinson's disease (PD) or a PD-related disorder, frontotemporal dementia (FTD), amyotrophic lateral sclerosis (ALS), Huntington's disease (HD), spinocerebellar ataxias (SCAs), prion disease, spinal muscular atrophy (SMA), lewy body dementia (LBD), multisystem atrophy, primary progressive aphasia, multiple sclerosis (MS), ischemic stroke, traumatic brain injury, HIV-associated dementia, or another neurodegenerative, neurological or psychiatric disease or disorder.

In one embodiment, the invention relates to a kit comprising at least one component for use in a method of preparing a microglial cell comprising cortical organoid culture, wherein the method comprises: a) co-culturing human epithelial stem cells containing an inducible Pu.1 transcription factor gene (Pu.1ⁱ-hESCs) with human epithelial stem cells without the inducible Pu.1 transcription factor gene (hESCs) in neural induction media for at least 8 days; b) transferring the cells to a spinning human cortical organoid culture medium lacking vitamin A for at least 4 days; c) transferring the cells to a human cortical organoid culture medium with vitamin A; and d) inducing Pu.1 expression. In one embodiment, the kit comprises Pu.1ⁱ-hESCs.

In one embodiment, the invention relates to a kit comprising at least one microglial cell comprising cortical organoid culture.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1F depict exemplary experimental results demonstrating the generation of cortical organoids with microglia-like cells via induction of PU.1 expression. FIG. 1A depicts (left) a construct of inducible PU.1/eGFP to generate lentivirus; (right) a construct to target the AAVS1 locus to generate rtTA expressing hESC line (BC4). FIG. 1B depicts PU.1 (MOI 4) infection under the embryonic body (EB) or neuron differentiation media for five days. FIG. 1C depicts the expression of microglia-related genes under EB (top) and neuron (bottom) differentiation conditions were measured relative to control hESCs and normalized to β-Actin. Data represent the mean±SEM (n=3). FIG. 1D depicts immunostaining for microglia marker IBA1 in hESCs differentiated without or with PU.1 induction in EB, or neuron differentiation media. FIG. 1E depicts (left) representative images are demonstrating GFP+ cells expressing PU.1 that form amoeboid-like structures; (right) quantification of GFP+ signal intensity per mm3 in mhCOs at days 30 and 70. Z stack confocal imaging was performed (˜40-50 μm). Data represent the mean±SEM (n=5, from three independent batches). Unpaired two-tail t-test was used for comparison (***p<0.0001). FIG. 1F depicts (left) co-immunostaining for GFP and IBA1 in mhCOs at day 70; (right) quantification of co-expression of GFP and IBA1 cells. Data represent the mean SEM (n=5, from three independent batches). Bottom, Pearson's correlation coefficient of IBA1 with GFP in mhCOs at day 70. The scale bar represents 50 μm in FIG. 1D, FIG. 1E, and FIG. 1F.

FIG. 2A through FIG. 2H depict exemplary experimental results demonstrating the characterization of microglia-like cells in mhCOs. FIG. 2A depicts a schematic for generating mhCOs. 10% of PU.1 infected hESCs were mixed with 90% parental HES3 hESCs, and PU.1 priming and full induction were performed on day 2 and 18, respectively. FIG. 2B depicts the expression of microglia-related genes from control hCOs and mhCOs (30- and 70-day old). Gene expression was measured relative to control organoids on day 30 and normalized to β-Actin. Data represent the mean±SEM (n=5, from three independent batches). FIG. 2C depicts (left) immunostaining for IBA1 reveals the production of microglia-like cells in sectioned-mhCOs at days 30 and 70. IBA1⁺ cells were not found in control hCOs; (right) a sholl analysis of IBA1+ microglia-like cells from mhCOs at different time points. Data represent the mean±SEM (n=5, from three independent batches). FIG. 2D and FIG. 2E depict immunostaining of mhCOs at day 70 and isolated microglia co-cultured with neurons (2D) for IBA1 and CSF1R (FIG. 2D) TMEM119 and P2RY12 (FIG. 2E). Representative images were shown (n=5, from two independent batches). FIG. 2F depicts (top) co-expression of PU.1 and IBA1 in hCOs and mhCOs at day 30, and 70; (bottom) quantification of Pu.1 derived IBA1 microglia-like cells. Data represent the mean±SEM (n=5, from three independent batches). Bottom, Pearson's correlation coefficient of IBA1 with PU.1 in mhCOs at days 30 and 70. FIG. 2G depicts (top) co-immunostaining for Ki67 and IBA1 in mhCOs at day 70; (bottom) quantification of proliferating IBA1 microglia-like cells. Data represent the mean±SEM (n=5, from three independent batches). FIG. 2H depicts (left) high-resolution imaging showed microglia isolated from mhCOs at day 90 and co-cultured D90 cortical neurons for 3 days contained inclusions of PSD95; (right) quantification of PSD95 particles in IBA1+ microglia-like cells (n=8). The scale bar represents 50 μm in FIG. 2C, FIG. 2D, FIG. 2E, FIG. 2F, FIG. 2G, and 20 μm in FIG. 2H.

FIG. 3A through FIG. 3F depict exemplary experimental results demonstrating the effects of microglia-like cells in mhCOs and PU.1 induced microglia to engraft mouse brain. FIG. 3A depicts FISH images of organoid sections with NPAS4 and FOS probes. FIG. 3B depicts (left) representative images demonstrating calcium transient traces observed from individual neurons of control- and mhCOs (day 70-80), the single-cell tracings of calcium transient were recorded in control and mhCO organoids; (right) the average amplitude of ΔF/F per cell and firing frequency of neurons from hCOs and mhCOs (n=25). FIG. 3C depicts an SEM image showing the presence of microglia-like cells from mhCO, but not in hCO, at day 75 (2000× magnification). FIG. 3D illustrates (left) co-immunostaining for SOX2 and TBR2, and SOX2, CTIP2, and SATB2 in 35- and 70-day old control hCOs and mhCOs, respectively; (right) quantification of SOX2+, TBR2+, SATB+, and CTIP2+ cells ratio over DAPI+ cells within organoids. Data are representative images of 5 organoids from three independent experiments. FIG. 3E depicts transplantation of PU.1 derived microglia and Aβ_oligo in the immune-deficient mice brain. Green and pink circles, respectively, represent microglia and Aβ_oligo injection sites. FIG. 3F depicts co-immunostaining for human-specific CD68, and Aβ indicates that successfully engrafted PU.1 induced microglia phagocytoses injected Aβ_oligo. n=4 animals. The scale bar represents 100 μm in FIG. 3A, 10 μm in FIGS. 3B and 3C, and 50 μm in FIGS. 3D and 3F.

FIG. 4A through FIG. 4I depict exemplary experimental results demonstrating the single-cell map of mhCOs. FIG. 4A depicts a Uniform Manifold Approximation and Projection (UMAP) plot of single cells from hCOs and mhCOs colored by cell type assignment (left) and organoid type (right). CN: cortical neuron, IN: interneuron, Neuron: non-committed neuron, Inter: intermediate, NPC: neuronal progenitor cell, AS: astrocyte, GPC: glia progenitor cells, BRC: BMP responsible cell, CBC: Cilia-bearing cell, UPRC: unfolded protein response-related cell, UN: unassigned cell, PGC: proteoglycan-expressing cell, MG: microglia, CEC: Claudin-enriched cell, KEC: Keratin-enriched cell. Data depicts results from 26962 cells. FIG. 4B depicts a pie chart representing the cell count from hCO and mhCO in MG, CEC, KEC, and PGC clusters. FIG. 4C illustrates a heatmap representing the upregulation of microglia and endothelial-related genes with PU.1 induction. Relative expression value in mhCO to hCO was normalized as Z-score. FIG. 4D through FIG. 4I depicts an integrative analyses of scRNA-seq of microglia from mhCOs and human fetal brains. FIG. 4D depicts trajectory plots demonstrating pseudotime (left) and age (right). FIG. 4E depicts trajectory plots separated by the human fetal brain at each developmental stage and organoid. The treatment of Aβ further separated organoid-derived microglia cells. FIG. 4F depicts gene expression profiles over pseudotimes for representative differentially-expressed genes. FIG. 4G depicts significant GO terms of genes elevated with pseudotimes. FIG. 4H depicts a histogram of pseudotime. Cells were categorized into four distinct stages with thresholds that are shown by dashed lines. FIG. 4I depicts a ratio of fetal brain ages across the developmental stages.

FIG. 5A through FIG. 5F depict exemplary experimental results demonstrating the cluster labeling of single-cell transcriptome analysis. FIG. 5A depicts a UMAP plot of single cells from non- and Aβ-treated hCOs and mhCOs colored by cell clusters. All 49,867 cells from non- and Aβ-treated hCOs and mhCOs are shown. FIG. 5B depicts the expression patterns of neuronal (STMN2, GAP43, and DCX) and early neurogenesis markers (SOX2, HES1, and VIM). Data depicts results from 49,867 cells from non- and Aβ-treated hCOs and mhCOs. FIG. 5C depicts a GSEA of gene signatures for neurons, NPC, endothelium, astrocyte, and microglia. Enrichment and depletion are scaled by −log 10(FDR) and shown by red and blue colors, respectively. Data depicts results from 49,867 cells from non- and Aβ-treated hCOs and mhCOs. FIG. 5D depicts a schematic representation of the cluster labeling method. ME: mesoderm. FIG. 5E depicts a comparison of average UMI, mitochondrial-derived reads, marker expression, and GO enrichment across 40 clusters. FIG. 5F depicts a plot of single cells from mhCOs and human brains in the shared UMAP space colored by cell type assignment (Bhaduri et al., 2020, Nature, 578:142-148).

FIG. 6A through FIG. 6F depict exemplary experimental results demonstrating the characterization of cell types in mhCOs using scRNAseq. FIG. 6A depicts a GSEA of gene signatures for MG, CEC, and KEC clusters between differentiated microglia (dMG) and microglia precursor (MP). FIG. 6B depicts a cell trajectory analysis of CEC, KEC, and MG clusters with human fetal microglia cells (Kracht et al., 2020, Science, 369:530-537). CEC and KEC correspond to cells at early gestation week (GW9-11) and earlier stage than MG cluster. FIG. 6C depicts a volcano plot representing differential gene expression between mhCO and hCO. 685 up- and 242 down-regulated genes are shown by turquoise and salmon color, respectively. The other 31,811 genes are indicated by gray color. FIG. 6D depicts a GO enrichment of differentially expressed genes in mhCO. FIG. 6E depicts a GSEA of gene signatures for organoid-grown microglia-like cell (oMG) by Ormel and his colleagues (Ormel et al., 2018, Nat Commun, 9:4167). Enrichment and depletion are scaled by −log 10(FDR) and shown as red and blue colors, respectively. FIG. 6F depicts an enrichment of gene signatures for primary microglia (pMG) and oMG by Ormel et al. (Ormel et al., 2018, Nat Commun, 9:4167) in the MG cells in mhCOs.

FIG. 7A through FIG. 7I depict exemplary experimental results demonstrating the microglia lineage commitment of GFP-expressing cells and their roles in the organoid (mhCO). FIG. 7A depicts a construct to target the AAVS1 locus to generate rtTA-PU.1-IRES-eGFP⁺ hESC line (BC61). FIG. 7B depicts a schematic showing protocol to generate mhCOs by using hESCs (BC61). FIG. 7C depicts time-lapse imaging indicating GFP⁺ microglia over 100 min within mhCOs. Representative images with white arrowheads show motile microglia. Time is shown in minutes. FIG. 7D depicts a FACS analysis of dissociated mhCOs at day 75 shows that GFP⁺ and GFP⁻ cells are sorted for performing scRNA-seq. FIG. 7E depicts a cell trajectory analysis of the single-cell transcriptome of GFP⁺ and GFP⁻ cells. Cells are colored by trajectory branches and library sources. FIG. 7E depicts a ratio of GFP⁺ and GFP⁻ cells in MG clusters. FIG. 7G depicts a comparison of GFP expression between MG and other branches (two-sided T-test p<2.2e-16). FIG. 7H depicts a relationship between GFP/PU1 expression and MG lineage commitment. (Top) GFP and PU.1 expression are plotted in the cell trajectory map. (Bottom) Correlation between GFP/PU.1 expression and efficiency of MG lineage commitment. Cells are sorted by GFP/PU.1 expression and divided into 100-cells bins. The number of cells in the MG branch is shown in each bin. FIG. 7I depicts (top) a schematic showing Aβ42-oligo-(HiLyte)-555 treatment on mhCOs; (bottom) a representative image is showing GFP+ microglia phagocytosing Aβ (red) in around 32 min within mhCOs. The scale bar represents 20 μm in C and 10 μm in FIG. 7I.

FIG. 8A through FIG. 8D depict exemplary experimental results demonstrating the single-cell transcriptome analysis of GFP+ and GFP population in the organoid. FIG. 8A illustrates a FACS analysis of dissociated hCOs at day 75 shows that GFP+ cells are not present in hCOs. FIG. 8B depicts a marker expression pattern across branches. FIG. 8C depicts the number of cells including GFP-derived reads in GFP+ and GFP− libraries. FIG. 8D depicts the number of cells co-expressing GFP and PU.1.

FIG. 9A through FIG. 9H depict exemplary experimental results demonstrating the effects of Aβ treatment on cortical organoids. FIG. 9A depicts the experimental design to test the effect of Aβ_oligo treatment on control hCOs and mhCOs. FIG. 9B depicts co-Immunostaining for CD68 and Aβ in Aβ-treated control hCOs, mhCOs, and the adult human brain. Data are representative images of 5 organoids from three independent experiments. The scale bar represents 50 μm. FIG. 9C depicts a UMAP plot of single cells from non- and Aβ-treated hCOs and mhCOs colored by cell type assignment (left) and organoid type (right). Data depicts results from 49,867 cells. FIG. 9D depicts a histogram representing significant gene induction related to microglia activation in non- and Aβ-treated mhCOs. p=2.51e-11 (AIF1), 3.64e-4 (CSF1R), 2.79e-7 (PTPRC), 2.04e-10 (C1QC), 9.52e-7 (LCP1) and 1.42e-11 (CTSS) with two-sided t-test. FIG. 9E depicts a volcano plot representing differential gene expression in microglia between non- and Aβ-treated mhCOs (left). 775 up- and 175 down-regulated genes are shown by purple and turquoise color, respectively. The other 31,788 genes are shown by gray color. The top five significant GO terms of upregulated genes in Aβ-treated microglia were shown by circle size (right). FIG. 9F depicts a heatmap representing the down-regulation of neuronal developmental genes in hCOs with Aβ treatment. Relative expression value in non-treated hCO to Aβ-treated hCOs was normalized as Z-score. FIG. 9G depicts a GO enrichment of global differentially-expressed genes between non- and Aβ-treated organoids. Left and right bar plots were obtained from hCO and mhCO, respectively. FIG. 9H depicts a GSEA of Braak stage-specific gene signatures in Aβ-treated hCOs and mhCOs. The enrichment and depletion were visualized by blue and green colors, subsequently.

FIG. 10A through FIG. 10C depict exemplary experimental results demonstrating the characterization of Ab treatment on cortical organoids. FIG. 10A depicts (left) representative images are showing the morphology of control hCOs and mhCOs with and without Aβ treatment. Right, quantification of % of surface damage on organoids with and without Aβ treatment. Data are representative images of 5 organoids from three independent experiments (***p<0.0001). FIG. 10B depicts (left) TUNEL staining of organoids after 90-day culture. Right, quantification of TUNEL+/DAPI+ cells in hCOs and mhCOs at days 90. Data represent the mean±SEM (n=12, from three independent batches). Unpaired two-tail t-test was used for comparison (***p<0.0001). FIG. 10C depicts representative immunostaining of IBA1, LAMP1, and Aβ in hCOs exposed for 72 hours to Ab_oligo (n=5, from three independent batches). The scale bar represents 1 mm in FIG. 10A, 50 μm in FIG. 10B, and 5 μm in FIG. 10C.

FIG. 11A through FIG. 11G depict exemplary experimental results demonstrating the characterization of Ab treatment on cortical organoids. FIG. 11A depicts a GO enrichment of upregulated genes in microglia clusters from AD patient-derived brain compared to healthy donor brain. FIG. 11B depicts an expression of dendrite and synaptic-related genes from control hCOs and mhCOs with and without Aβ treatment. Gene expression was measured relative to control organoids without Aβ treatment and normalized to β-Actin. Data represent the mean±SEM (n=3, from three independent batches). FIG. 11C depicts (left) immunostaining of control hCOs and mhCOs with and without Aβ treatment for NRXN1 and MAP2; (right) quantification of NRXN1 clustering per DAPI and MAP2 signal intensity per area from organoids with and without Aβ treatment (n=10, ***p<0.0001, from three independent batches). FIG. 11D depicts a differential expression of neuronal and glial differentiation genes with Aβ treatment. The upper and lower panel represents hCO and mhCO datasets, respectively. Differential expression level (−log 10(p-value)) was visualized with circle size. Up and down regulation was scaled from red to blue colors. FIG. 11E depicts the differential expression of ferroptosis-related genes with Aβ treatment. FIG. 11F depicts differential expression of neuronal and glial differentiation genes with Aβ treatment. Upper and lower panels represent hCO and mhCO datasets, respectively. Differential expression level (−log 10(p-value)) was visualized with circle size. Up- and down-regulation was scaled from red to blue colors. FIG. 11G depicts differential expression of ferroptosis-related genes with Aβ treatment.

FIG. 12A through FIG. 12E depict exemplary experimental results demonstrating the CRISPRi-mediated suppression of AD-genes in MG from mhCOs. FIG. 12A depicts the relationship between target genes and pathways for endocytosis, phagocytosis, and degradation. FIG. 12B depicts the differential gene expression between non- and Aβ-treated mhCOs in each knockdown. FIG. 12C depicts (left) whole-mount immunostaining for CD11b in hCO, mhCO, and mhCOs with CRISPRi-mediated suppression of TREM2, SORL1, or CD33 at day 60; (right) quantification of the number of CD11b+ microglia-like cells/μm³. The scale bar represents 100 μm. FIG. 12D depicts (left) representative images of control hCO, mhCO, and mhCOs with CRISPRi-mediated suppression of TREM2, SORL1, or CD33 at 60-day old with and without Aβ treatment, white arrowheads are showing damaged surfaces; (middle) schematic showing quantification of surface damages induced by Aβ treatment; (right) quantification of % of surface damage on organoids with and without Aβ treatment. Data are representative images of 5 organoids from three independent experiments. The scale bar represents 0.5 mm. FIG. 12E depicts (left) cleaved caspase-3 staining of organoids after 60-day culture with and without Aβ treatment; (right) quantification of cleaved caspase-3+/DAPI+ cells in hCOs and mhCOs at days 60 with and without Aβ treatment. Data represent the mean±SEM (n=6, from two independent batches). The scale bar represents 50 μm.

FIG. 13A through FIG. 13F depict exemplary experimental results demonstrating the generation of mhCOs expressing knockdown of AD-associated microglia genes. FIG. 13A depicts a UMAP plot of CROP-seq from non-, and Aβ-treated mhCOs are colored by cell types. The expression pattern of endocytosis-related genes across cell types is shown by red color. FIG. 13B depicts a qPCR analysis of HES3 hESCs containing 2 different gRNAs against the AD-associated microglia genes demonstrated different knockdown efficiency after 6 induction days. UMAP plot of single cells from non- and Aβ-treated hCOs colored by cell type assignment (left) and organoid type (right). Gene expression was measured relative to un-induced HES3 hESCs and normalized to β-Actin. Data represent the mean±SEM (n=3). FIG. 13C provides a depiction of target genes to knock down from mhCOs. FIG. 13D provides a schematic of the method for generating mhCOs with an AD-associated microglia gene knockdown. Timeline and lentivirus bearing gRNAs of target genes used for cortical organoids are shown. FIG. 13E depicts the expression of microglia-related genes and target genes from mhCO variants at day 57 was measured relative to control hCOs. Data represent the mean±SEM (n=5, from three independent batches). FIG. 13F depicts (left) a TUNEL staining of organoids after 60-day culture with and without Aβ treatment; (right) quantification of TUNEL+/DAPI+ cells in hCOs and mhCOs at days 60 with and without Aβ treatment. Data represent the mean±SEM (n=7, from three independent batches). The dash lines indicate the surface of the samples. The scale bar represents 100 μm.

FIG. 14A and FIG. 14B depict exemplary experimental results demonstrating the cholesterol turnover in mhCO variants. FIG. 14A depicts (left) CYP46A1 staining of organoids after 60-day culture with and without Aβ treatment. Right, quantification of CYP46A1+ signal intensity per mm2 in hCOs and mhCOs at days 60 with and without Aβ treatment. Data represent the mean±SEM (n=8, from three independent batches). The scale bar represents 50 μm. FIG. 14B depicts the total cholesterol (left), free cholesterol (middle), and cholesteryl esters concentrations (right, g/mg tissue) in the whole brain organoid variants with and without Ab treatment. Data represent the mean±SEM (n=5, **p<0.01, ***p<0.001, from three independent batches).

### DETAILED DESCRIPTION

In one embodiment, the invention relates to ex vivo culture platforms and methods of using said ex vivo culture systems, wherein the ex vivo culture platform comprises human cerebral organoids comprising microglia-like cells (mhCOs).

In one embodiment, the invention is based, in part, on the development of an ex vivo platform to evaluate individual-specific responses to agents of interest, such as neuroprotective agents and potential therapeutic agents. For example, in one embodiment, the invention provides an innovative platform to investigate how the human brain develops and experiences neurological diseases. In one embodiment, the ex vivo platform comprises microglia containing cortical organoids for use in evaluating agents for treatment of a neurodevelopmental or neurodegenerative disease or disorder. In one embodiment, the ex vivo platform comprises microglia containing cortical organoids for use in evaluating agents for the treatment or prevention of autism, schizophrenia, Alzheimer's disease (AD) and other dementias, Parkinson's disease (PD) and PD-related disorders, frontotemporal dementia (FTD), amyotrophic lateral sclerosis (ALS), Huntington's disease (HD), spinocerebellar ataxias (SCAs), prion disease, spinal muscular atrophy (SMA), lewy body dementia (LBD), multisystem atrophy, primary progressive aphasia, multiple sclerosis (MS), ischemic stroke, traumatic brain injury, HIV-associated dementia, and other neurodegenerative, neurological and psychiatric diseases or disorders.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Co-culture” refers to two or more cell types maintained together in the same culture chamber, such as a dish, tube, container, or the like. In some embodiments, the two or more cell types are maintained in conditions suitable for their mutual function or in conditions for their mutual interaction. In the context of the present disclosure, an “organoid co-culture” relates to an epithelial organoid, as defined elsewhere, in culture with a non-epithelial cell type, specifically an immune cell type. In some embodiments, cell types in co-culture exhibit a structural, biochemical and/or phenomenological association that they do not exhibit in isolation. In some embodiments, cell types in co-culture mimic the structural, biochemical, and/or phenomenological association observed between the cell types in vivo.

“Immunotherapy” refers to any medical intervention that induces, suppresses, or enhances the immune system of a patient for the treatment of a disease. In some embodiments, immunotherapies activate a patient's innate and/or adaptive immune responses (e.g., T cells) to more effectively target and remove a pathogen or cure a disease, such as cancer or an immune disease.

“Organoid” refers to a cellular structure obtained by expanding adult (post-embryonic) epithelial stem cells, consisting of tissue-specific cell types that self-organize through cell sorting and spatially restricted lineage commitment.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions, and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody or antibody fragment of the invention can be replaced with other amino acid residues from the same side chain family and the altered antibody or antibody fragment can be tested for the ability to bind CD123 using the functional assays described herein.

Unless otherwise specified, a “nucleotide sequence encoding an amino acid sequence” includes all nucleotide sequences that are degenerate versions of each other and encode the same amino acid sequence. Nucleotide sequences that encode proteins or RNA may also include introns to the extent that the nucleotide sequence encoding the protein may contain an intron(s) in some version.

“Effective amount” or “therapeutically effective amount” are used interchangeably herein and refer to an amount of a compound, formulation, material, or composition, as described herein effective to achieve a particular biological result. Such results may include but are not limited to the inhibition of virus infection as determined by any means suitable in the art.

As used herein, “endogenous” refers to any material from or produced inside an organism, cell, tissue, or system.

As used herein, the term “exogenous” refers to any material introduced from or produced outside an organism, cell, tissue, or system.

The term “expression” as used herein is defined as the transcription and/or translation of a particular nucleotide sequence driven by its regulatory sequences.

A “transfer vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate the transfer of nucleic acid into cells, such as, for example, polylysine compounds, liposomes, and the like. Examples of viral transfer vectors include, but are not limited to, adenoviral vectors, adeno-associated virus vectors, retroviral vectors, lentiviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

A “lentiviral vector” is a vector derived from at least a portion of a lentivirus genome, including especially a self-inactivating lentiviral vector as provided in Milone et al., Mol. Ther. 17(8): 1453-1464 (2009). Other Examples or lentivirus vectors that may be used in the clinic as an alternative to the pELPS vector, include but not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “operably linked” or alternatively “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

“Parenteral” administration of an immunogenic composition includes, e.g., subcutaneous (s.c.), intravenous (i.v.), intramuscular (i.m.), or intrasternal injection, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals including human).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

By the term “synthetic” as it refers to a nucleic acid or polypeptide, including an antibody, is meant a nucleic acid, polypeptide, including an antibody, which has been generated by a mechanism not found naturally within a cell. In some instances, the term “synthetic” may include and therefore overlap with the term “recombinant” and in other instances, the term “synthetic” means that the nucleic acid, polypeptide, including an antibody, has been generated by purely chemical or other means.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses.

By the term “specifically binds,” as used herein, is meant an antibody or antigen binding fragment thereof, or a ligand, which recognizes and binds with a cognate binding partner present in a sample, but which antibody, antigen binding fragment thereof or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Organoids

In one embodiment, the invention relates to ex vivo culture platforms and methods of using said ex vivo culture systems, wherein the ex vivo culture platform comprises organoids and/or organoid co-cultures obtained from epithelial cells of a subject. The ex vivo culture platforms allow for the investigation of subject-specific responses of organoids and/or organoid co-cultures to various test agents or test conditions.

In some embodiments, an organoid is a three-dimensional cellular structure. In some embodiments, an organoid is grown as a monolayer. In some embodiments, an organoid comprises a lumen surrounded by epithelial cells. In some embodiments, the epithelial cells surrounding the lumen are polarized. In some embodiments, the epithelial cells from which organoids are obtained are primary epithelial cells.

Organoids and/or organoid co-cultures may be obtained from normal (i.e., non-disease) epithelial cells or disease epithelial cells (sometimes specifically referred to as ‘disease organoids’ or ‘disease co-cultures’). Any epithelial cell from which an organoid can be generated is suitable for use in the invention. In one embodiment, the epithelial cells are neuroepithelial, including, but not limited to, cortical epithelial cells.

In one embodiment, the organoids are obtained from a subject from a specific population, such as a population characterized by gender, weight, body-mass index, disease state, ethnicity, age, responsiveness to treatment, or genetics. For example, in certain embodiments, the subject from which the organoid is obtained has a specific genotype. However, the present ex vivo culture platform, and uses thereof, is not limited to any particular subject but can be used to investigate subject-specific responses for any subject of interest.

In one embodiment, provided is a method for preparing a microglial cell comprising cortical organoid culture or co-culture. In some embodiments, the method comprises culturing cortical epithelial cells in contact with an extracellular matrix in an organoid culture medium comprising one or more additional agents to obtain an organoid culture or co-culture. In some embodiments, the method comprises culturing cortical epithelial cells in contact with an extracellular matrix in an organoid culture medium, removing said extracellular matrix and organoid culture medium from said organoid, and resuspending said organoid in a cell culture medium supplemented with one or more additional agent.

The extracellular matrix used to prepare microglial cell comprising cortical organoids, according to the invention, may be a hydrogel, foam or non-woven fiber. In some embodiments, the matrix is a hydrogel.

In one embodiment, the microglial cell comprising cortical organoids are prepared by co-culturing human epithelial stem cells containing an inducible Pu.1 transcription factor gene (Pu.1ⁱ-hESCs) with human epithelial stem cells without the inducible Pu.1 transcription factor gene (hESCs) in neural induction media for at least 8, 9, 10 11, 12, 13, 14, 15, 16, 17, or 18 days. In some embodiments, the neural induction media comprises an inducer molecule for induction of expression of the Pu.1 transcription factor.

In some embodiments, the ratio of Pu.1ⁱ-hESC:hESCs is 1:99 to 99:1 or any ratio therebetween. In some embodiments, the ratio of Pu.1ⁱ-hESC:hESCs is about 1:25, about 1:20, about 1:15, about 1:10, about 1:5 or about 1:1. In one embodiment, the ratio of Pu.1ⁱ-hESC:hESCs is about 1:9, about 1:3 or about 1:1.

In some embodiments, the neural induction medium comprises Dulbecco's Modified Eagle Medium/Nutrient Mixture F-12 (DMEM-F12) with KnockOut Serum Replacement (KSR), heat-inactivated FBS, Glutamax, minimum essential medium non-essential amino acids (MEM-NEAA), and β-Mercaptoethanol and supplemented with one or more of an inhibitor of the TGF-beta, Activin and Nodal signaling pathway, a TGF-Smad inhibitor, a Tankyrase inhibitor, and an inhibitor of Rho-associated, coiled-coil containing protein kinase (ROCK). In some embodiments, the neural induction medium comprises DMEM-F12 with 15% (v/v) KSR, 5% (v/v) heat-inactivated FBS, 1% (v/v) Glutamax, 1% (v/v) MEM-NEAA, 100 μM β-Mercaptoethanol; supplemented with 10 μM SB-431542, 100 nM LDN-193189, 2 μM XAV-939 and 50 μM Y27632.

In one embodiment, basal activation of the Pu.1 transcription factor is implemented by adding the inducer molecule to the cells in the neural induction medium on at least day 1, 2, 3, 4, 5, 6, 7, 8, 9, 10 or after day 10. In one embodiment, basal activation of the Pu.1 transcription factor is implemented by adding the inducer molecule to the cells in the neural induction medium on days 2, 3, 4, 5, 6, 7, 8, 9 and 10. In one embodiment, the Pu.1 is inducible by doxycycline, added to the neural induction medium beginning on day 2. In one embodiment, basal activation of the Pu.1 transcription factor is implemented by adding 0.5 μM dox beginning on day 2.

In one embodiment, FBS is removed from the neural induction medium beginning on day 2. In one embodiment, Y27632 is removed from the neural induction medium beginning on day 4.

In some embodiments, following at least 8, 9, 10, 11, 12, 13, 14, 15, 16, 17 or 18 days in the neural induction medium the cells are transferred to a spinning human cortical organoid culture medium lacking vitamin A, comprising a 1:1 mixture of DMEM-F12 and Neurobasal media, 0.5% (v/v) N2 supplement, 1% (v/v) B27 supplement without vitamin A, 0.5% (v/v) MEM-NEAA, 1% (v/v) Glutamax, 50 μM (3-Mercaptoethanol, 1% (v/v) Penicillin/Streptomycin and 0.025% Insulin. In one embodiment, the cells are transferred from the neural induction medium to the spinning human cortical organoid culture medium lacking vitamin A following 10 days of culture in the neural induction medium.

In some embodiments, following at least 4, 5, 6, 7, 8, or more than 8 days in the spinning human cortical organoid culture medium lacking vitamin A the cells are transferred to a human cortical organoid culture medium with vitamin A, comprising a 1:1 mixture of DMEM-F12 and Neurobasal media, 0.5% (v/v) N2 supplement, 1% (v/v) B27 supplement, 0.5% (v/v) MEM-NEAA, 1% (v/v) Glutamax, 50 μM β-Mercaptoethanol, 1% (v/v) Penicillin/Streptomycin and 0.025% Insulin. In one embodiment, the cells are transferred from the spinning human cortical organoid culture medium lacking vitamin A to the human cortical organoid culture medium with vitamin A following 8 days of culture in the neural induction medium.

In one embodiment, full induction of the Pu.1 transcription factor is implemented by adding the inducer molecule to the cells in the human cortical organoid culture medium with vitamin A. In one embodiment, full induction of the Pu.1 transcription factor is induced by doxycycline, added to the human cortical organoid culture medium with vitamin A. In one embodiment, full induction of the Pu.1 transcription factor is induced by adding about 2 μM dox continuously in the media.

### Agents

In some embodiments, the microglial containing human cortical organoids of the invention can be used to identify novel therapeutic agents or to evaluate the efficacy of known therapeutic agents. Exemplary agents that can be evaluated using the microglial containing human cortical organoids include, but are not limited to, peptidic agents, nucleic acid agents, small molecule agents.

Agents to be evaluated using the microglial containing human cortical organoids of the invention may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In certain instances, the system of the invention can be used to identify small molecules that have modulated properties such as potency, selectivity, and solubility, or that may serve as useful leads for drug discovery and drug development.

In some instances, a small molecule agent may be a derivative or analog of a known therapeutic agent. Analogs or derivatives of small molecules can be prepared by adding and/or substituting functional groups at various locations. As such, small molecules can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of a lead small molecule or can be based on a scaffold of a lead small molecule, but differing from it in respect to certain components or structural makeup, which may have a similar or opposite action metabolically.

In one embodiment, the small molecule agents can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Peptide Agents

In one embodiment, the agent is a protein or antibody agent. As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, NY). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, NY).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, NY) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide.

### Nucleic Acid Agents

In some embodiments, the agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule.

In one embodiment, the agent is an siRNA molecule. siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In certain embodiments, the nucleic acid agents are short hairpin RNA (shRNA) agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

A polynucleotide agent may be modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In one embodiment of the invention, the agent is an antisense molecule. Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules may be made synthetically and then provided to the organoid. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment the agent is a ribozyme. Ribozymes are useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In one embodiment, the agent may comprise one or more components of a CRISPR-Cas9 system, where a guide RNA (gRNA) targeted to a gene encoding a target molecule, and a CRISPR-associated (Cas) peptide form a complex to induce mutations within the targeted gene. In one embodiment, the agent comprises a gRNA or a nucleic acid molecule encoding a gRNA. In one embodiment, the agent comprises a Cas peptide or a nucleic acid molecule encoding a Cas peptide.

In one embodiment, the agent comprises a miRNA or a mimic of a miRNA. In one embodiment, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration.

In certain embodiments, the agent comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In one embodiment, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide agent has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances, include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

### Methods of Evaluating Responsiveness or Susceptibility

In one embodiment, the invention provides methods of evaluating the responsiveness of microglial containing human cortical organoid culture or co-culture to an agent of interest. For example, in one embodiment, the method comprises contacting microglial containing human cortical organoid culture or co-culture with an agent of interest and detecting one or more change in the organoid culture or co-culture indicative of a response to the agent. For example, in some embodiments, the detected change can be based on identification of an increase or decrease in cell viability, organoid size (e.g., surface area), morphology (e.g., budding), an alteration in protein levels or post-translational modifications of proteins, metabolism, production of soluble factors, an alteration in the quantification of epithelial subsets, cell proliferation, or any combination thereof, of the microglial containing human cortical organoid cells as compared to a comparator control.

In one embodiment, the method comprises evaluating the susceptibility of the microglial containing human cortical organoid culture or co-culture to injury mediated by an agent. For example, in one embodiment, the method comprises contacting the microglial containing human cortical organoid culture or co-culture with an agent of interest and detecting a change in cell health or cell viability. For example, in some embodiments, the detected change can be based on identification of an increase or decrease in cell viability, organoid size (e.g., surface area), morphology (e.g., budding), an alteration in protein levels or post-translational modifications of proteins, metabolism, production of soluble factors, an alteration in the quantification of epithelial subsets, cell proliferation, or any combination thereof, of the microglial containing human cortical organoid cells as compared to a comparator control. In one embodiment, the method is used to predict whether a subject diagnosed with a neurodevelopmental or neurodegenerative disease or disorder would be responsive to a treatment or therapeutic agent of interest. In one embodiment, the subject has or is suspected of having autism, schizophrenia, Alzheimer's disease (AD) or another dementia, Parkinson's disease (PD) or a PD-related disorder, frontotemporal dementia (FTD), amyotrophic lateral sclerosis (ALS), Huntington's disease (HD), spinocerebellar ataxias (SCAs), prion disease, spinal muscular atrophy (SMA), lewy body dementia (LBD), multisystem atrophy, primary progressive aphasia, multiple sclerosis (MS), ischemic stroke, traumatic brain injury, HIV-associated dementia, or another neurodegenerative, neurological or psychiatric disease or disorder.

### Methods of Testing Therapeutic Agents

In one embodiment, the invention provides methods of analyzing the effect of a test compound on a specific tissue microenvironment (e.g., a human neural microenvironment). In some embodiments, the invention provides methods for identifying a therapeutic agent for the treatment of a neurodevelopmental or neurodegenerative disease or disorder, comprising contacting a microglial containing human cortical organoid culture or co-culture with one or more candidate agents and detecting the presence or absence of one or more change in the microglial containing human cortical organoid culture or co-culture that is indicative of therapeutic efficacy. In some embodiments, a candidate agent is identified as a therapeutic agent based on identification of an increase in cell viability, organoid size (e.g., surface area), morphology (e.g., budding), an alteration in protein levels or post-translational modifications of proteins, metabolism, production of soluble factors, an alteration in the quantification of epithelial subsets, cell proliferation, or any combination thereof, of the microglial containing human cortical organoid cells as compared to a comparator control. In some embodiments, a candidate agent is identified as a therapeutic agent if there is at least one change in the transcriptome, proteome or secretome of the microglial containing human cortical organoid cells as compared to a comparator control.

### Kits

The invention also includes a kit comprising one or more of the compositions described herein. For example, in one embodiment, the kit comprises Pu.1ⁱ-hESCs and hESCs for use in generating a microglial containing human cortical organoid as described herein. In one embodiment, the kit comprises instructional material which describes the use of the composition. For instance, in some embodiments, the instructional material describes the steps for culturing the Pu.1ⁱ-hESCs and hESCs to generate a microglial containing human cortical organoid as described elsewhere herein. In some embodiments, this kit further comprises an inducible molecule and media or media supplements for use in the methods of generating a microglial containing human cortical organoid of the invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Human Cortical Organoids with Engineered Microglia-Like Cells

As brain resident myeloid cells, microglia play a critical role in the homeostasis of the brain. The dysregulation of microglia is an underlying mechanism in most human brain diseases, including AD, ASD, and Schizophrenia. However, there has been a significant challenge to develop a human in vitro model to investigate microglia, with the primary microglia (Galatro et al., 2017, Nat Neurosci, 20:1162-1171) or IPSCs-derived microglia 56-58 as viable options. Recent studies have revealed that microglial functions and the related phenotypes cannot be studied without considering the interaction with other cells in the brain (Gosselin et al., 2017, Science, 356; Haenseler et al., 2017, Stem Cell Reports, 8:1727-1742). Investigating the molecular and cellular responses in microglia necessitates the production of microglia-like cells in the context of a brain structure. Thus, the novel strategy developing mhCOs opens an opportunity to examine human microglia function in health and disease state closer to brain microglia.

Human brain organoid technology holds great potential to serve as a platform to model diseases and screen drugs for human brain diseases. However, most brain organoids made thus far lack microglia except for the recently reported cerebral organoids, unguided protocol (Ormel et al., 2018, Nat Commun, 9:4167). Here, PU.1-induction was employed during hCO formation, guided protocol, to differentiate hPSCs towards functional microglia-like cells, generating mhCOs. However, cerebral organoids still acquire mesodermal progenitors since they rely on the intrinsic differentiation of hESCs (Quadrato et al., 2017, Nature, 545:48-53). Therefore, Ormel and his colleagues modified the unguided protocol by decreasing the neuroectoderm stimulant and delaying Matrigel coating. Hence, these modifications lead to the production of mesodermal progenitors and following differentiation of microglia in organoids (Ormel et al., 2018, Nat Commun, 9:4167). While they noted innately developed microglia within cerebral organoids, variability of microglia numbers could become an issue. In contrast, the mhCOs described herein offer a platform for developing a tunable number of microglia within organoids and studying the microglia-specific function in vitro by genetic modification at either microglia alone or all cells in hCOs. This feature was utilized to determine the role of AD-associated genes in microglia in responding to Aβ by applying pooled CRISPRi screening. Furthermore, incorporating microglia-like cells in mhCOs does not require the steps to differentiate microglia from hESCs and co-culture with hCOs, and mhCOs carry microglia-like cells parenchyma as well as the surface of organoids, closely producing the location and behavior of microglia in the human brain. In addition, generating different types of organoids with PU.1 expressing cells may lead to the production of myeloid cells with a distinct identity and maturation efficiency. Lastly, mhCOs open up new avenues to probe the function of microglia readily in human brain organoids for a variety of human brain disorders as well as brain development.

The materials and methods used in the experiments are now described

### Animals

The Rag2−/− GammaC−/− mice were purchased from Jackson Laboratories.

hESCs Culture

HES-3 NKX2-1^{GFP/w}, BC4 and BC61 hESCs were cultured on Matrigel (BD Biosciences) coated cell culture dishes with mTeSR1 media (Stem Cell Technologies). hESCs were passaged every week by treatment with Dispase (0.83 U/ml, Stem Cell Technologies).

Generation of BC61 hESCs

A cassette containing doxycycline-inducible PU.1-IRES-eGFP and rTTA was generated and introduced into the AAVS1 locus of HES3. Briefly, 2 million HES3 cells were electroporated with 8 μg donor plasmid, 1 μg AAVS1 TALEN-L, and 1 μg AAVS1 TALEN-R by using the Amaxa Nucleofector device (AAB-1001, Lonza) and seeded in mTeSR1 plus Y27632 (10 μM). After 3 days, G-418 (Thermo Fisher Scientific) was applied for 7 days (400 μg ml⁻¹ for the first 3 days and 300 μg ml⁻¹ for the next 4 days) to obtain stable colonies. A single isogenic colony was picked and expanded for quality control analysis.

Generation of Human Cortical Organoids (hCOs) with PU.1 Induction

To generate microglia containing cortical organoids, lentivirus containing PU.1 was generated. Firstly, PU.1 gene is amplified from pINDUCER-21-SPI1 (addgene #97039) using primers forward: 5′-TTTGGATCCAAGGCCCACCATGGAAGGGT-3′ (SEQ ID NO:73) and reverse: 5′-TTTGTTTAAACTCATTACTAAGCGTAGTCTG-3′ (SEQ ID NO:74) and cloned into pTet-IRES-eGFP (addgene #64238) with PmeI and BamHI. The lentivirus containing inducible Pu.1 was transduced for 7 days in the absence of dox. hCOs were generated by mixing 10% PU.1-infected BC4 and 90% non-infected parental HES3 hESCs. Briefly, 9000 cells, 900 PU.1-infected and 8100 cells HES3, were plated into a well of U-bottom ultra-low-attachment 96-well plate in neural induction media (DMEM-F12, 15% (v/v) KSR, 5% (v/v) heat-inactivated FBS (Life Technologies), 1% (v/v) Glutamax, 1% (v/v) MEM-NEAA, 100 μM β-Mercaptoethanol) supplemented with 10 μM SB-431542, 100 nM LDN-193189, 2 μM XAV-939 and 50 μM Y27632. Basal activation of PU.1 was started on day 2 by adding 0.5 μM dox, and FBS and Y27632 were removed from day 2 and 4, respectively. The media was replenished every other day until day 10, where organoids were transferred to an ultra-low-attachment 6-well plate. The organoids were cultured in spinning hCO media with minus vitamin A (1:1 mixture of DMEM-F12 and Neurobasal media, 0.5% (v/v) N2 supplement, 1% (v/v) B27 supplement without vitamin A, 0.5% (v/v) MEM-NEAA, 1% (v/v) Glutamax, 50 μM β-Mercaptoethanol, 1% (v/v) Penicillin/Streptomycin and 0.025% Insulin). The media was replenished every other day until day 18, where media was switched to the hCO media with vitamin A (the same composition as described above except B27 with vitamin A) supplemented with 20 ng/ml BDNF and 200 μM ascorbic acid. The media was changed every 4 days after day 18. Activation of PU.1 was performed beginning on day 18 by adding 2 μM dox continuously in the media.

### Human Tissue

Human tissue was obtained and immediately placed in an RPMI medium and processed within 10 hours of collection. Briefly, 70- and 77-year old brain tissues were cut into 1-mm sections and cultured in hCO culture media in the presence and absence of Aβ₁₋₄₂-oligo for 72 hours. Samples were collected and processed for analyses.

Live Imaging of mhCOs Types

Live images were captured in mhCOs at day 30 and day 70 and mhCO at day 60 to visualize the eGFP-expressing cells and their morphologies. The organoids bearing GFP+ cells, imaged for x, y, z, t scanning mode obtaining z stack images for 1-4 hours with 10 minute time intervals. The Leica TCS SP5 confocal microscope, equipped with a controlled cell chamber possessing 37° C. temperature and 5% C02, was used to generate z stack images. 4D reconstruction of images was attained by using Leica Las-X software.

### Scanning Electron Microscopy

The cultured organoids day 75 were fixed in 2.5% glutaraldehyde solution for 30 min. Then, the organoids were applied to a sequential dehydration series of 50%, 75%, 90% and 100% ethanol. Finally, the surfaces of the hCOs were sputter-coated with gold and imaged by FE-SEM (Model S-4700, Hitachi, Canada).

### Immunofluorescence Staining

For organoids, residual media was removed by washing with PBS. All hCOs were fixed in 4% paraformaldehyde (PFA) at 4° C. overnight. After washing with PBS three times, they were incubated in 30% sucrose solution for 2 days at 4° C. Organoids were embedded in O.C.T in base molds on dry ice and sectioned for 40-μm. The organoid blocks were further stored at −80° C. After sections were dried, they were incubated with 0.1% Triton-100 for 15 min and further blocked with 3% bovine serum albumin (BSA) for 2 hours at RT. Then, the primary antibody, diluted in 3% BSA, incubation is performed at 4° C. overnight. After washing with PBS, organoids were incubated with Alexa Fluor dyes (1:1000) for 1 hour and following nuclei staining with DAPI (1:1000) for 10 minutes at RT. Finally, slides were mounted with ProLong Gold Antifade Reagent and images were taken with Leica TCS SP5 confocal microscope. The tunnel assay (C100247, Invitrogen) was performed to detect apoptotic cells following the manufacturer's protocol. A list of antibodies is presented in Table 1.

TABLE 1
List of antibodies used for Immunostaining.

| Antibody | Source | Identifier |
|---|---|---|
| IBA1 | Wako | Cat# 019-19741 |
| CD68 | Abcam | Cat# ab31630 |
| CD68 | Abcam | Cat# ab199000 |
| C1qC | R&D Systems | Cat# AF3337 |
| TMEM119 | Atlas Antibodies | Cat# AMAb91528 |
| PU.1 | ThermoFisher | Cat# Ma5-15064 |
| CSF1R | Thermo Fisher | Cat# Pa5-25974 |
| LAMP1 | Cell Signaling | Cat# 15665S |
| P2RY12 (4H5L19) | ThermoFisher | Cat# 702516 |
| β-Amyloid (D54D2) | Cell Signaling | Cat# 8243S |
| Ki67 | BD Biosciences | Cat# 556003 |
| Caspase-3 | Cell Signaling | Cat# 9661S |
| NRXN1 | Novus Biologicals | Cat# NBP2-68920 |
| GFP | Sigma | Cat# SAB4600051 |
| MAP2 | Millipore | Cat# MAB3418 |
| PSD95 | Abcam | Cat# ab12093 |
| TBR2 | Abcam | Cat# ab23345 |
| SATB2 | Abcam | Cat# ab51502 |
| CTIP2 (25B6) | Abcam | Cat# ab18465 |
| SOX2 | Cell Signaling | Cat# 3579 |

### Whole-Mount Immunostaining of Organoids

Whole-mount immunostaining was performed followed by confocal microscopy to examine the localization and organization of Aβ deposition and microglia localization within the cortical organoids. Whole-mount immunostaining of organoids was applied. Briefly, organoids were fixed overnight in 4% paraformaldehyde (PFA) at 4° C. After extensive washing the organoids with PBS around 3 to 5 hours, the organoids were blocked overnight at RT in 0.5% BSA and 0.125% Triton-100 in PBS. Organoids were incubated in primary antibodies (anti-IBA1 1:100, anti-MAP2 1:200, anti-PSD95 1:200, anti-CD14 1:100, anti-Aβ 1:100 and anti-C1qC 1:200) and diluted in 0.5% BSA and 0.125% Triton-100 in PBS for 2-days at 4° C. Unbound antibodies were removed via washing with PBS for one day at RT. Then, organoids were incubated with Alexa Fluor Dyes (1:500) for 4 h following nuclei staining with DAPI (1:1000) for 2 hours. The organoids were cleared by applying to a sequential dehydration series of 30%, 50%, 70% and 99% 1-Propanol (diluted in PBS pH adjusted to 9.5 via triethylamine) for 4 hours at 4° C. After dehydration, the organoids were incubated with ethyl cinnamate for 1 h at RT in light protected and air-sealed tubes. The images were taken with a Leica TCS SP5 confocal microscope.

### Single-Molecule RNA Fluorescent In Situ Hybridization

For sample preparation, whole organoids at day 90 were frozen in Tissue-Tek Cryo-OCT compound (Fisher 811 Scientific) on dry ice and stored in −80° C. until further use. Organoids were sectioned at a thickness of 20 μm and RNAs were detected by RNAscope (Advanced Cell Diagnostics) based on the manufacturer's guidelines using the probes for human Fos (check the cat. no 319901) and Npas4 (check the cat. no 501121-c2). All processed samples were imaged using an Olympus VS120 slide scanner and analyzed in ImageJ.

### Viral Labeling and Calcium Imaging

Organoids were transferred to a 96-well plate for viral infection. After AAV. Syn. GCAMP6s.WPRE.SV40 (Addgene, 100843, (Chen et al., 2013, Nature, 499:295-300) and AAV5.hSyn. eGFP (Addgene, 50465) separate incubation in 300 μl neural media for 24 hours, organoids were transferred to 6-well plate in fresh medium. After 10 to 15 days of virus transduction, the intact organoids were used for calcium and structural imaging. Time-lapse images were taken with Leica TCS SP8 confocal microscope at a speed of 1s/frame. Tracings of single-cell calcium surges were determined by measuring the region of interest and mean of interest fluorescence intensities using Fiji software (Schindelin et al., 2012, Nat Methods, 9:676-682). The change in calcium concentration is calculated as follows ΔF/F=[(F(t)−F0)/F0), where F0 is calculated as the average of portions without calcium events.

Real-Time Quantitative PCR (qPCR)

Total RNA was isolated from the whole organoids via RNeasy Mini Kit (Qiagen). 1 μg RNA was converted to cDNA using iScript Select cDNA Synthesis Kit. For the quantification of gene expression, qPCR was carried out on the CFX96 Real-Time PCR system (Biorad) using the SsoFast EvaGreen Supermix (Biorad). The PCR conditions were: 95° C. for 15 minutes, followed by 40 two-step cycles at 94° C. for 10 seconds and 60° C. for 45 seconds. A list of primers used in this study is presented in Table 2.

TABLE 2
List of primers used for qPCR experiment

| | SEQ | | SEQ | |
|---|---|---|---|---|
| | ID | | ID | |
| Gene | NO: | Forward Primer | NO: | Reverse primer |
| PU.1 | 1 | CGACCATTACTGGGACTTCCA | 2 | GGAGCTCCGTGAAGTTGTTCTC |
| IBA1 | 3 | GTCCCTGAAACGAATGCTG | 4 | CCTTTTCTCTCGCTTTTTCCTC |
| LAG3 | 5 | CGTCTCCATCATGTATAACCTC | 6 | GTAAAGTCGCCATTGTCTCC |
| CX3CR1 | 7 | CAACAGCAAGAAGCCCAAG | 8 | CGATGAAGAAGAAGGCGGTAG |
| CD11b | 9 | TCCAGAACAACCCTAACCC | 10 | CGCCAAACTTTTCTCCATCC |
| TMEM119 | 11 | GCCTCCTCATCCTTCTGTTG | 12 | AAGAAGTCCACTATCCCATCC |
| P2RY12 | 13 | ACATCCAACCCCAAAAATCTC | 14 | TTTACCTACACCCCTCGTTC |
| SALL1 | 15 | TCACAACCTCTCTACCTCAAC | 16 | TGCATTCTGAGAAGCCAAC |
| GPR34 | 17 | CCAACCGCCACAAAACTTC | 18 | TATGTTCCCAACCAGTCCC |
| AXL | 19 | CCATCCTCACACCCCTTATC | 20 | TCTTGCCTTAGCCCTATGTC |
| CSF1R | 21 | CACCAAGCTCGCAATCCCTC | 22 | CTCTACCACCCGGAAGAACA |
| MPO | 23 | ACCATCCGCAACCAGATCAA | 24 | GCTCCTCGCTGCCGTACA |
| IL3RA | 25 | CCGCATCCCTCACATGAAA | 26 | TCCCAGACCACCAGCTTGTC |
| CD33 | 27 | CACTTTCTTCCATCCCATACC | 28 | TGAACCATTATCCCTCCTCC |
| SORL1 | 29 | AAACTGCCCTACCACCATC | 30 | ACTTTCATCACTGTTGTCTCC |
| TREM2 | 31 | CACCCACTTCCATCCTTCTCC | 32 | TCCAGTTCACTGGGTGGATG |
| NRXN1 | 33 | CACCACATCCACCATTTCC | 34 | AAGAACTGTCCACTCGCAC |
| GPM6A | 35 | AAGAGGGACAGACACAAAAAG | 36 | CAGCAAAATGCCATACACAAAG |
| ANK3 | 37 | ACACACCACCTACCCCTTTC | 38 | TCTCATCTACATCCACTGCTTC |
| PAK3 | 39 | ATGGATGTGGATAGGCGAG | 40 | TGCAGCGATAATCAGAGGAG |
| PCLO | 41 | AAACAACCCCTCCTCTAAAAAC | 42 | TTTGACCTTTGCTCTCTGAAC |
| MAP6 | 43 | CGAGTCCGTGAAGAATCAAG | 44 | GGGTAGAGGTGAGACAGTAG |
| MAP2 | 45 | GCACGACTAAAAGGGCTAC | 46 | AGGAGGGAGAATGGAGGAAG |
| PTPRD | 47 | TTCTCTATCCCACCCACTAATC | 48 | ATCTTCTGCCCCCAACATC |
| DNM1L | 49 | AATTCCATTATCCTCGCTGTC | 50 | ACCCTTCCCATCAATACATCC |
| NTN1 | 51 | CCATCACCAAGCAGAACGAG | 52 | ACACCGCGTAGAAGTACGAG |
| POU5F1 (OCT4) | 53 | CCTCACTTCACTGCACTGTA | 54 | CAGGTTTTCTTTCCCTAGCT |
| TUJ1 | 55 | GCCGCTACCTGACGGTGGC | 56 | GGGCGGGATGTCACACACGG |

Library Preparation for scRNA-Seq

Cortical organoids were randomly collected from 3 different culture dishes for each time point (Day 90: hCOs and mhCOs with and without Aβ treated total 32 organoids pooled; day 75: totally 8 mhCOs pooled together to sort GFP⁺ and GFP⁻ cells). Organoid dissociation, cDNA preparation, and sequencing were performed. Briefly, organoids were dissociated using the papain according to the manufacturer's instructions. After washing once with HBSS, organoids were dissected into small pieces in oxygenated papain solution (all solutions used were oxygenated with 95% 02:5% C02 for around 5 minutes). Dissected tissue was oxygenated for 5 minutes and incubated in a 37° C. water bath for 1 hour, with gentle shaking every 10 minutes. After gentle trituration, papain was inactivated in albumin-ovomucoid inhibitor, and single cells were suspended in 1% BSA/PBS supplemented with 10 μM Y27632. Then, cells were stained with propidium iodide (PI) for 15 minutes on ice, sorted out, and re-suspended at 128 cells/μl. cDNA libraries were generated with the Single Cell 3′ v3 Reagent Kits according to the manufacturer's instructions. After barcoded full-length cDNA from poly-adenylated mRNA generated, the libraries were then size-selected, and R2, P5, P7 sequences were added to each selected cDNA during end repair and adaptor ligation. After Illumina bridge amplification of the cDNA, each library was sequenced using the Illumina Novaseq S4 2×150 bp in Rapid Run Mode.

Data Processing of scRNA-Seq

scRNA-seq reads were aligned to hg19 human genome and counted with Ensembl genes by count function of CellRanger (v3.0.2) with default parameters. All libraries were merged with the normalization to the same sequencing depth by aggr function of CellRanger with default parameters. Before processing scRNA-seq analysis, low doublet frequency of the scRNA-seq libraries (0.82±0.28%) was confirmed by counting cells expressing both TBR1 and GFAP, which are usually exclusively expressed in cortical neurons and astrocytes, respectively (Xiang et al., 2017, Cell Stem Cell, 21:383-398 e387; Xiang et al., 2019, Cell Stem Cell, 24:487-497 e487).

Batch effect and intrinsic technical effect were minimized by Seurat (v3.0.2) (Stuart et al., 2019, Cell, 177:1888-1902). Briefly, raw UMI count was normalized to total UMI count in each library. Highly variable genes were then identified by variance stabilizing transformation with 0.3 loess span and automatic setting of clip.max value. Top 2,500 variable genes were used to identify cell pairs anchoring different scRNA-seq libraries using 20 dimensions of canonical correlation analysis. All scRNA-seq libraires used in this study were integrated into a shared space using the anchor cells. After scaling gene expression values across all integrated cells, dimensional reduction was performed using principal component analysis (PCA). For the visualization, single cells were further projected into two-dimensional UMAP space from 1^{st} and 30^{th} PCs. Graph-based clustering was then implemented with shared nearest neighbor method from 1^{st} and 20^{th} PCs and 0.8 resolution value. Differentially-expressed genes (DEGs) in each cluster was identified with more than 1.25-fold change and p<0.05 by two-sided unpaired T test. Gene Ontology analysis was performed to the DEGs by GOstats Bioconductor package (v2.46.0). False discovery rate was adjusted by p.adjust function in R with “method=“BH”” parameter.

Cell types were then assigned systematically with unique markers and Gene Ontology enrichment and validated by the reference transcriptome of human brain cell types (Darmanis et al., 2015 Proc Natl Acad Sci USA, 112:7285-7290). First, neuronal clusters were isolated with expression of neuronal growth cone markers (STMN, GAP43 and DCX). Excitatory cortical neuron (CN) and interneuron clusters (IN) were then categorized by their unique markers (SLC17A6 for CN and SLC32A1 and GAD2 for IN). Neuronal clusters without these markers were assigned as non-committed neuron (Neuron).

Non-neuronal clusters were also separated with early neurogenesis markers (VIM, HES1 or SOX2). Clusters expressing or lacking both the growth cone and the early markers were assigned as intermediate (Inter). Neuronal progenitor cell clusters (NPCs) were categorized by high expression of genes related to “mitotic nuclear division (GO:0007067)”. As shown previously (Xiang et al., 2019, Cell Stem Cell, 24:487-497 e487) several non-neuronal clusters were characterized by “cilium assembly (GO:0042384)” and “response to BMP (GO:0071772)” and called as cilia-bearing cell (CBC) and BMP responsible cell (BRC), respectively. 10 non-neuronal clusters displayed high expression of “gliogenesis (GO:0042063)”. In particular, eight out of the glia clusters also highly expressed genes related to “astrocyte differentiation (GO:0048708)” and assigned as astrocyte clusters (AS). One cluster was annotated as unfolded protein response-related cell (UPRC) with significant enrichment of “endoplasmic reticulum unfolded protein response (GO:0030968)”.

Six non-neuronal clusters were predominantly generated from PU.1-induced cortical organoids. In particular, one of them was characterized by microglia-specific markers (SPI1 (PU.1), AIF1, CSF1R, C1QA and C1QB) and significant enrichment of “leukocyte mediated immunity (GO:0002443)”. Three clusters highly expressed biglycan (BGN) and decorin (DCN) and were annotated as proteoglycan-expressing cell (PGC). Two clusters were characterized by unique expression of claudin and keratin and assigned as claudin- (CLC) and keratin-enriched cell (KRC), respectively. One cluster was predominantly generated from Aβ-treated hCOs and characterized as mesodermal markers (MYL1 and MYH3). The rest cluster showed no significant enrichment of GO terms and was called an unassigned cluster (UN).

The reference transcriptome of fetal and adult brain was downloaded from NCBI Short Read Archive (SRP057196) (Darmanis et al., 2015 Proc Natl Acad Sci USA, 112:7285-7290). Gene signatures for each cell type were obtained as described previously (Xiang et al., 2017, Cell Stem Cell, 21:383-398 e387). In each cell, genes were ranked by relative expression to average of all cells. Gene Set Enrichment Analysis (GSEA) was conducted by GSEAPY software (v0.9.3) with options ““−max-size 50000-min-size 0-n 1000” to the pre-sorted genes.

In each library, 15 cells (approximately 0.15%) were set to define “generation” of each cell type. With this threshold count, non-treated hCOs did not generate MG (3 cell), CLC (4 cells), KRC (4 cells) and ME (0 cells). MG (12 cells) was also not generated from Aβ-treated hCOs. Both non-treated and Aβ-treated mhCOs did not generate ME (4 and 2 cells), respectively.

Global gene expression comparison between different organoids was performed by using all cells in scRNA-seq dataset. Differential expression was defined by p<1e-50 with two-sided T test. GO analysis was performed by GOstats as described above.

Single cell RNA-seq datasets of in vivo human brain from AD patients and healthy donors were downloaded from NCBI Gene Expression Omnibus (GEO) (GSE138852) (Grubman et al., 2019, Nat Neurosci, 22:2087-2097). The raw sequence reads were processed as described above and merged with the scRNA-seq datasets by CellRanger aggr function. The UMI count matrix was normalized and projected into the shared UMAP space with Seurat as described above. The annotation of the human brain samples was determined as that of the nearest cells from the organoid samples by calculating Euclidian distance from 1^{st} to 30^{th} PCs.

The UMI count matrix for droplet-based scRNA-seq of human fetal brain was downloaded from UCSC Cell Browser (cells.ucsc.edu/?ds=organoidreportcard) (Bhaduri et al., 2020, Nature, 578:142-148). The human fetal brain single cell transcriptome was integrated with the mhCO scRNA-seq by Seurat as described above. Single-cell transcriptome data from human fetal microglia was obtained from NCBI GEO (GSE141862) (Kracht et al., 2020, Science, 369:530-537), and integrated with cells in MG cluster from the scRNA-seq. Developmental trajectory was then constructed from the normalized expression values using Monocle (v2.99.3) (Cao et al., 2019, Nature, 566:496-502). Briefly, a principal tree was drawn by DDRTree algorithm and cells were grouped by Louvain clustering with 20 nearest neighbors. Developmental stage (Pseudotime) was then estimated by selecting a cluster that mainly composes of GW9 microglia. Pearson correlation coefficient between pseudotime and expression level was calculated by cor function in R for each gene. Gene Ontology analysis was implemented by GOstats for genes with more than 0.2 correlation coefficient.

scRNA-seq for GFP⁺ and GFP⁻ populations were preprocessed by cellranger (v.3.0.2) and Seurat (v3.0.2), as described above. After mapping and quality control, developmental trajectory was constructed from single cells from GFP+ and GFP− populations using DDRTree algorithm with 10 max components in Monocle (v2.99.3) (Cao et al., 2019, Nature, 566:496-502). Trajectory branches were assigned to cell types with their unique markers. To measure the efficiency of MG lineage commitment, cells with GFP and PU.1-derived reads were divided into 100-cells bin and the number of cells in MG branch in each bin were counted.

Gene expression profiles of microglia precursor (MP) and differentiated microglia (dMG) were obtained from NCBI GEO (GSE139194) (Svoboda et al., 2019, Proc Natl Acad Sci USA, 116:25293-25303). GSEA of gene signatures for MG, CEC and KEC clusters between dMG and MP was implemented by GSEA software (v2.2.2) with 1,000 permutations to gene set, no dataset collapse and weighted enrichment statistic (Subramanian et al., 2005, Proc Natl Acad Sci USA, 102:15545-15550).

Bulk RNA-seq datasets for AD patient brains in each Braak stage were downloaded from NCBI GEO (GSE110731) (Li et al., 2019, Nat Commun, 10:2246). The reads were mapped to hg19 human genome by Tophat2 (v2.2.1) with default parameters (Trapnell et al., 2009, Bioinformatics, 25:1105-1111). The gene expression value was estimated by Cufflinks (v1.2.0) with RefSeq gene reference annotation (Trapnell et al., 2010, Nat Biotechnol, 28:511-515). The stage-specific genes were identified with more than 1.25-fold change and p<0.05 by two-sided T test. The statistical enrichment of the stage-specific genes was evaluated by GSEA software (v2.2.2) as described above.

Ferroptosis-induced gene sets were obtained from gene expression profiles in withaferin A (WA)-treated cells (GSE112384) (Hassannia et al., 2018, J Clin Invest, 128:3341-3355). Up-regulated genes (log 2(fold change)>0.1 and p<0.05 by two-sided T test in both IMR32 and SK-N-SH cell lines) in WA-treated samples to control and with a non-treated sample were used as “ferroptosis genes”.

### Data Processing of CROP-Seq

CROP-seq reads were first aligned to gRNA plasmid library sequences by Bowtie2 (v2.3.0) (Langmead et al., 2012, Nat Methods, 9:357-359). For the annotation of cell types, CROP-seq datasets were merged with scRNA-seq datasets and processed by CellRanger and Seurat software as described above. The annotation of individual cell from CROP-seq dataset was labeled as that of the nearest cells from scRNA-seq by calculating Euclidean distance from first and 20^{th} PCs. Differentially-expressed genes between Aβ₁₋₄₂ and non-treatment mhCOs were identified in each knockdown with 1.25 fold change and p<0.05 by two-sided paired T test.

### Quantification of Cholesterol Levels

Cellular cholesterol levels were extracted from organoid tissue. Briefly, 10 mg tissues were dissolved with 200 μl of chloroform:isopropanol:NP40 (7:11:0.1) in a micro-homogenizer. After 10 minutes of room temperature incubation, tissue extract was spun at 15000 g for 10 minutes. The liquid was transferred to a glass tube and air-dried for 30 minutes to remove the organoid solution. The dried lipids were dissolved with 250 μl of cholesterol assay buffer by vortexing until homogenous. Then, the cholesterol levels were measured from 50 μl of the extracted sample using the Amplex Red cholesterol assay kit (Invitrogen), according to the manufacturer's instructions. Briefly, cholesterol oxidase converts cholesterol to hydrogen peroxide and ketones. The hydrogen peroxide reacts with Amplex Red reagent (10-acetyl-3,7-dihydroxyphenoxazine), producing resorufin, fluorescence monitored excitation/emission couple of 545/590 nm on a PerkinElmer plate reader.

Transplantation of PU.1 Induced Microglia into Mouse Brain

PU.1-derived microglia was transplanted into mice brain as previously described (Mancuso et al., 2019, Nat Neurosci, 22:2111-2116). Briefly, PU.1 induced for 5 days in BC4 hESCs to generate microglia-like cells. Then, these cells dissociated and suspended in PBS (100.000 cells/μl). 10 μM Aβ_oligo was prepared by dissolving Aβ in Tris-EDTA buffer (50 mM Tris and 1 mM EDTA, pH 7.5). After mice (postnatal day 4) were anestesized by hypothermia, 100K microglia-like cell suspension and 5 μl Aβ_oligo (10 μM) were bilaterally injected to mice brain. Then, mice were recovered on heating pad at 37° C. After 21-days of microglia transplantation, mice were perfused with PBS and 4% PFA. Then, explanted brain tissues were further fixed and sliced for further immunofluorescence staining.

### Statistics

Data are presented as mean±SEM. The paired or unpaired two-tail t-test (GraphPad Prism software version 8.2.0), hypergeometric test adjusted by Benjamini-Hochberg procedure, and two-sided t-test (R version 3.5.0 software) were used to determine the statistical significance. Statistical tests and biological replicates for each experiment are presented in the Figure legends.

The results of the experiments are now described.

### Induction of Microglia Via PU.1 Expression Under Different Conditions.

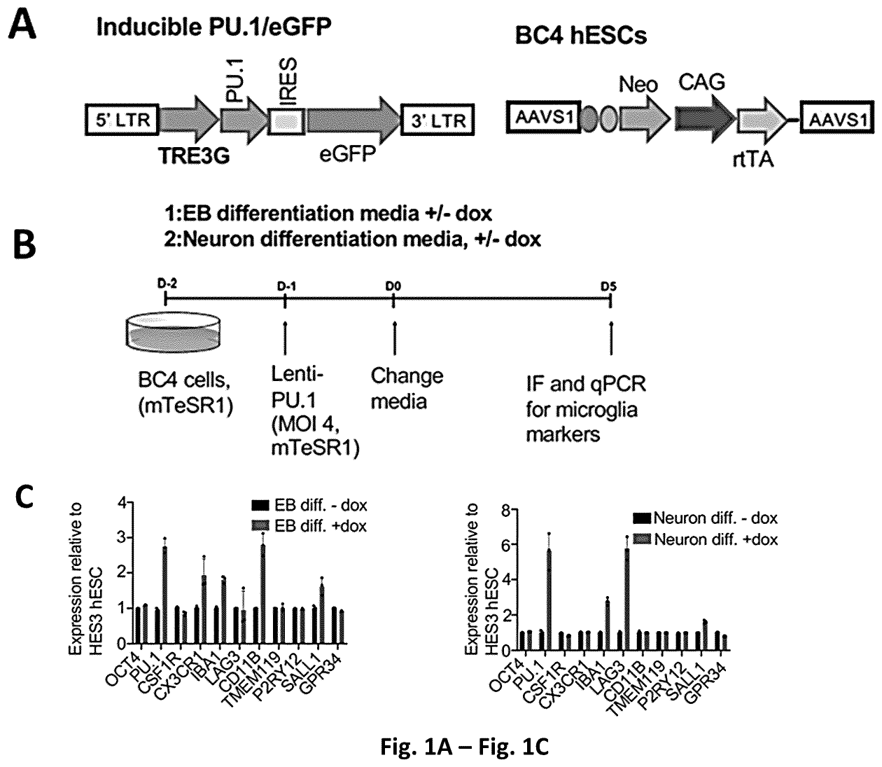

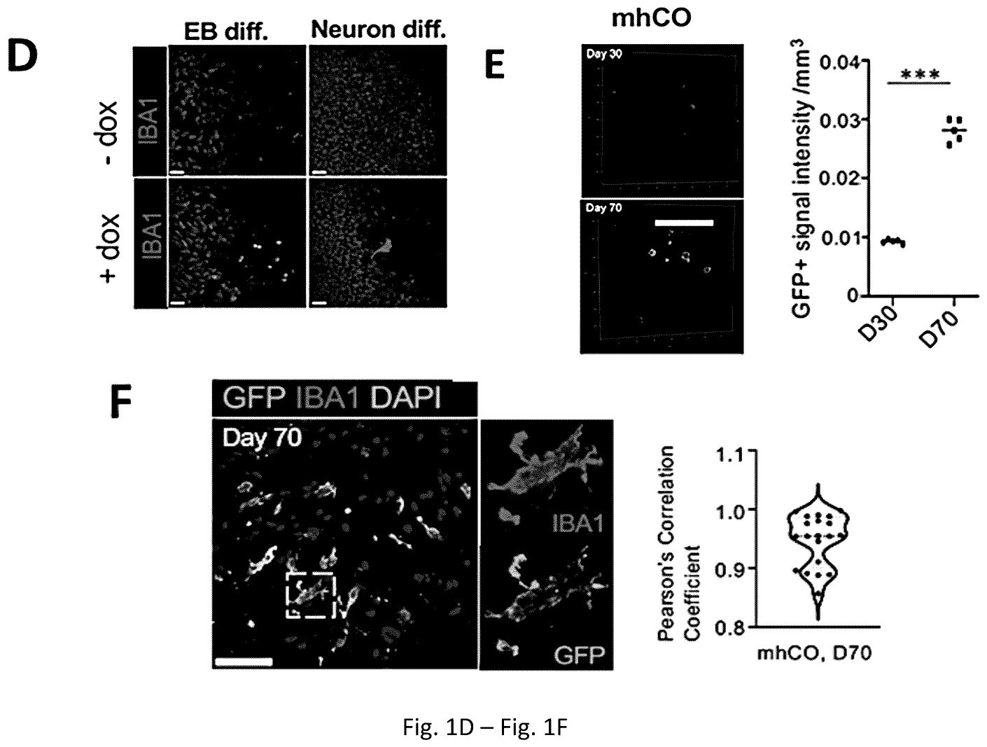

Previous studies demonstrated that an ETS-domain transcription factor called PU.1 (or SPI1) is critical in developing cells into the myeloid lineage, including microglia, and that overexpression of PU.1 reprograms the various cell types into myeloid cells (Kierdorf et al., 2013, Nat Neurosci, 16:273-280; Feng et al., 2008, Proc Natl Acad Sci USA, 105:6057-6062; Forsberg et al., 2010, Proc Natl Acad Sci USA, 107:14657-14661). Here, whether the PU.1-induced cells in hCOs could differentiate into functional microglia was examined. The BC4 hESC line expressing rTTA was utilized in a doxycycline-inducible manner to induce PU.1 using the lentivirus expressing eGFP. Two conditions were tested: embryoid body (EB) differentiation and neuron differentiation conditions (FIG. 1A and FIG. 1). In both settings, the 5-day PU.1-induction dramatically increased the expression of microglia markers such as IBA1 and limited induction of SALL1, as described previously (Galatro et al., 2017, Nat Neurosci, 20:1162-1171). In contrast, the expression of colony-stimulating factor 1 (CSF1R, CD115) regulating the microglia survival, differentiation, and proliferation, and other microglia markers such as TMEM119, P2RY12 were not increased (FIG. 1C). This limited induction of microglia markers was most likely caused by the short introduction of PU.1 and in vitro settings related to downregulation as described earlier (Gosselin et al., 2017, Science, 356). Immunostaining (IF) for IBA1 further revealed the presence of microglia-like cells in the differentiating cells that expressed PU.1 for 5-days under either EB or neuron differentiation conditions (FIG. 1D). Overall, these data demonstrate that PU.1 expression directs the differentiating hESCs into myeloid cells even in neuroectoderm differentiation conditions.

Engineering of hCOs with Functional Microglia (mhCO).

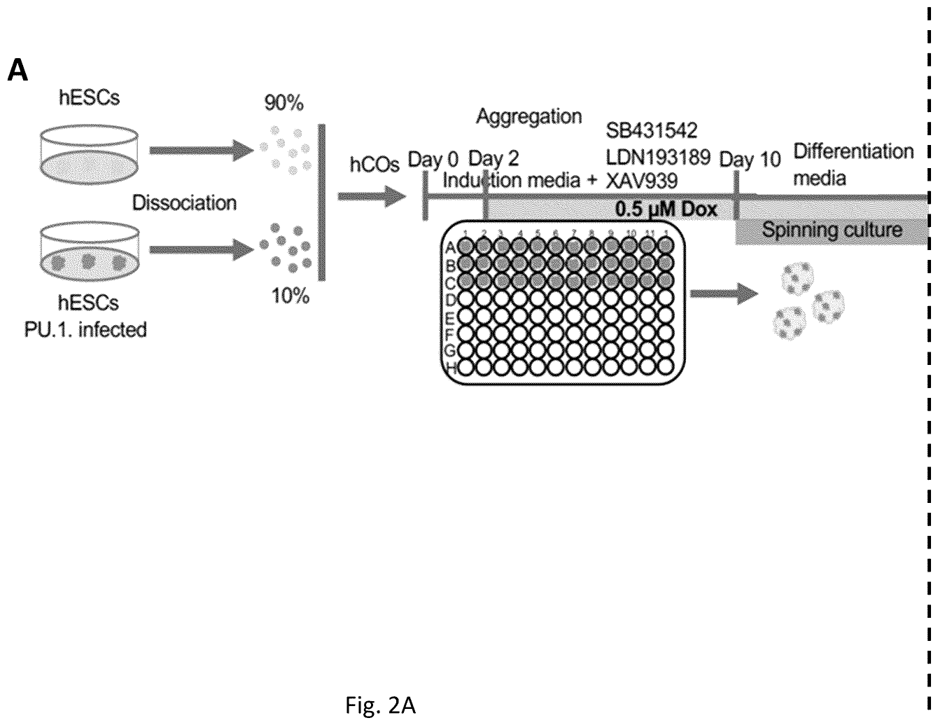

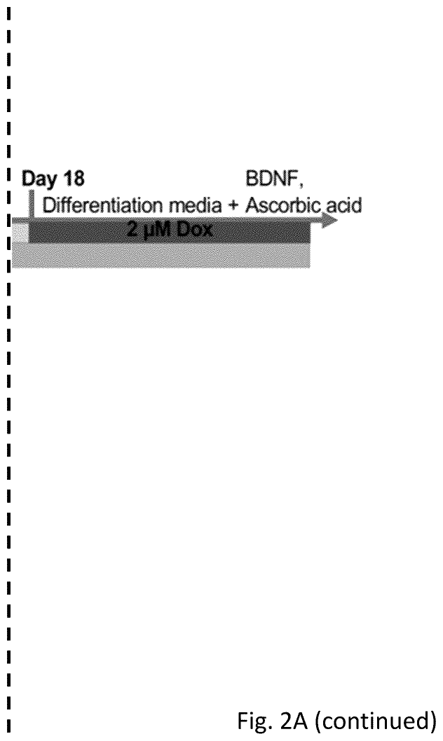

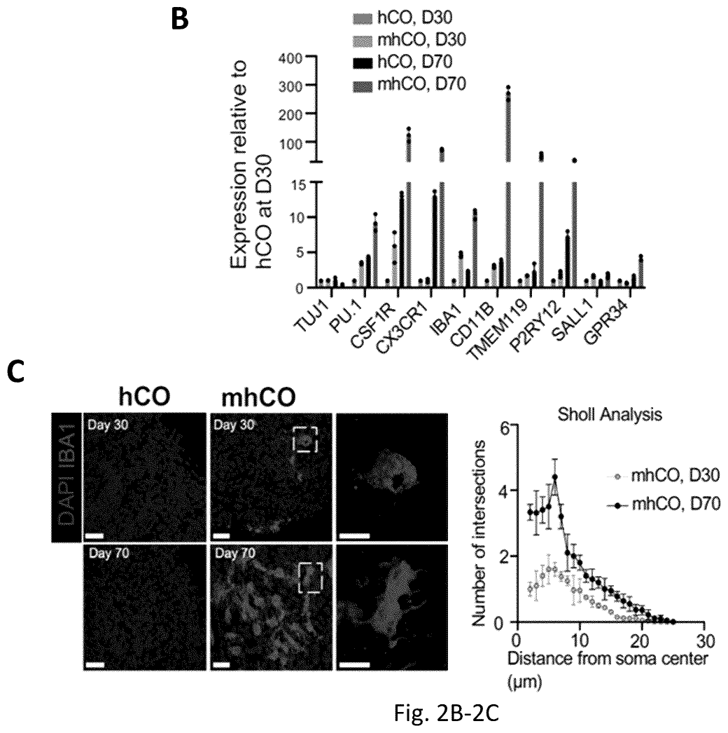

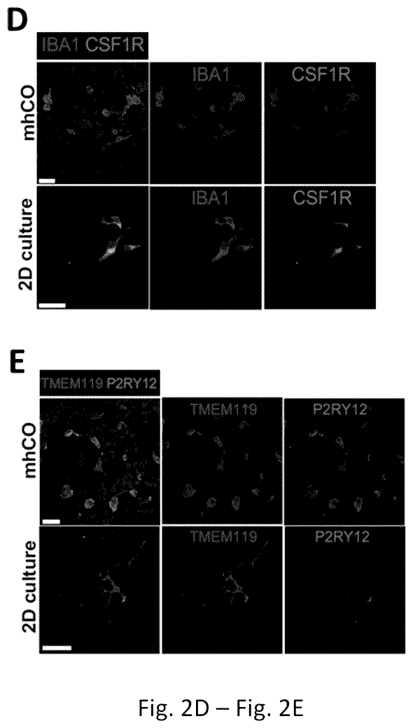

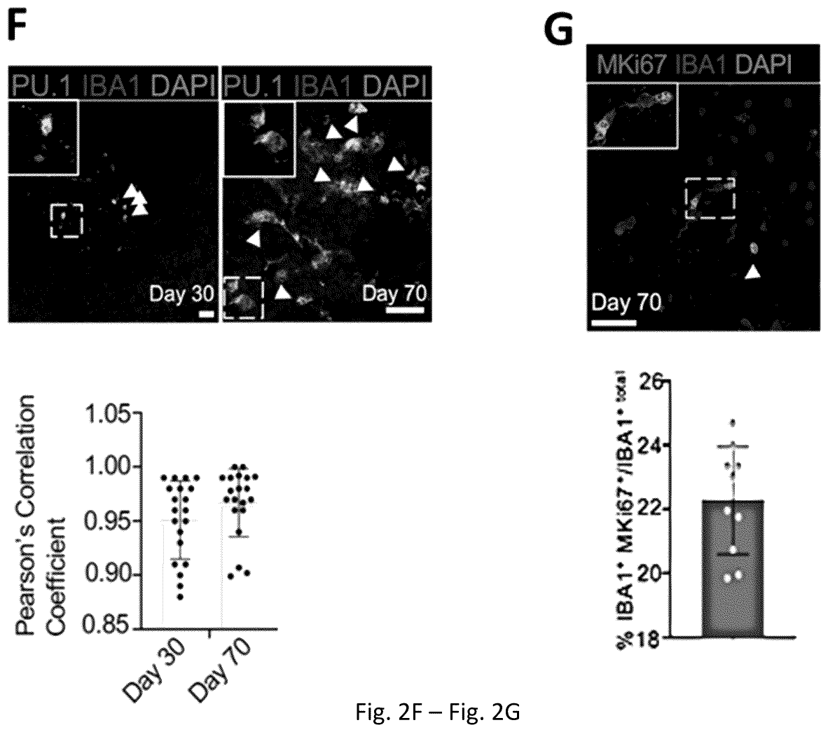

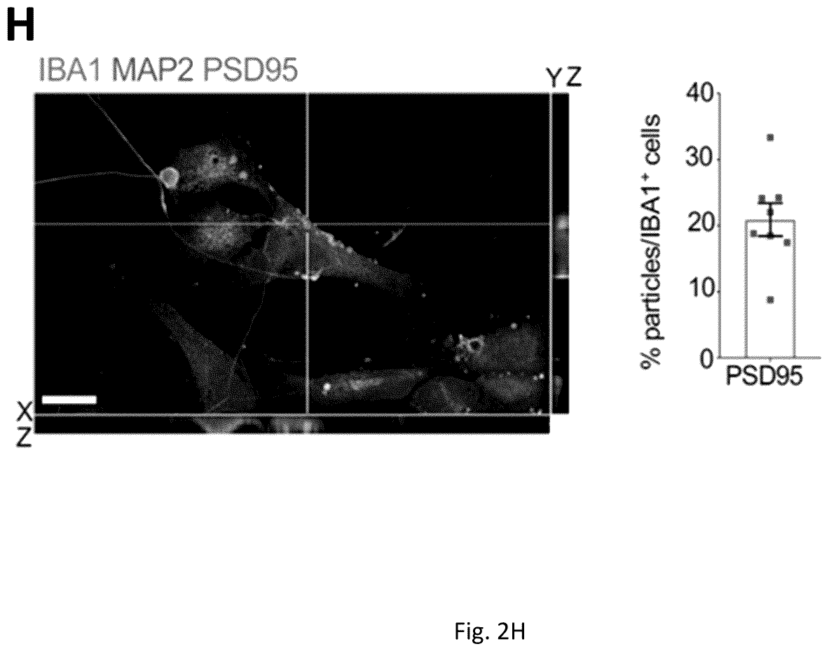

It was further tested whether overexpression of PU.1 in the developing cortical organoids could form myeloid cells that function as microglia. Since microglia account for 5-15% of mature brain cells (Perdiguero et al., 2016, Nat Immunol, 17:2-8; Schulz et al., 2012, Science, 336:86-90; Lawson et al., 1992, Neuroscience, 48:405-415), 10% of PU.1-infected BC4 hESCs were mixed with 90% parental hESCs to generate hCOs (FIG. 2A) (Xiang et al., 2017, Cell Stem Cell, 21:383-398 e387). In a previous attempt to incorporate endothelial cells (ECs) into hCOs, the partial induction of ETV2 at an early stage, followed by a full induction at day 18, was optimal in converting differentiating cells into ECs (Cakir et al., 2019, Nature Methods, 16:1169-1175). A similar induction scheme was applied in over-expressing PU.1 in hCOs (FIG. 2A). 3D live imaging of hCOs revealed the organization of the GFP⁺ cells that have an amoeboid cell shape at 30-day old hCOs (FIG. 1E). 70-day old hCOs contain a dramatically higher number of cells displaying the amoeboid cell shape than 30-day old hCOs (FIG. 1E). Consistent with the confocal imaging, PU.1-induced hCOs exhibit a higher expression of microglial markers at day−70 (FIG. 2B). Additionally, PU.1-induced hCOs showed increased expression of the microglial markers IBA1, CSF1R, CD11B, CX3CR1, TMEM119, and P2RY12 (FIG. 2B). These results suggest that hCOs with the forced PU.1 expression contain microglia-like cells. These organoids were named microglia-like cells-containing hCOs (mhCOs).

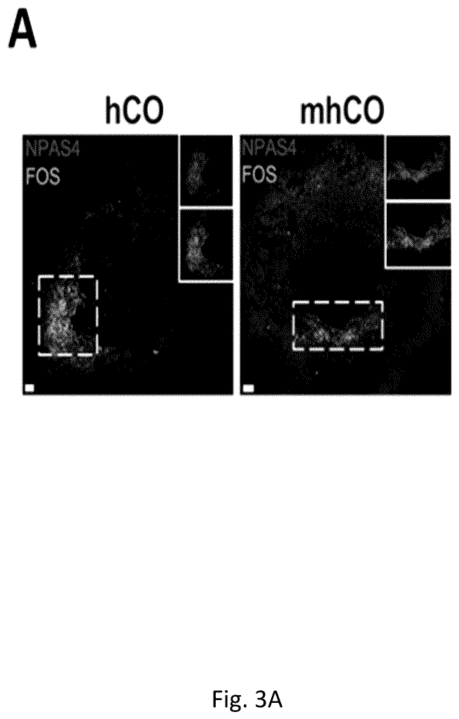

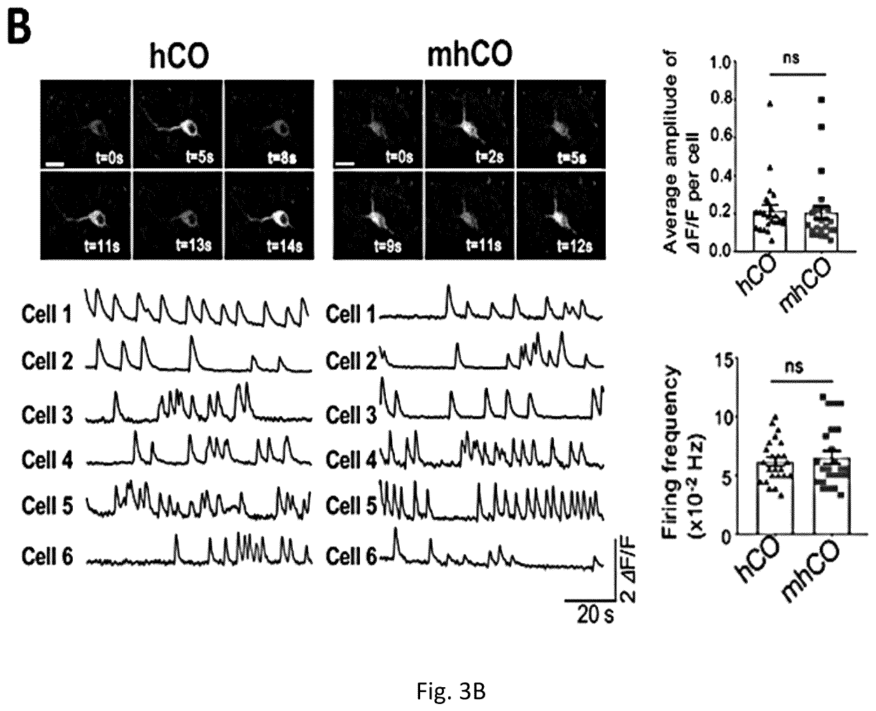

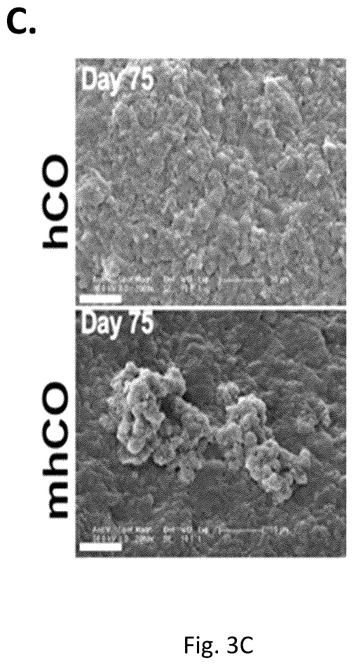

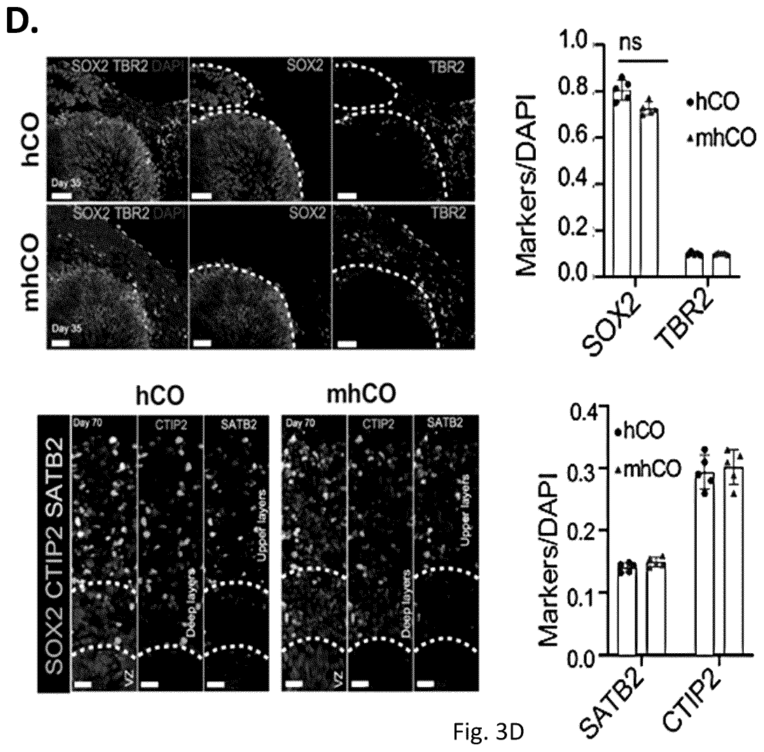

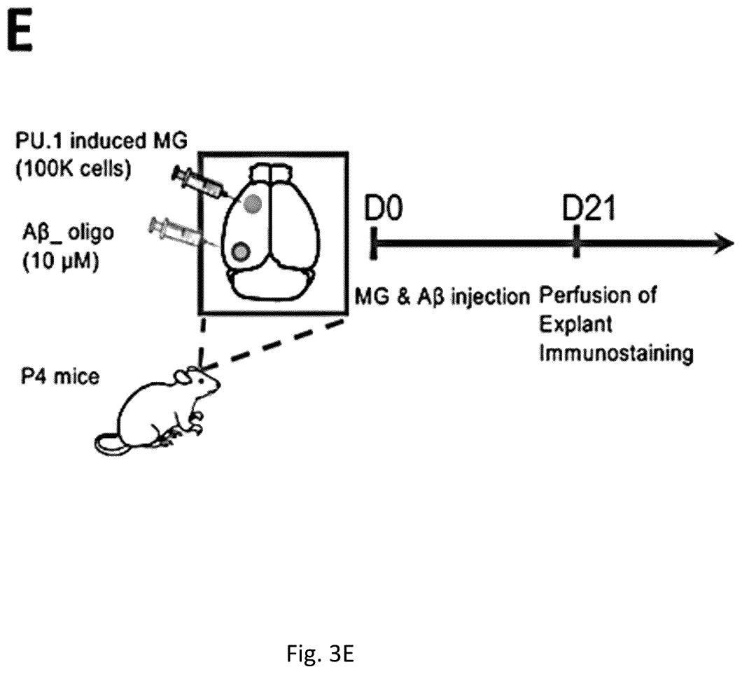

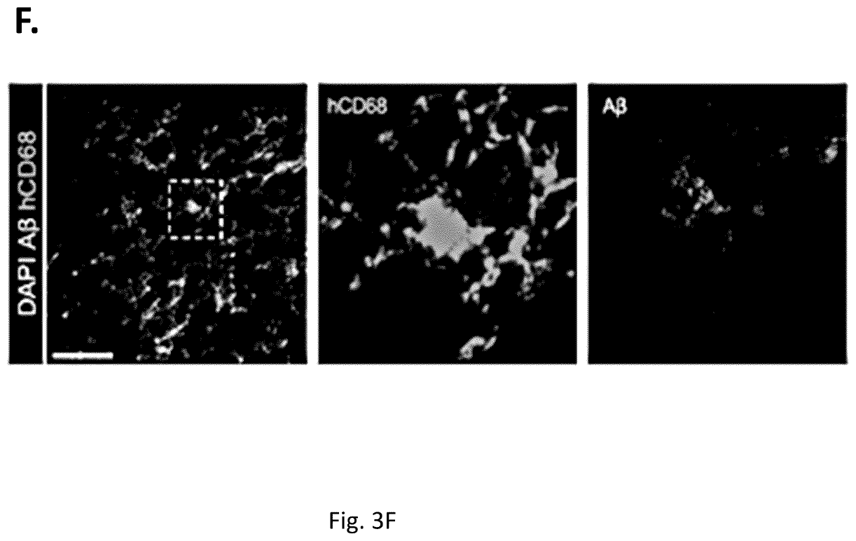

To investigate whether the microglia-like cells produced by PU.1-induction at mhCOs function like in vivo microglia, microglial characteristics were assessed in 30-, 70-, or 90-day old mhCOs. The immunostaining analysis for a microglia marker, IBA1, indicated the generation of microglia-like cells in mhCOs but not in control hCOs (FIG. 2C). On day 30, mhCOs contained many microglia-like cells with amoeboid morphology, whereas control hCOs lacked these cells. On day 70, more IBA1+ microglia-like cells were found in mhCOs, and they displayed an enhanced complexity in ramified morphology with the increased number of branch points characterized by Sholl analysis (FIG. 2C). qPCR results further supported the immunostaining data and showed an elevated expression of IBA1 in mhCOs compared to control hCOs at either day 30 or day 70 (FIG. 2B and FIG. 2C). As described previously (Guttikonda et al., 2021, Nat Neurosci, 24:343-354), GFP⁺ microglia-like cells were sorted and cultured with cortical neurons isolated from the same mhCOs at day 70 to characterize these immune cells in 2D- and 3D-culture settings. Co-immunostainings for IBA1 and CSF1R or TMEM119 and P2RY12 indicate that microglia-like cells acquire ramified structures within mhCOs as well as 2D-culture conditions (FIG. 2D and FIG. 2E). Even though GFP⁺ microglia-like cells became more ramified in 2D settings than 3D systems, mhCOs possess a higher expression of microglia-like cells P2RY12, suggesting the 3D-environment resembling the physiological conditions (FIG. 2B, FIG. 2D and FIG. 2E)²⁰. Notably, IBA1⁺ microglia in mhCOs are originated from PU.1 expressing GFP⁺ cells (FIG. 1F and FIG. 2F). On day 70, 22% of microglia-like cells express both IBA1 and MKI67, revealing the presence of proliferating microglia (FIG. 2G). The function of microglia on synapse pruning was next investigated. Indeed, co-immunostaining uncovered the presence of PSD95 puncta within IBA1+ microglia, indicating the presence of functional microglia engulfing or contacting the neuronal synaptic structures from mhCOs (FIG. 2H). Based on the detection of neuronal activity markers FOS and NPAS4 with single-molecule RNA fluorescent in situ hybridization, no difference in neuronal activity of mhCO and hCO was observed (FIG. 3A). It was further confirmed that both hCOs and mhCOs contained functional neurons with calcium surges utilizing the genetically encoded GCAMP6s (FIG. 3B). Moreover, no changes in either spontaneous firing amplitude or frequency were observed between hCOs and mhCOs, indicating that introduction of microglia-like cells does not influence the functional properties of neurons in organoids (FIG. 3B). The presence of microglia-like cells on the surface of mhCOs but not on the surface of hCOs was noted by using scanning electron microscopy (FIG. 3C). Regarding corticogenesis, no significant difference in the formation of cortical layers was noted in mhCOs compared to hCOs, as demonstrated by the presence of the intermediate progenitors, deeper and upper cortical layer neurons characterized by the expression of TBR2, CTIP2, or SATB2 (FIG. 3D). These data suggest that the generation of microglia-like cells by PU.1-induction has little impact on cortical development. Collectively, PU.1 induction directs cells into microglia-like cells in hCOs.

PU.1 Induced Microglia-Like Cells from hESCs Engrafting Mouse Brain.

The in vivo function of PU.1-induced microglia-like cells in the mouse brain was next examined. Microglia-like cells from hESCs by PU.1 induction were transplanted into the brain of an immune-deficient mouse and treated with Aβ-oligo (FIG. 3E). After 3-weeks of transplantation, immunostaining for human-specific CD68 and Aβ revealed that PU.1-derived microglia-like cells displayed a complex ramified morphology and were functionally active, responding to oligomeric Aβ (FIG. 3F). Collectively, these data suggest that microglia-like cells from PU.1 induction function similarly within the mouse brain environment and human brain organoids.

A Single-Cell Map of Developing mhCOs Reveals the Presence of Microglia-Like Cells Resembling the Primary Counterparts.

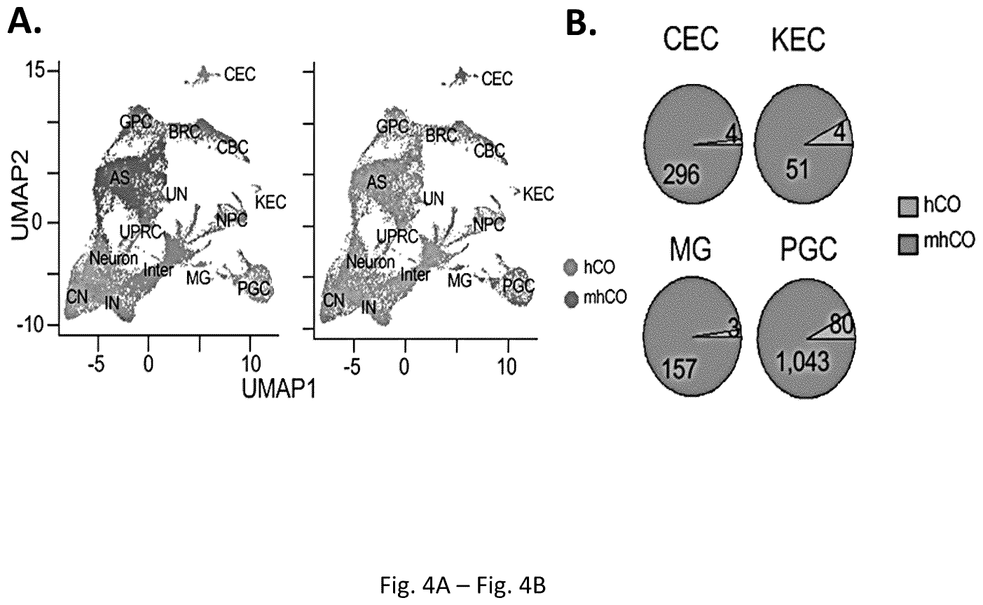

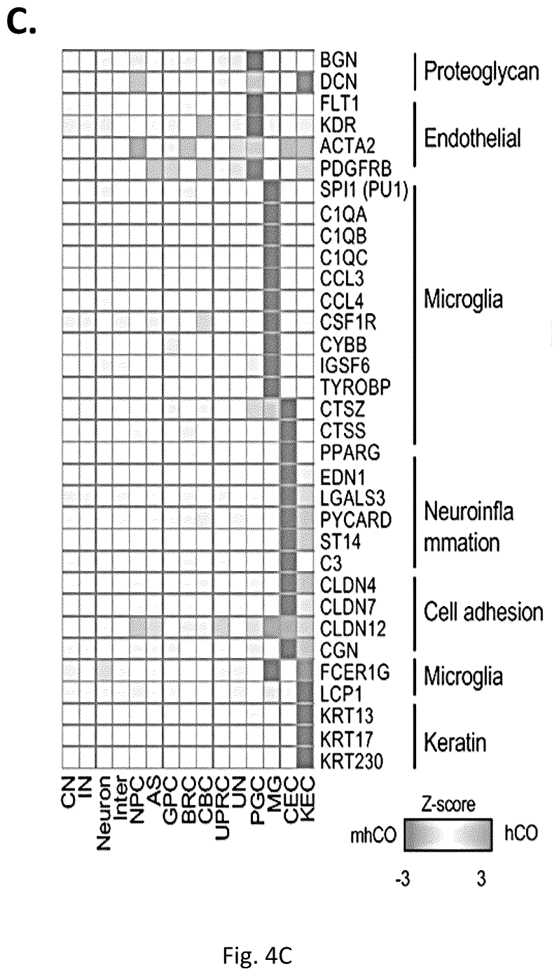

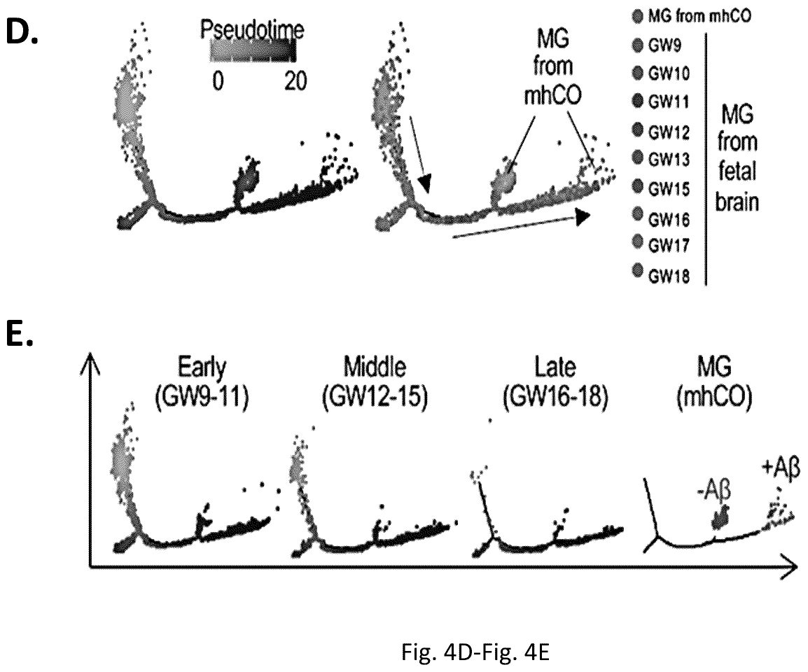

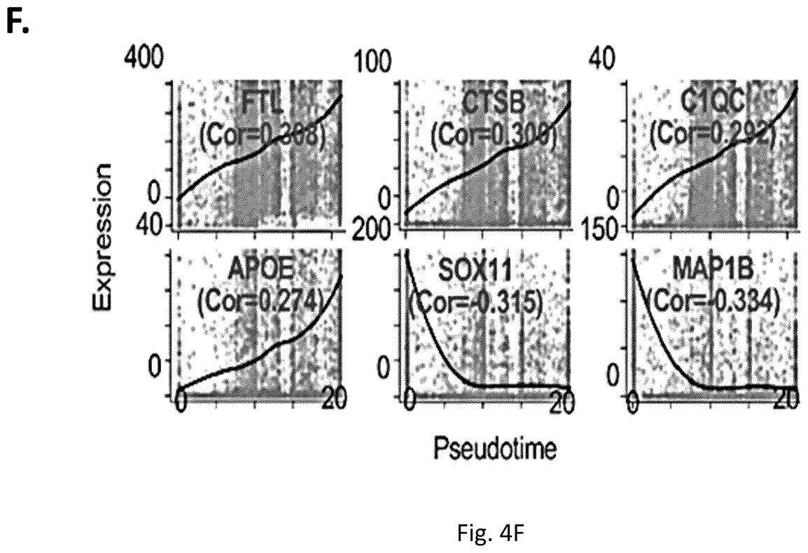

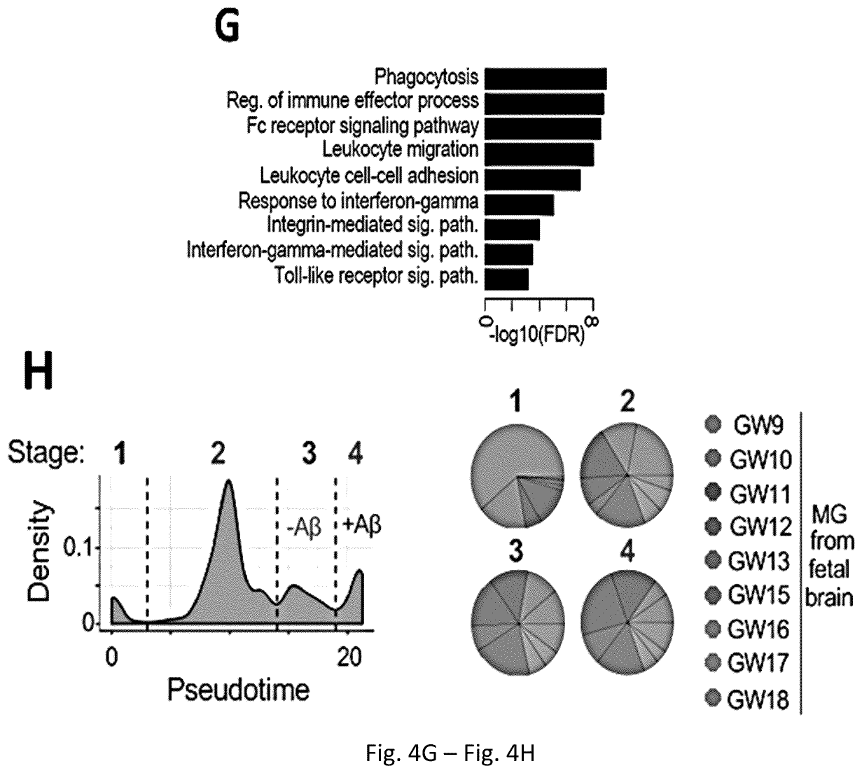

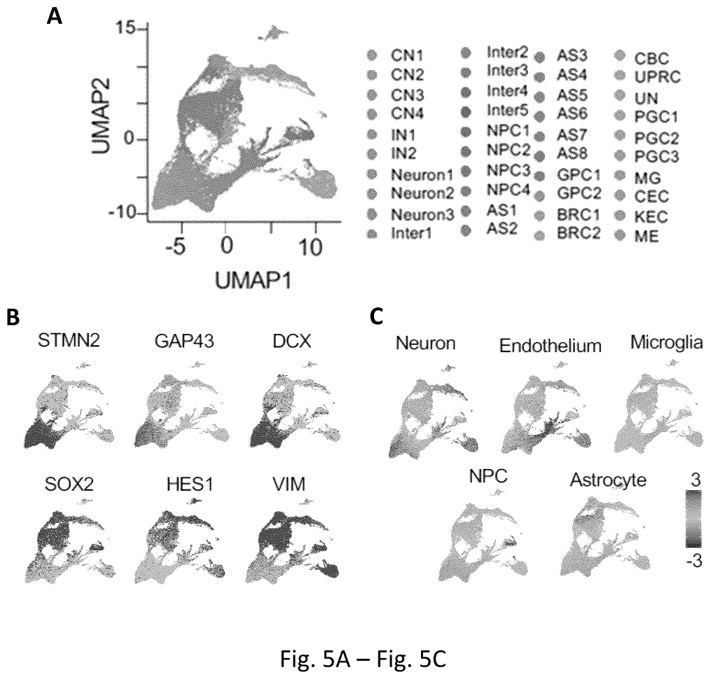

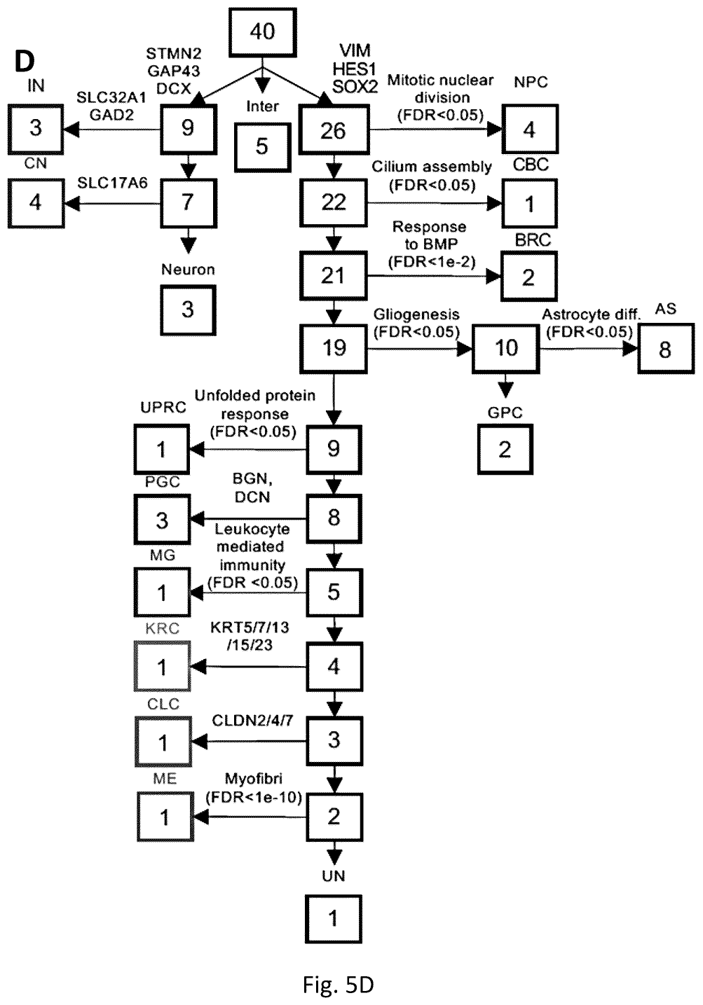

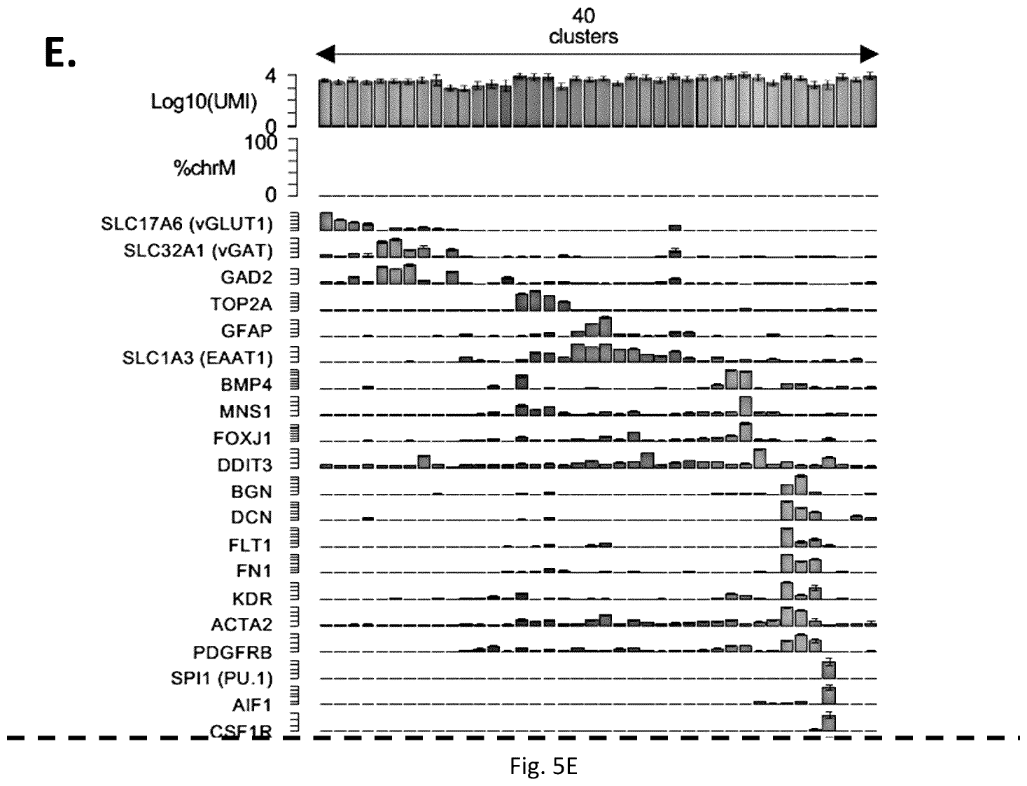

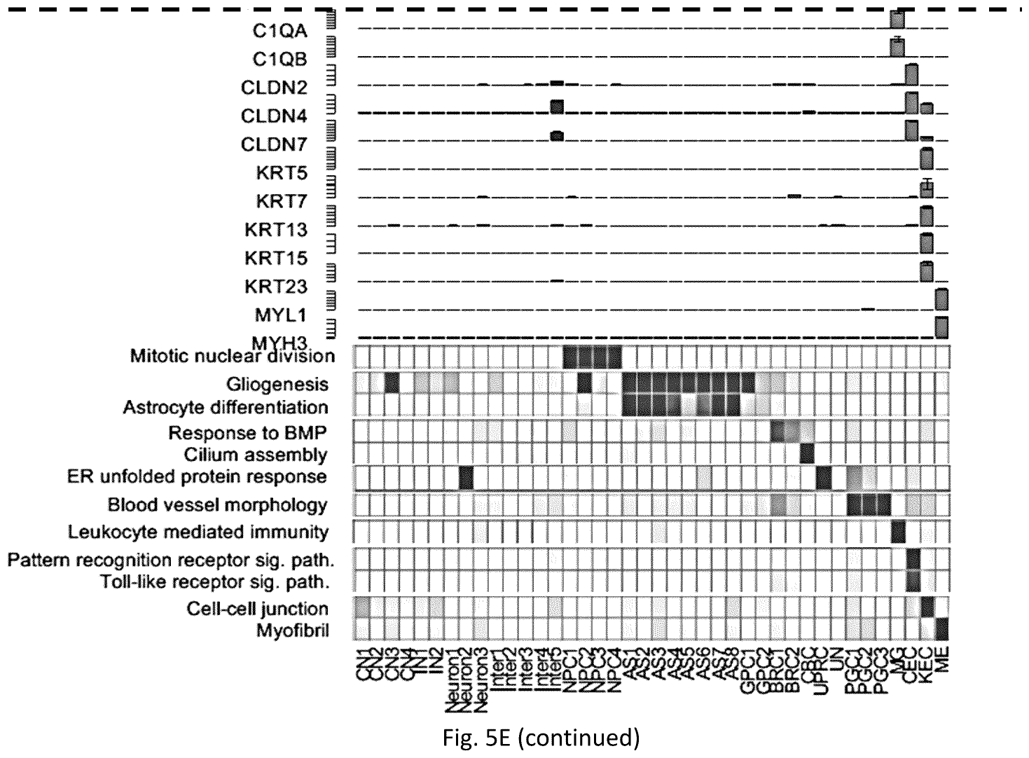

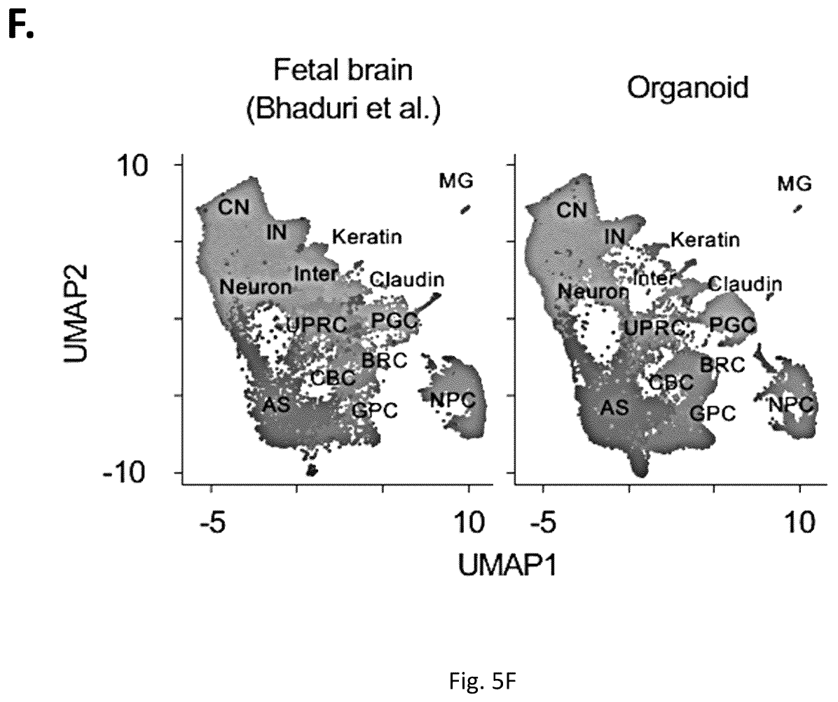

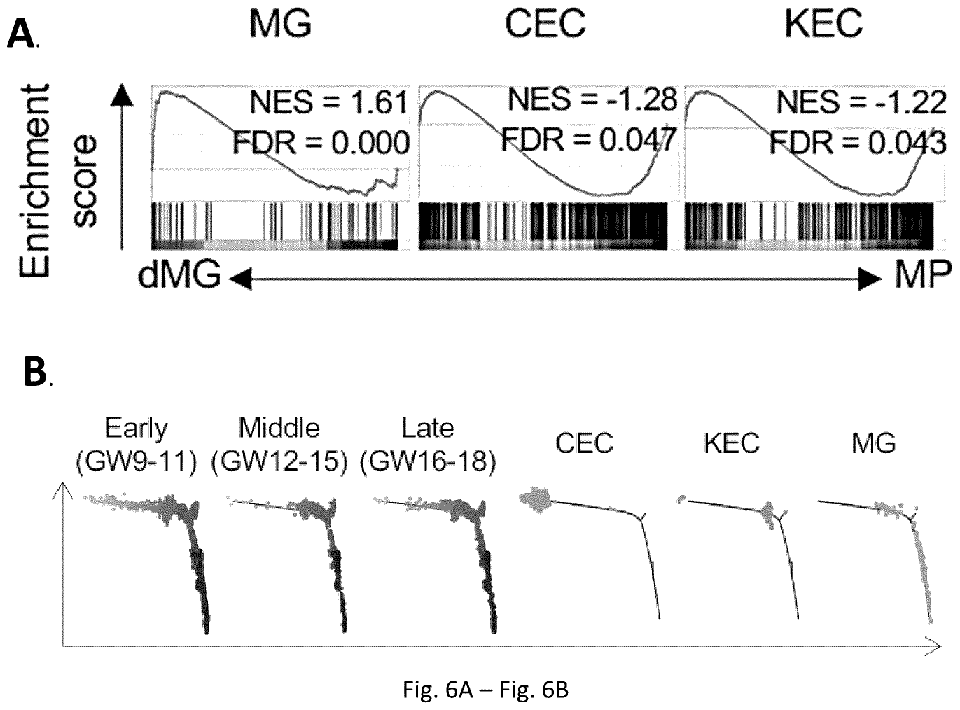

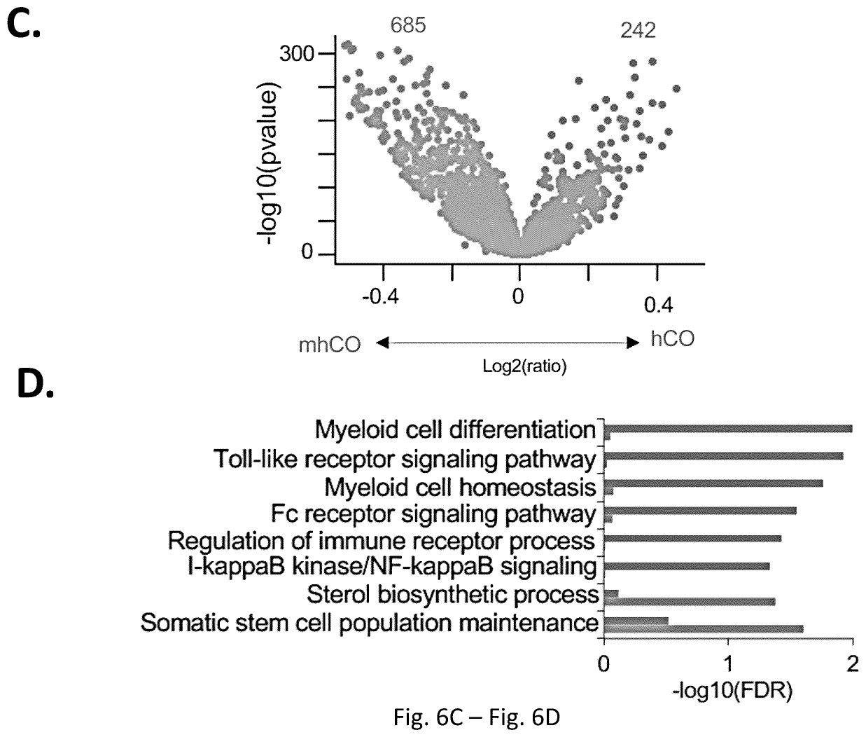

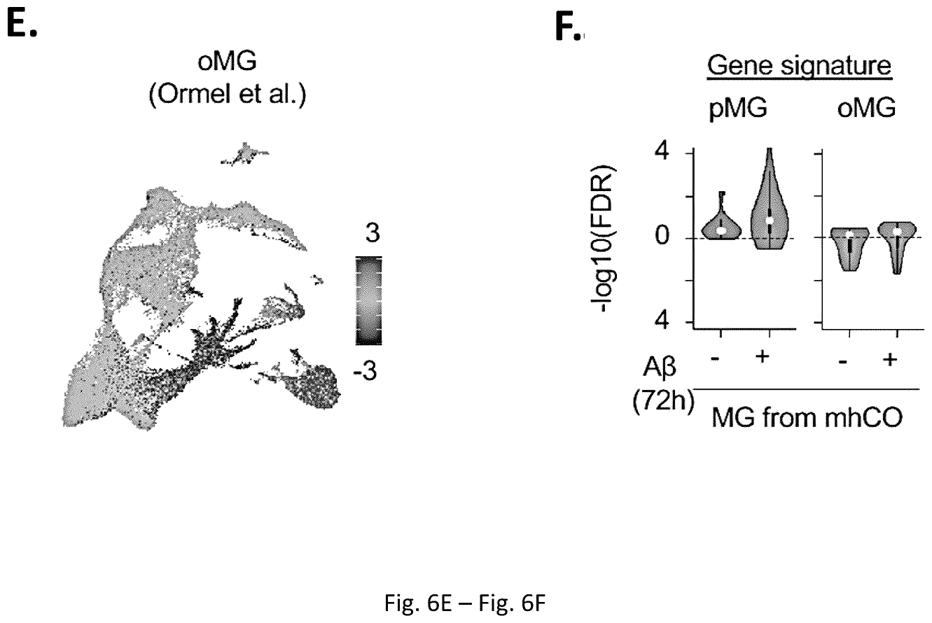

Single-cell RNA-sequencing (scRNA-seq) was performed in the 10× Genomics platform to examine the lineage specification employed by PU.1 induction during cortical organoid development. After quality control, a total of 13,416 and 13,546 cells were analyzed from hCOs and mhCOs, respectively (FIG. 4A). Cell clusters were then systematically assigned to 14 cell types, including cortical neurons, interneurons, astrocytes, and other previously-defined cell types (FIG. 5) (Xiang et al., 2019, Cell Stem Cell, 24:487-497 e487). Interestingly, mhCOs uniquely produced four cell types, including microglia cluster (MG) with high expression of the complement/chemokine system (e.g., C1QA, C1QC, CCL3/4), PGC cluster highly expressing proteoglycans, CEC cluster expressing tight junction proteins (claudin, CLDN4/7/12), and KEC cluster expressing cytofilament proteins (keratin, KRT13/17/19) (FIG. 4A and FIG. 4B). Comparative analysis with the reference transcriptome and unique cell markers validated that the cell cluster with the immune system genes is microglia (FIG. 5C and FIG. 5E)(Darmanis et al., 2015 Proc Natl Acad Sci USA, 112:7285-7290). PGCs were previously shown to transdifferentiate into endothelial cells by a defined transcription factor, ETV2 (Cakir et al., 2019, Nature Methods, 16:1169-1175). In mhCO-derived cells, enhanced expression of endothelial (FLT1 and KDR) and pericyte markers (ACTA2 and PDGFRB) were also detected in PGC (FIG. 4C). This data suggests that PU.1 promotes mesoderm-derived cell types. Claudin- (CEC) and keratin-enriched cells (KEC) clusters also expressed some of the microglia-related genes (e.g., CTSS, LCP1) (FIG. 4C). Notably, both cell types are also detected in single-cell transcriptome from in vivo human fetal brain samples (FIG. 5F) (Bhaduri et al., 2020, Nature, 578:142-148). Their gene signatures are significantly enriched in in vitro-derived microglia precursors, suggesting that these cell types are potential precursors of microglia cells (FIG. 6A) (Svoboda et al., 2019, Proc Natl Acad Sci USA, 116:25293-25303). A cell trajectory analysis of CEC, KEC, and MG clusters was performed with human fetal microglia (Kracht et al., 2020, Science, 369:530-537). Pseudotime calculation indicates that the CEC cluster is the earliest stage during human fetal microglia development (FIG. 6B). Cells showing similar transcriptome with CEC are detected in early gestation week (GW9-11) but rare in the middle and late stages. KEC-like cells are noted in all gestation weeks, but earlier stage than MG cluster (FIG. 6B). Thus, these results support the hypothesis that CEC and KEC are potential precursors during microglia development.

Comparative analysis of the global gene expression profile revealed that mhCOs displayed a significant induction of genes involved in immune cell differentiation, toll-like receptor signaling, and NF-κB signaling (FIG. 6C and FIG. 6D). Some of these genes are also related to inflammatory signaling pathways indicating that microglia within mhCOs responding to the internal stress, most likely apoptotic cells due to the limited diffusion of nutrients and oxygens at day 90 organoids (Pasca et al., 2018, Nature, 553:437-445; Lancaster et al., 2014, Science 345, 1247125). These results indicate that PU.1 mediates the lineage commitment into microglia-like cells during cortical organoid development.

In one of the previous studies by Ormel et al., authors detected the microglia-like cells, defined as oMG (organoid MG), in brain organoids made by an unguided protocol (Ormel et al., 2018, Nat Commun, 9:4167). The enrichment of gene signatures of oMG and primary microglia (pMG) was applied to the scRNA-seq data to examine differences between the innately developed oMG and the PU.1-induced microglia (MG). It was found that oMG gene signatures were enriched in PGC clusters but depleted in MG clusters (FIG. 6E and FIG. 6F). In contrast, pMG gene signatures were enriched in MG clusters and significantly increased with Aβ treatment (FIG. 6F, p=4.70-e2 by two-sided t-test). These results suggest that PU.1-induction produced microglia-like cells demonstrating a more similar transcriptome profile to primary microglia than oMG.

In the embryonic and early postnatal brain, microglia are heterogeneous and progressively mature (Kracht et al., 2020, Science, 369:530-537). Integrative analysis of scRNA-seq revealed that transcriptional profiles of microglia-like cells from mhCOs are similar to those in the middle and late stages of fetal microglia development (FIG. 4D). Assigned pseudotimes are increased with the gestational week (GW) (FIG. 4E) and orchestrated with activation of immune sensing (FIG. 4F and FIG. 4G). The pseudotime trajectory analysis classified microglia into four distinct developmental stages (FIG. 4H). The PU.1 induction in the brain organoid produced the third stage of microglia-like cells mainly composed of GW12-18 (FIG. 4H and FIG. 4I). Collectively, the present results indicate that microglia-like cells in mhCOs recapitulate the transcriptional surveillance of the human fetal brain to pathological hallmarks.

Lineage Commitment of PU.1-eGFP Expressing Cells and their Functions in mhCOs.

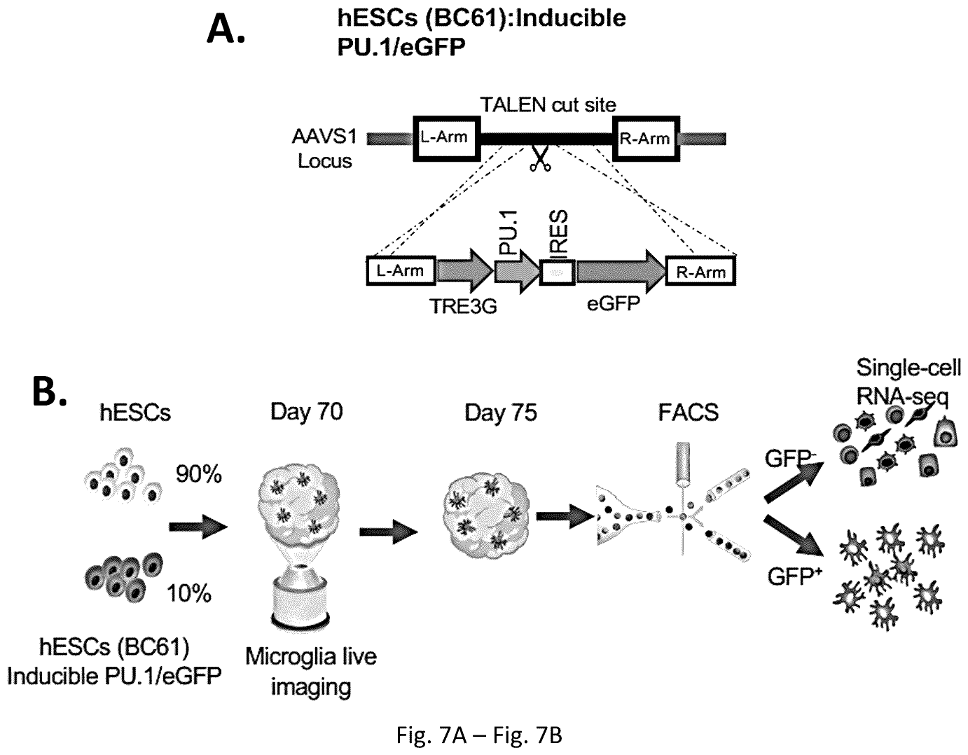

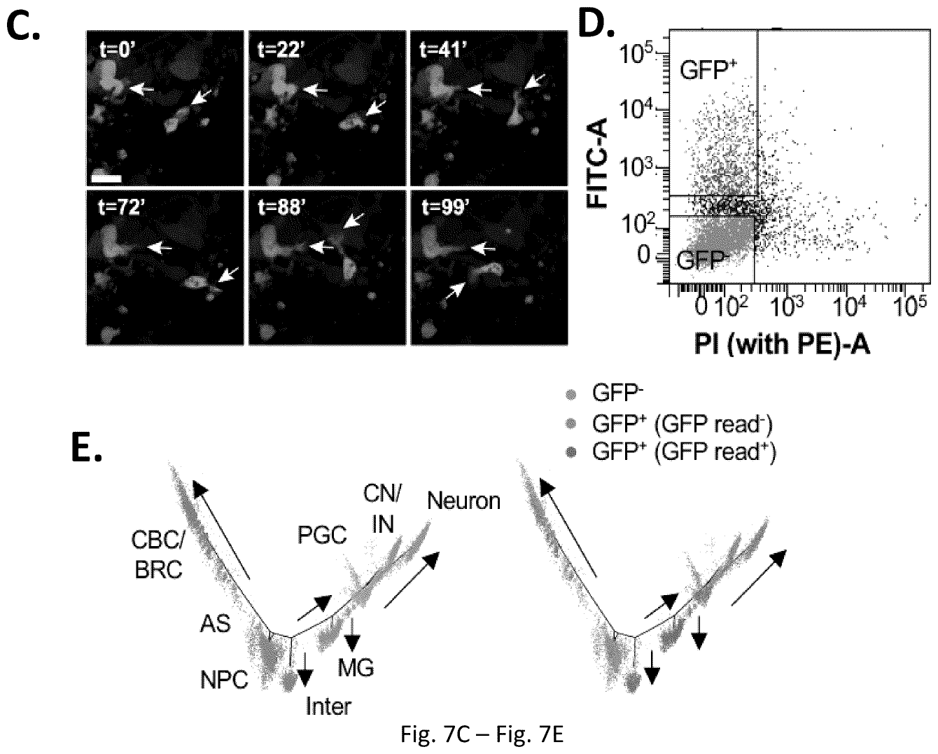

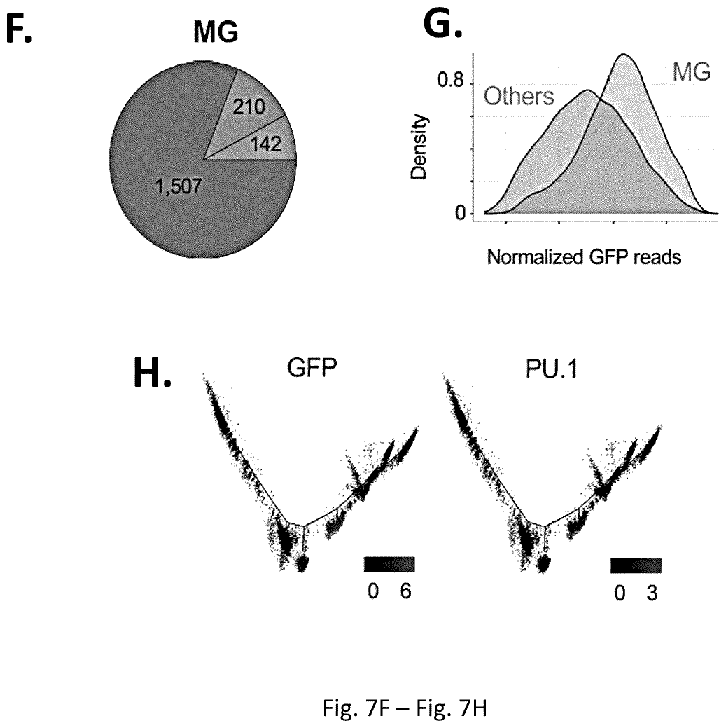

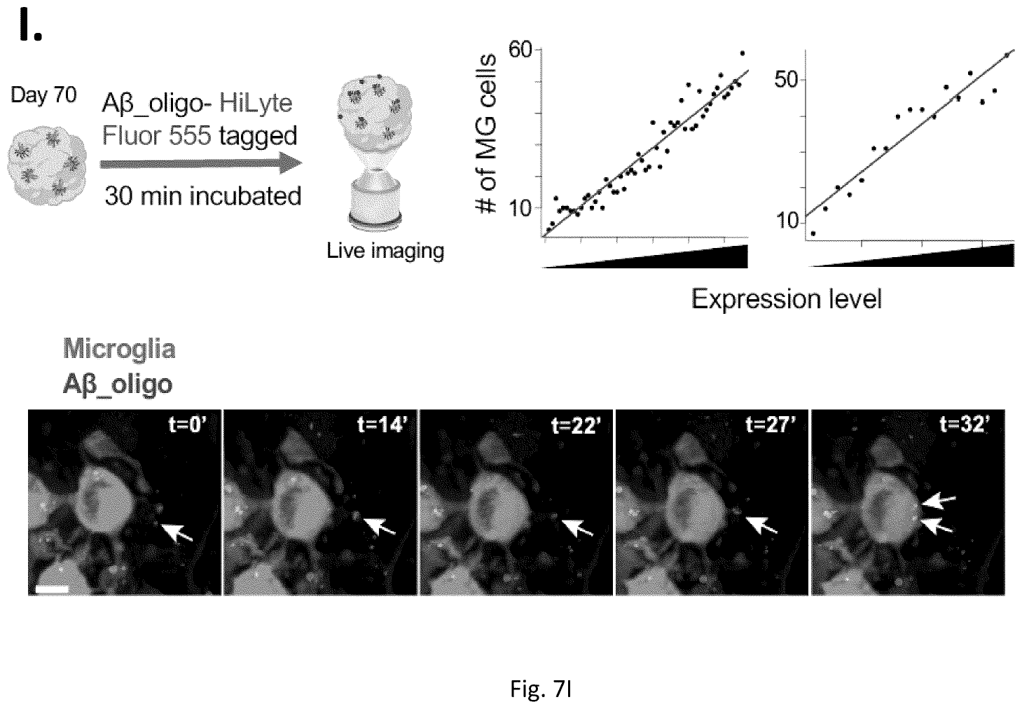

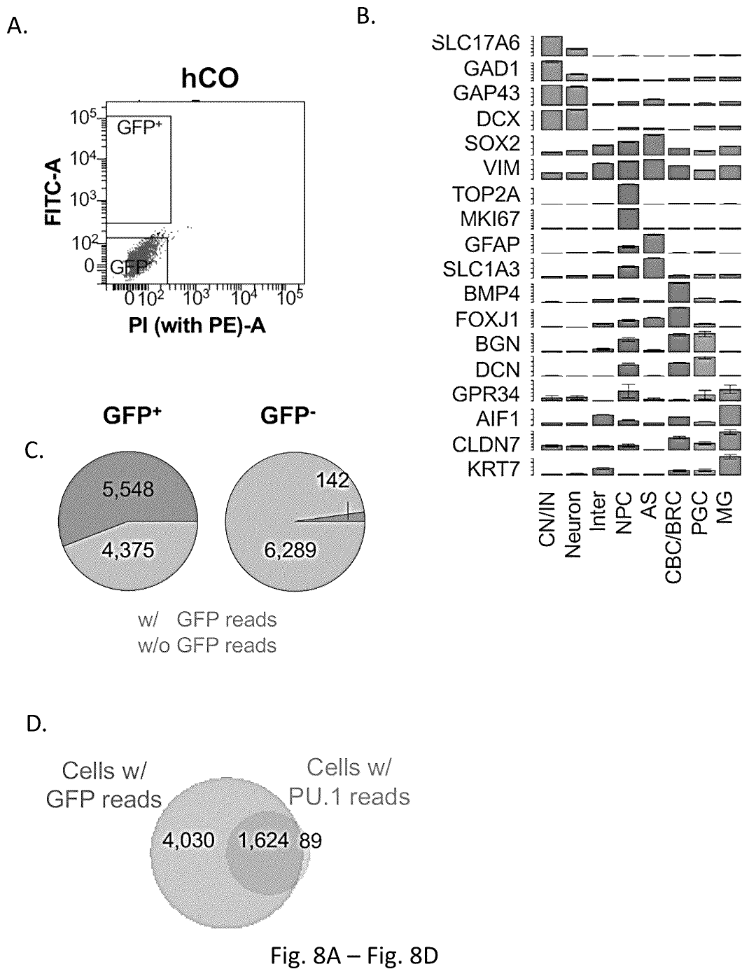

To address the characterization of heterogeneity of the cells produced via PU.1 induction within mhCOs, dox-inducible PU.1-iRES-eGFP and rtTA cassette were introduced into the AAVS1 safe locus to generate hESC line (BC61) with robust transgene expression tagged with GFP (FIG. 7A). Cortical organoids with microglia-like cells (mhCO), as described above, mixing 10% of BC61 hESC cells with 90% parental hESC cells and characterized GFP⁺ microglia-like cells via live-imaging (FIG. 7B). Time-lapse imaging experiments up to 2-hours showed that GFP+ microglia-like cells acquire diverse morphology, amoeboid and ramified, and remarkably motile and dynamic (FIG. 7C). GFP⁺ and GFP⁻ cells were extracted by fluorescence-activated cell sorting on day 75 to examine how GFP-expressing cells committed into myeloid lineage via performing scRNA-seq (FIG. 7B, FIG. 7D and FIG. 8A). The developmental trajectory segregated cells into eight different branches corresponding to microglia, neurons, astrocytes, and other cell lineages (FIG. 7E and FIG. 8B). It is noted that more than half of cells in GFP+ populations contain GFP-derived RNA-seq reads, whereas the GFP-derived reads were rarely detected in GFP− cells (FIG. 8C). While not wishing to be bound by any particular theory, it is supposed that GFP+ cells without GFP-derived reads may induce the transgene with a lower level than those with GFP-derived reads. Notably, most cells in the MG branch were composed of cells from the GFP⁺ population with GFP-derived reads (FIG. 7E and FIG. 7F). GFP expression in the MG branch is significantly higher than that in other branches (FIG. 7G). Though some GFP⁺ cells with GFP-derived reads were classified into different cell types (e.g., intermediate), GFP⁻ cells and GFP⁺ cells without GFP-derived reads were barely differentiated into MG lineage. Since exogenous PU.1 does not contain poly-A tail, PU.1 reads are mainly derived from endogenous PU.1, usually silenced in non-MG cell types in the brain. It was found that the vast majority of PU.1-expressing cells contain GFP-derived reads (FIG. 8D), and endogenous PU.1 expression level was positively correlated with GFP expression (Spearman correlation=0.520). Interestingly, cells with higher GFP and endogenous PU.1 levels are more likely to be differentiated into microglia (FIG. 7H). Overall, the present results suggest that the substantial expression of PU.1 assists microglia fate specification in the organoid.

It was next examined whether GFP+ microglia-like cells display phagocytotic functions by performing 4D (x, y, z, and t) imaging of live mhCO. To visualize the response of microglia towards Aβ₁₋₄₂-oligo inflammation, mhCOs were treated with 1 μM of Aβ₁₋₄₂-oligo-(HiLyte)-555 for 30 minutes (FIG. 7I). Live imaging of GFP⁺ microglia-like cells and Aβ₁₋₄₂-oligo-(HiLyte)-555 was performed and it was observed that several GFP⁺ microglia-like cells already captured Aβ₁₋₄₂-oligo-(HiLyte)-555 found in the soma of microglia. As a particular event, microglia-like cells migrated toward Aβ, extended processes, and captured Aβ for phagocytosis lasting in around 30 minutes (FIG. 7I). Thus, GFP⁺ microglia-like cells rapidly process Aβ in mhCOs resembling quick (in minutes) microglial actions towards Aβ in mice brain (Condello et al., 2015, Nature Communications, 6:6176). Taken together, PU.1 drives the generation of microglia-like cells with high motility and functionality in mhCOs.

Microglia within mhCOs Recapitulates the Actions of Primary Microglia Towards Amyloid-β

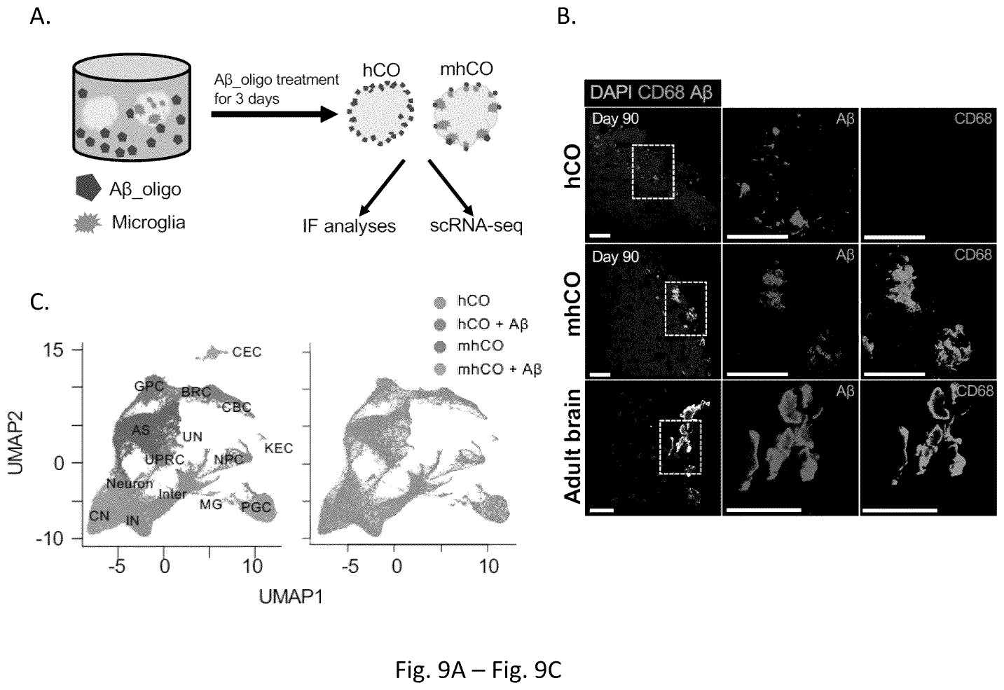

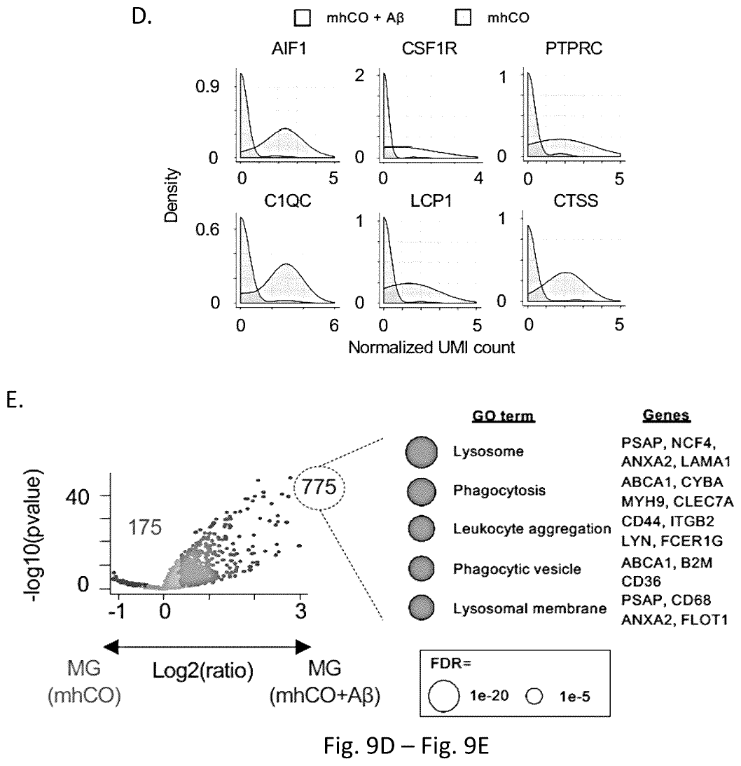

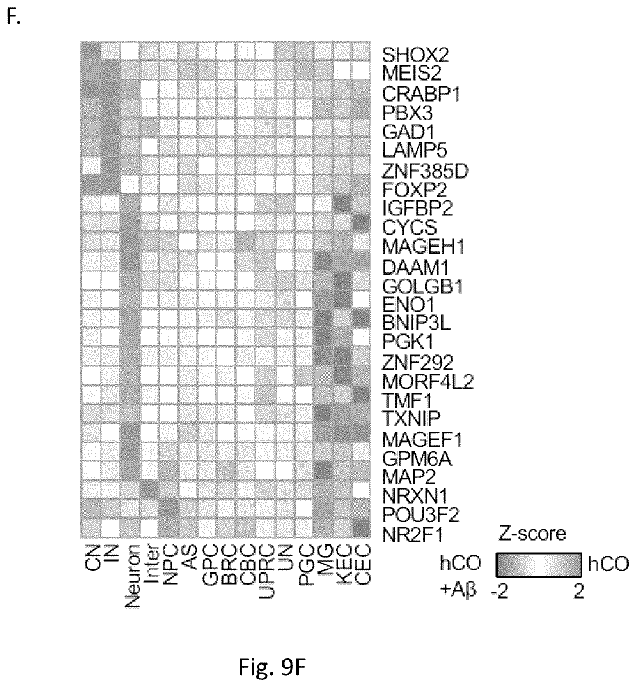

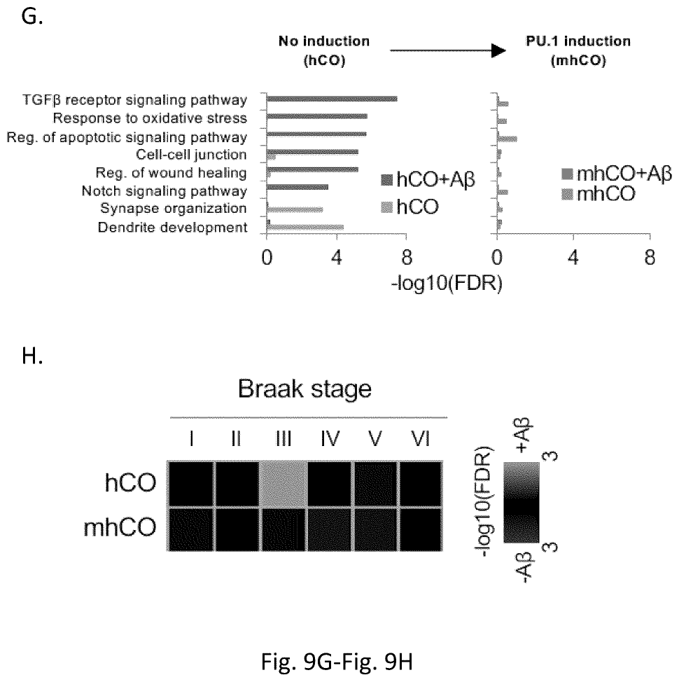

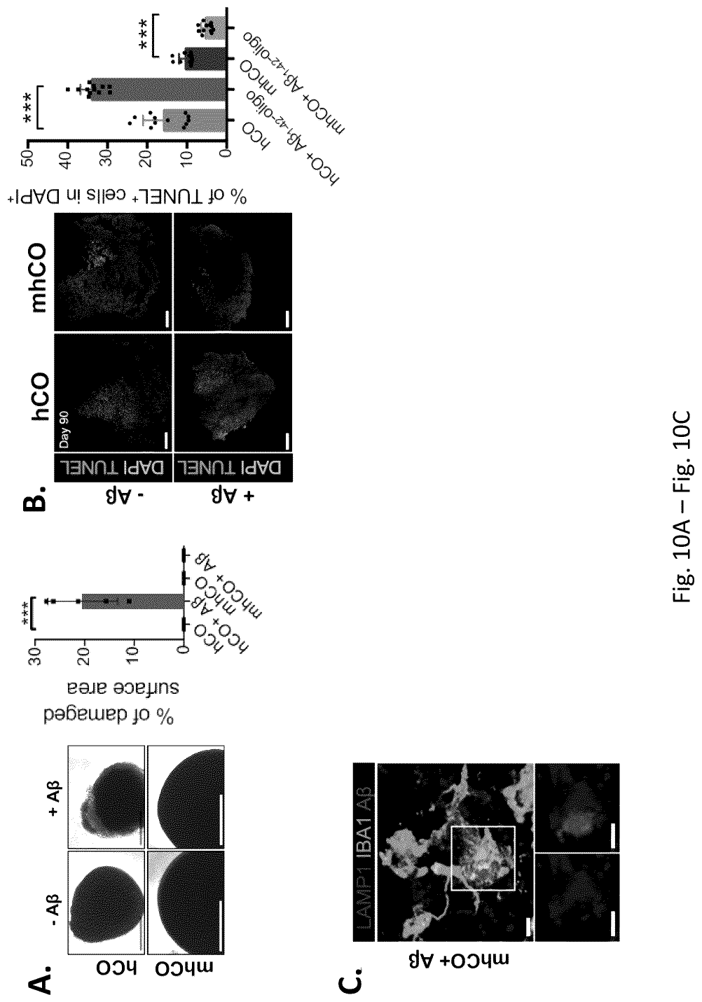

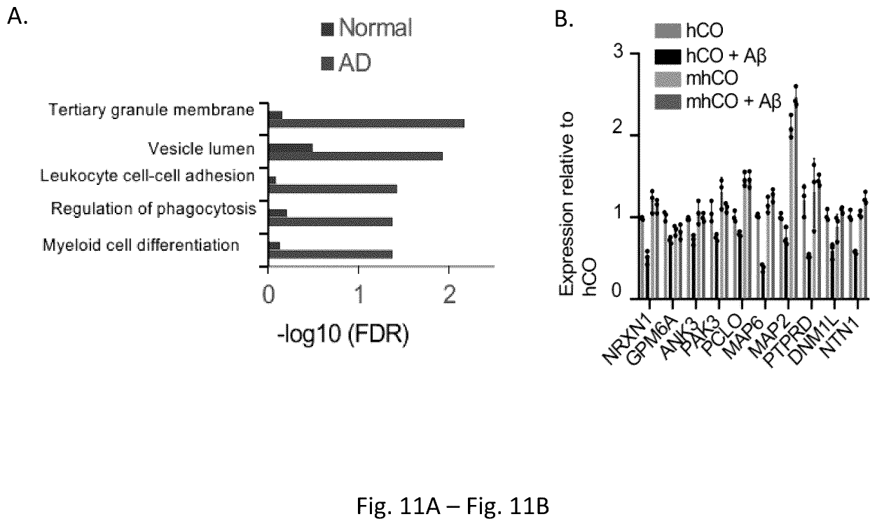

Microglia play critical roles in inflammation within the CNS, such as a rapid response to inflammatory signaling (Salter et al., 2017, Nat Med, 23:1018-1027). The function of microglia under Aβ₁₋₄₂-oligo treatment (for 72 h), a pro-inflammatory trigger (FIG. 9A) was investigated. The Aβ₁₋₄₂-oligo treatment led to a dramatic change in the surface of control hCOs. Surprisingly, Aβ₁₋₄₂-oligo had little effect on the surface of mhCOs (FIG. 10A). Apoptosis in hCOs induced by Aβ₁₋₄₂-oligo was prominent as represented by deterioration on the exterior of hCOs, while mhCOs exhibited reduced apoptotic areas on their surfaces (FIG. 10A). Notably, mhCOs treated with Aβ₁₋₄₂-oligo displayed even fewer apoptotic areas than non-treated mhCOs, suggesting that Aβ₁₋₄₂-oligo further enhanced the scavenging function of the microglia-like cells in mhCOs (FIG. 10B). Surprisingly decreased apoptotic regions in mhCOs under Aβ-treatment indicate a regular or extensive activation of microglia cells. Though the number of microglia is limited in mhCOs, the incubation of Aβ₁₋₄₂-oligo for 3 days may keep them fully activated and direct them to eliminate apoptotic cells. Co-staining of Aβ with CD68 demonstrated a co-localization of microglial phagocytotic markers with Aβ oligos (FIG. 9B). Indeed, staining for IBA1, LAMP1 and Aβ revealed that dystrophic neurites marked by LAMP1 surrounded Aβ oligos (FIG. 10C). Notably, microglia frequently engage with Aβ oligos which may show the protective role of microglia on Aβ induced neurotoxicity (FIG. 10C). Similar to actions of microglia towards Aβ in mhCOs, primary microglia in human tissue engulf the Aβ, characterized by the presence of CD68 (FIG. 9B). The microglia in mhCO and primary human tissue showed that microglia-like cells have processes different from typical microglia surrounding Aβ plaques (Condello et al., 2015, Nature Communications, 6:6176). The difference may be due to the in vitro response different from the in vivo condition (Bard et al., 2000, Nat Med, 6:916-919). These results suggest that microglia-like cells within mhCOs become activated, engulf the Aβ, and scavenge the dead cells.

### A Single-Cell Map of Amyloid-O Treated Organoids Uncovers the Protective Role of Microglia.

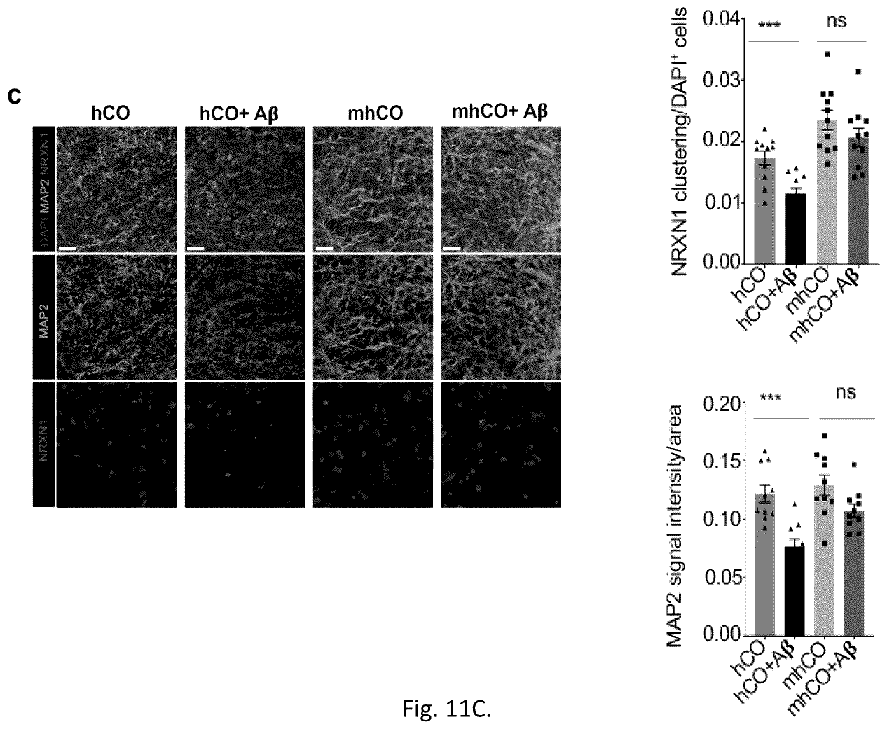

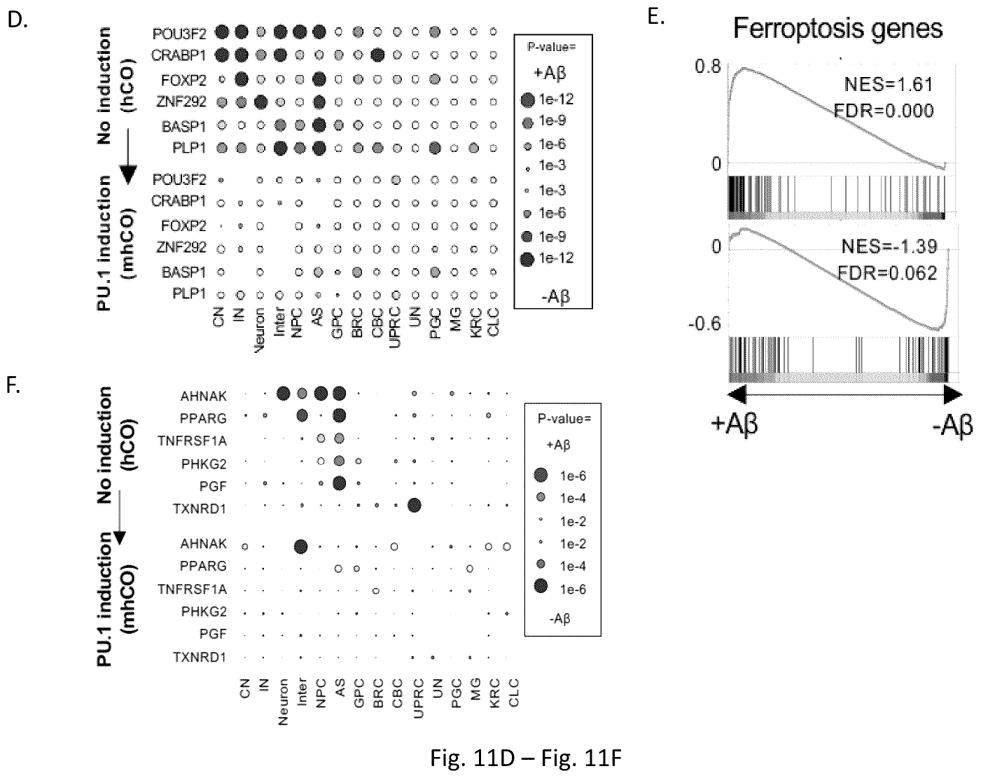

To examine the effects of Aβ on cell differentiation and transcriptional program in hCOs, the single-cell transcriptome was investigated in cortical organoids treated with Aβ (FIG. 9C). Aβ-treated mhCOs exhibited a significant up-regulation of genes associated with microglial activation (AIF1, C1QC, CSF1R, LCP1, PTPRC, and CTSS) in MG clusters compared to those in non-treated mhCOs (FIG. 9D). Furthermore, as observed in the brain of an AD mouse model (Keren-Shaul et al., 2017, Cell, 169:1276-1290 e1217), microglia-like cells in Aβ-treated mhCOs showed a high expression of genes involved in the lysosome, phagocytosis, and leukocyte aggregation (FIG. 9E). Thus Aβ treatment led to the induced phagocytic activity of microglia in the organoids. The induction of phagocytic genes was also observed in the AD patient brain compared to the brain from healthy donors (FIG. 11A) (Grubman et al., 2019, Nat Neurosci, 22:2087-2097).

In addition to mhCOs, the effects of Aβ-treatment on hCOs was also evaluated (FIG. 9C). Global transcriptome comparison revealed that the genes related to synapse and dendrite development were significantly downregulated (FIG. 9F). qPCR and immunostaining analyses further confirmed the dysregulation of synaptic and dendritic genes in Aβ-treated hCOs but not in Aβ-treated mhCOs (FIG. 11B and FIG. 11C). hCOs showed the abnormal induction of genes for apoptotic, TGFβ, and Notch signaling (FIG. 9G) involved in AD pathogenesis (Barinaga et al., 1998, Science, 281:1303-1304; Wyss-Coray et al., 1997, Nature, 389:603-606). Significantly, the transcriptional dysregulation of dendritic and synapse development and apoptotic genes was significantly attenuated in mhCOs (FIG. 9G). Moreover, genes promoting neuronal differentiation and maturation (BRN2 (POU3F2), CRABP1, and FOXP2) as well as glia cell commitment (BASP1 and PLP1) were drastically reduced in hCOs but were protected in mhCOs (FIG. 11D). These results suggest that functional microglia in mhCOs rescues the Aβ-mediated cellular and molecular abnormalities in hCOs.

Ferroptosis is a type of cell death caused by iron-dependent lipid peroxidation. Increased iron level is a common feature of neurodegeneration and is related to the progression of AD (Abdalkader et al., 2018, Front Neurosci, 12:466). Notably, Aβ treatment resulted in the over-expression of ferroptosis mediators, such as PHKG2 (Yang et al., 2016, Proc Natl Acad Sci USA, 113:E4966-4975), TXNRD1 (Malhotra et al., 2010, Nucleic Acids Res, 38:5718-5734), and PPARG (Abdalkader et al., 2018, Front Neurosci, 12:466), in hCOs but not in mhCOs (FIG. 11E). GSEA further confirmed that ferroptosis-associated genes were significantly increased with Aβ treatment in hCOs (FIG. 11F). Thus, the presence of functional microglia attenuates Aβ-mediated induction of ferroptosis. The transcriptome profiles of the Aβ-treated cortical organoids was further compared with those from AD patient cortexes collected at different Braak tangle stages (Li et al., 2019, Nat Commun, 10:2246). Comparison analysis revealed that Aβ-treated hCOs displayed a significant enrichment of gene signatures of stage III, during which the neurofibrillary lesions invade the neocortex region and patients start to exhibit the clinical symptoms (FIG. 9H) (Braak et al., 2006, Acta Neuropathol, 112:389-404). On the contrary, mhCOs did not show any stage-specific gene signatures (FIG. 9H). Interestingly, Aβ treatment induced the microglia into the fourth stage of fetal microglia that displays transcriptional features of phagocytic microglia (FIG. 4H). Overall, the Aβ-treated brain organoids recapitulate the onset of AD-associated transcriptomic profile, which can be rescued by PU.1-induced microglia-like cells.

mhCOs Provide a Valuable Tool to Examine the Role of AD-Associated Microglia Genes.

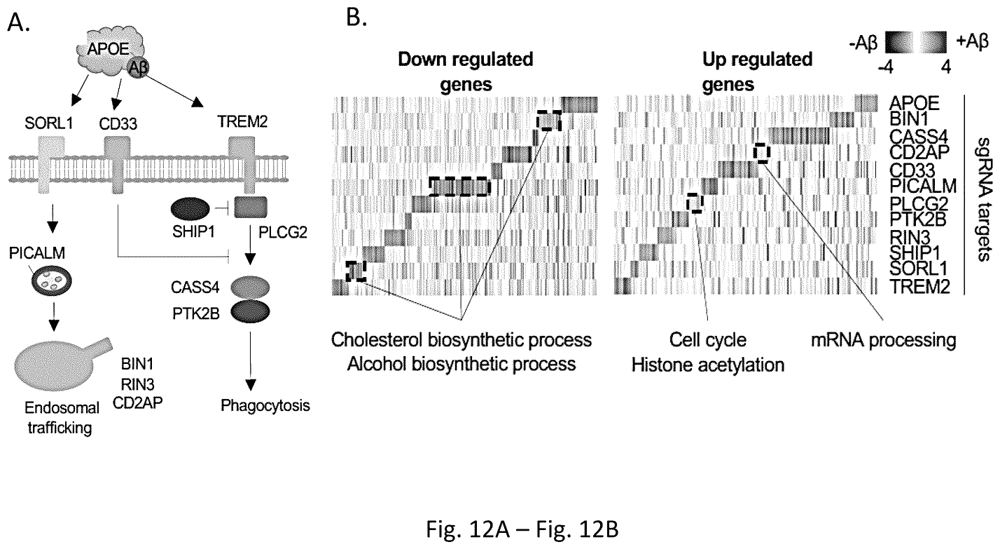

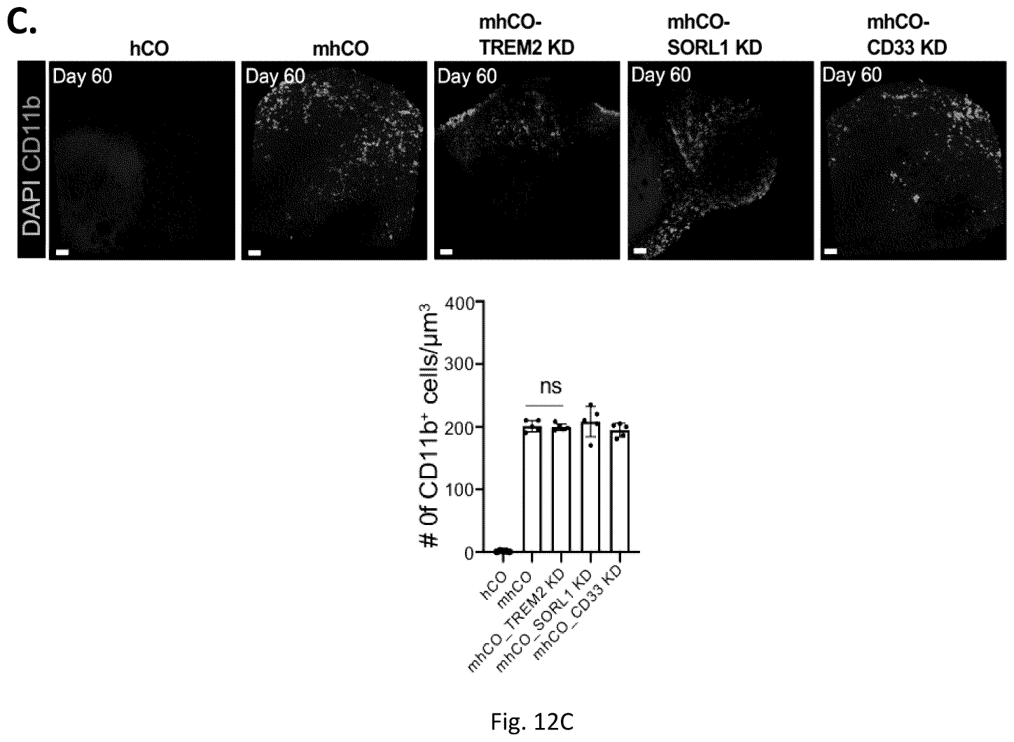

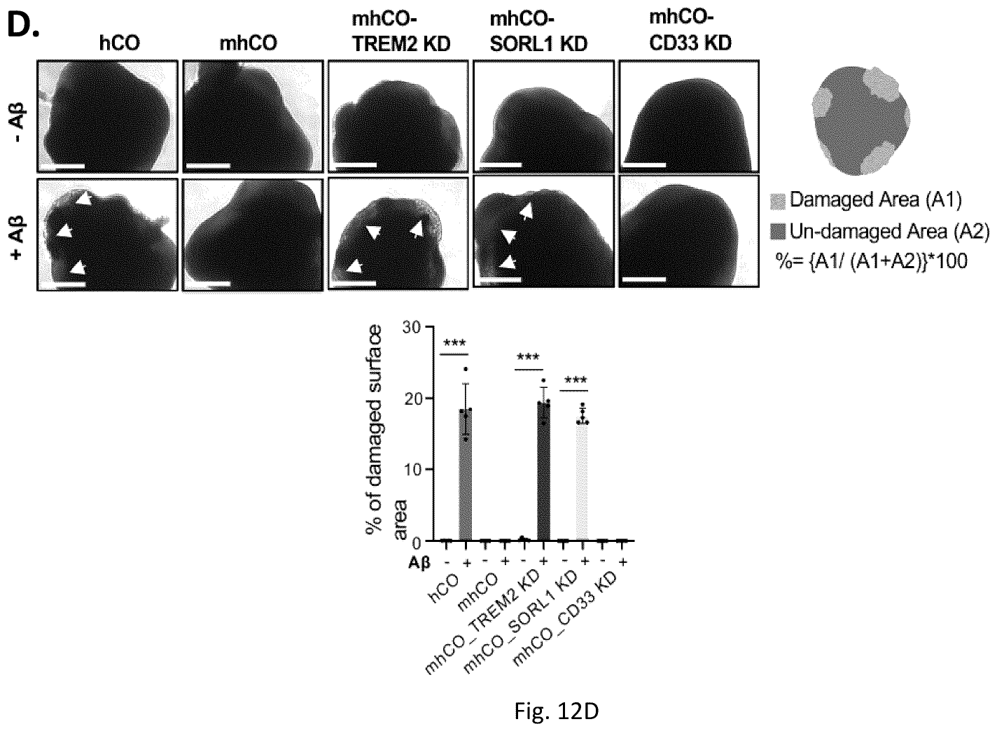

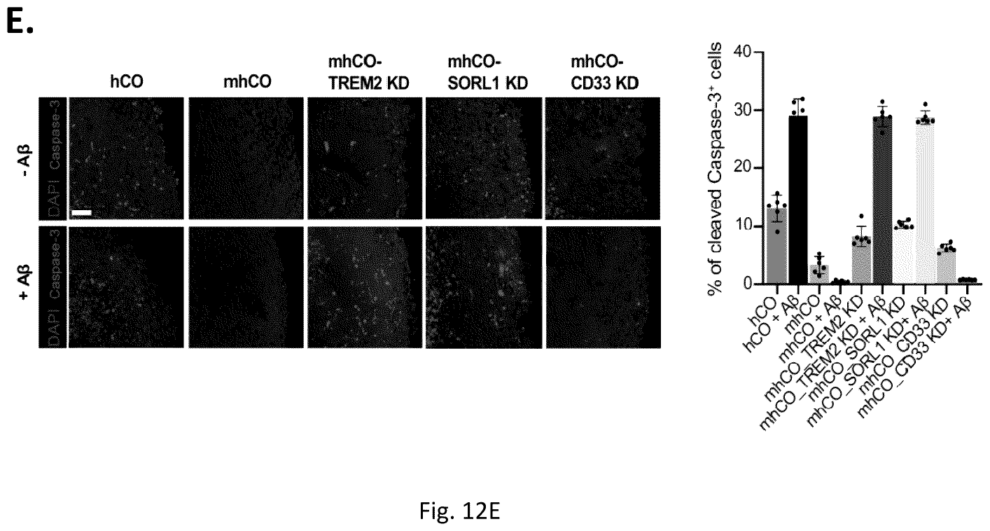

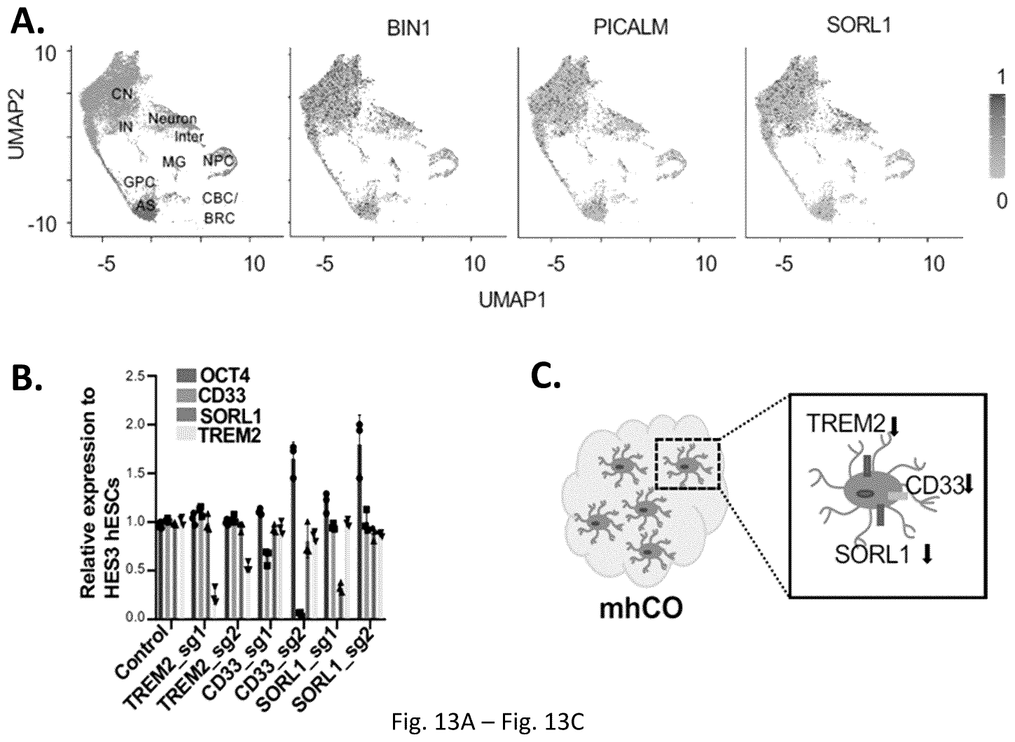

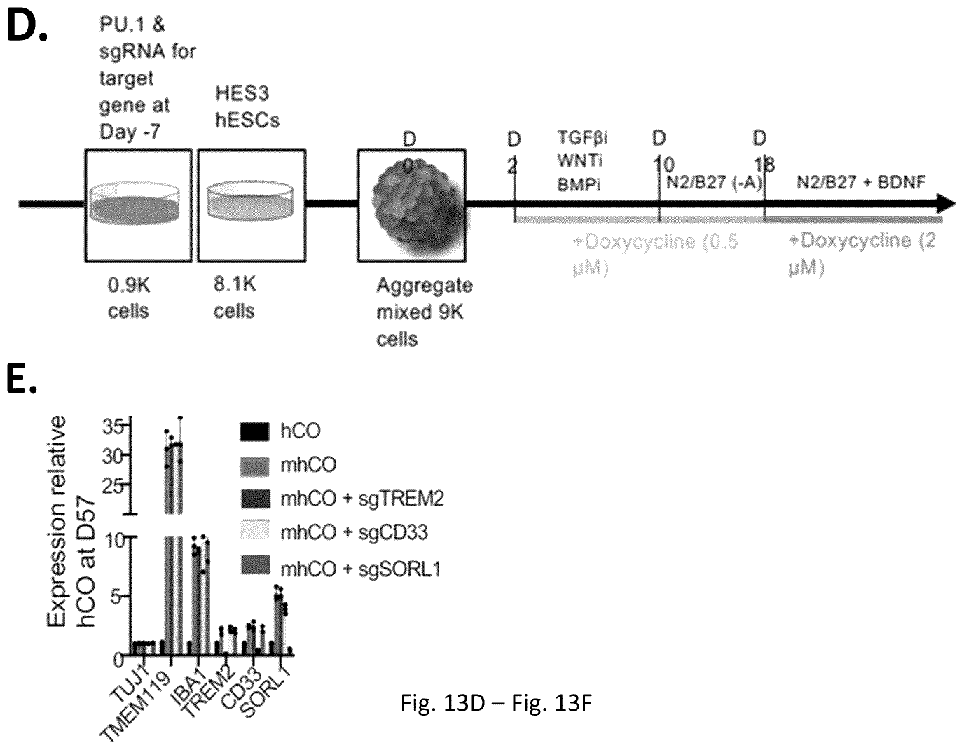

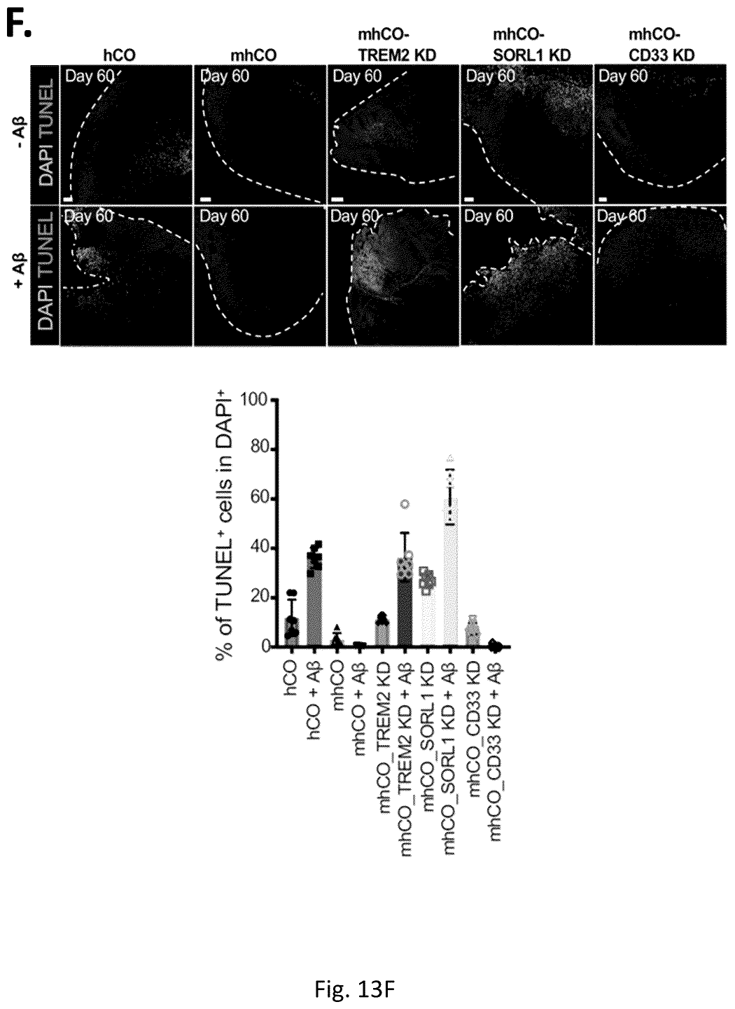

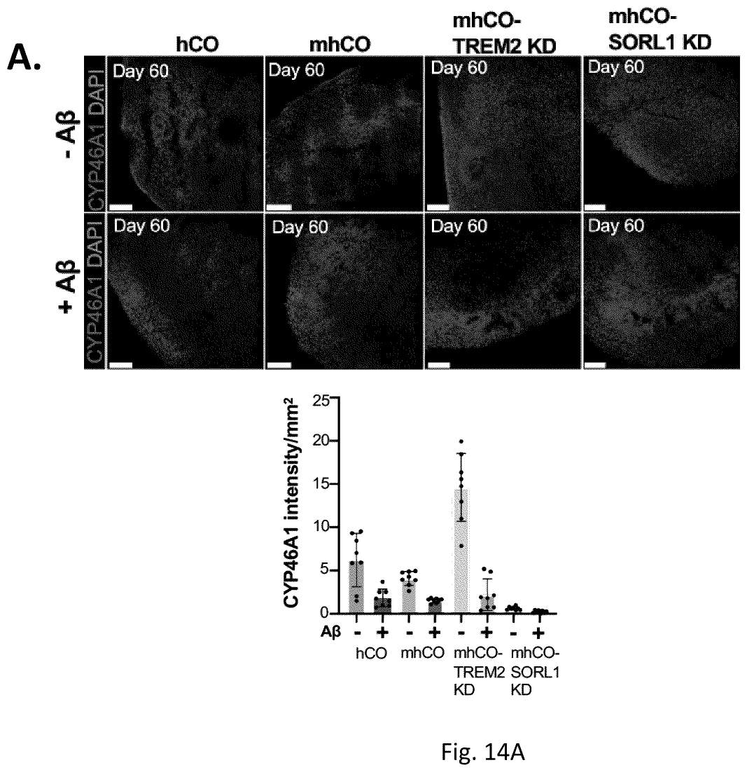

Previous genome-wide association studies (GWAS) and exome-sequencing have identified several microglia-associated and immune response genes for AD (Efthymiou et al., 2017, Mol Neurodegener, 12:43; Hollingworth et al., 2011, Nat Genet, 43:429-435). Using PU.1-induced microglia-like cells as a model system, the role of AD-linked genes in responding to Aβ treatment was explored (FIG. 12A). CRISPRi (CRISPR interference) coupled with CRISPR droplet sequencing (CROP-seq) format was used to suppress the expression of these genes and to implement high-throughput genetic perturbation at single-cell level (Datlinger et al., 2017, Nat Methods 14, 297-301). A gRNA library was designed for targeting 12 AD risk genes that are involved in endocytic trafficking, degradation, and phagocytosis of Aβ₁₋₄₂ (FIG. 12A and Table 3). After hESCs stably expressing dead Cas9 (dCas9) were transduced with gRNA library lentivirus and organoids generated, the function of AD-risk genes under Aβ₁₋₄₂-oligo treatment was analyzed via CROP-seq. By comparing transcriptome patterns with and without Aβ₁₋₄₂ treatment, a significant reduction of cholesterol metabolic genes by Aβ₁₋₄₂ treatment was identified in cells that are inhibited for endocytosis-related genes, PICALM, SORL1, and BIN1 (FIG. 12B and FIG. 13A). Previous GWAS studies also stressed that genes associated with endocytosis were linked with the development of late-onset AD and may lead to the perturbation of cholesterol homeostasis (Harold et al., 2009, Nat Genet, 41:1088-1093; Lambert et al., 2013, Nat Genet, 45:1452-1458). In addition, a low quantity of cholesterol promotes the formation of voltage-independent ion channel pores with Aβ₁₋₄₂ and results in disruption of Ca²⁺ homeostasis and membrane potential in neuron (Gao et al., 2020, Biochemistry, 59:992-998). This result suggests that pooled CRISPRi screening from mhCOs provides a valuable tool to better understand the role of microglia in AD.

TABLE 3
gRNA library used for the CROP-seq AD-linked genes screen.

| Gene Target | SEQ ID NO: | Sequence |
|---|---|---|
| TREM2-sg1 | 57 | GAAAGACGAGATCTTGCACA |
| TREM2-sg2 | 58 | CGCCTTCATAATTCACCCCA |
| CD33-sg1 | 59 | GGGGAGTTCTTGTCGTAGTA |
| CD33-sg2 | 60 | GACAAGAACTCCCCAGTTCA |
| SORL1-sg1 | 61 | CAGTAGCGTTCGCCCGAACA |
| SORL1-sg2 | 62 | CGCTGCACATTCTCTCCTGG |
| APOE4-sg1 | 63 | AGGACGTCCTTCACCTCCGC |
| APOE4-sg2 | 64 | AGGGTCCCAGCTCTTTCTAG |
| PICALM-sg1 | 65 | TTAGAATGGCAGCAACGTGT |
| SHIP1-sg1 | 66 | GAGCCGGTCATTCCACCCAG |
| CD2AP-sg1 | 67 | AGTGCTAAGGAAGAGGCGAG |
| RIN3-sg1 | 68 | ATCATGCCGCCGGCAGCTCC |
| BIN1-sg1 | 69 | AAGGCAGCTTATTGTCCGGA |
| PLCG2-sg1 | 70 | GAAGCAGAAGTAGCGAGCGC |
| CASS4-sg1 | 71 | CAGGCATTGAGACGTGAGTG |
| PTK2B-sg1 | 72 | AGGTAGGTGTGCAACGGCTC |

In addition to pooled CRISPRi screening, the role of 3 AD-linked microglia genes (TREM2, CD33, and SORL1) was further explored by generating mhCOs with targeted suppression of each gene (FIG. 13B, FIG. 13C and FIG. 13D). Two gRNAs for each target were tested for the efficacy of CRISPRi in hESCs (Table 3), and one with higher efficiency of gene repression was used to suppress the given gene in PU.1-induced MG in mhCOs (FIG. 13D). The expression of target genes was dramatically down-regulated in mhCOs by the given gRNA (FIG. 13B). Whether the suppression of the AD-associated genes affected the generation of mhCOs was assessed. Regardless of the repression of AD-associated genes, mhCOs showed a similar expression of the microglia markers TMEM119 and IBA1 (FIG. 13E) and presented with a similar number of CD11b⁺ microglia-like cells (FIG. 12C), suggesting a successful formation of cortical organoids with microglia. Even though the suppression of AD-associated genes in mhCOs did not alter the density of CD11b⁺ microglia like-cells, the distribution of microglia-like cells in mhCOs is perturbed (FIG. 12C). For instance, in line with previous study (Parhizkar et al., 2019, Nat Neurosci, 22:191-204), TREM2 suppression in microglia-like cells could limit their ability to cluster around the Aβ peptides. Thus, the results suggest that downregulation of AD-associated genes in immune cells has no effects on microglia formation but their functions in mhCOs.

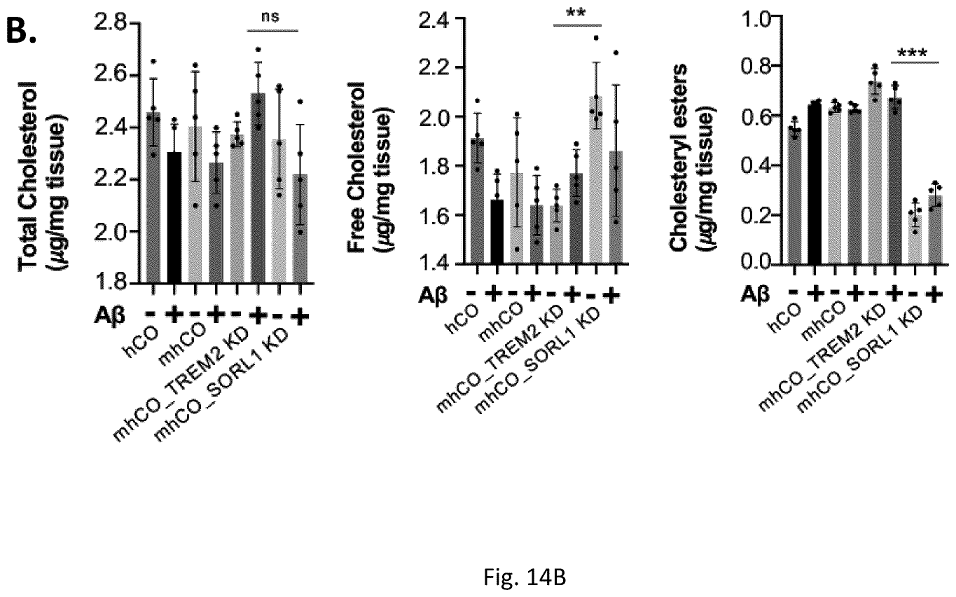

Next, how the suppression of the AD-associated genes in PU.1-induced MG affects the mhCOs when treated with Aβ. Aβ₁₋₄₂-oligo treatment resulted in a dramatic morphological change, particularly on the surface of TREM2- or SORL1-suppressed mhCOs was examined. However, the suppression of CD33 did not have any impact (FIG. 12D). Importantly, a dramatic increase in cell death was observed in TREM2- or SORL1-suppressed mhCOs to an extent as control hCOs treated with Aβ₁₋₄₂-oligo, characterized by cleaved Caspase-3 and TUNEL staining (FIG. 12E and FIG. 13F). The suppression of CD33 did not affect the microglial immune response to Aβ (FIG. 12E and FIG. 13F). In line with previous mice studies (Griciuc et al., 2019, Neuron, 103:820-835.e7; Griciuc et al., 2013, Neuron 78, 631-643), CD33 acts upstream of TREM2 and inhibits the Aβ oligo uptake in microglia. Moreover, the dysregulation of cholesterol metabolism via the SORL1 gene, as indicated in the CRISPRi screen (FIG. 12A), was examined by using SORL1-suppressed mhCOs. Since cholesterol 24-hydroxylase (CYP46A1) takes part in the removal of cholesterol in the brain (Lund et al., 1999, Proc Natl Acad Sci USA, 96:7238-7243), staining for CYP46A1 in organoids was performed (FIG. 14A). Notably, SORL1-suppressed mhCOs demonstrated the significantly reduced CYP46A1 expression compared to hCOs and mhCOs with and without Aβ-treatment (FIG. 14A). Lastly, the cholesterol turnover in mhCO variants was examined. Control and mhCO variants possessed similar total cholesterol levels with and without Aβ treatment (FIG. 14B). Interestingly, SORL1 suppressed mhCOs exhibited significantly increased free cholesterol levels and decreased cholesteryl esters than other organoids (FIG. 14B). Thus, SORL1 suppression leads to decreased CYP461 expression, in turn, decreased cholesterol turnover rate, consistent with previous study mice study (Xie et al., 2003, J Lipid Res, 44:1780-1789). Overall, these data suggest that the PU.1-induced microglia-like cells in mhCOs are an excellent model to investigate the function of AD-associated genes in responding to Aβ.

Inducible Pu. 1 cassette
(SEQ ID NO: 75)
ATGGAAGGGTTTCCCCTCGTCCCCCCTCCATCAGAAGACCTGGTGCCCT
ATGACACGGATCTATACCAACGCCAAACGCACGAGTATTACCCCTATCT
CAGCAGTGATGGGGAGAGCCATAGCGACCATTACTGGGACTTCCACCCC
CACCACGTGCACAGCGAGTTCGAGAGCTTCGCCGAGAACAACTTCACGG
AGCTCCAGAGCGTGCAGCCCCCGCAGCTGCAGCAGCTCTACCGCCACAT
GGAGCTGGAGCAGATGCACGTCCTCGATACCCCCATGGTGCCACCCCAT
CCCAGTCTTGGCCACCAGGTCTCCTACCTGCCCCGGATGTGCCTCCAGT
ACCCATCCCTGTCCCCAGCCCAGCCCAGCTCAGATGAGGAGGAGGGCGA
GCGGCAGAGCCCCCCACTGGAGGTGTCTGACGGCGAGGCGGATGGCCTG
GAGCCCGGGCCTGGGCTCCTGCCTGGGGAGACAGGCAGCAAGAAGAAGA
TCCGCCTGTACCAGTTCCTGTTGGACCTGCTCCGCAGCGGCGACATGAA
GGACAGCATCTGGTGGGTGGACAAGGACAAGGGCACCTTCCAGTTCTCG
TCCAAGCACAAGGAGGCGCTGGCGCACCGCTGGGGCATCCAGAAGGGCA
ACCGCAAGAAGATGACCTACCAGAAGATGGCGCGCGCGCTGCGCAACTA
CGGCAAGACGGGCGAGGTCAAGAAGGTGAAGAAGAAGCTCACCTACCAG
TTCAGCGGCGAAGTGCTGGGCCGCGGGGGCCTGGCCGAGCGGCGCCACC
CGCCCCACTGA

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for preparing a microglial cell comprising cortical organoid culture, wherein the method comprises:
a) co-culturing human epithelial stem cells containing an inducible Pu.1 transcription factor gene (Pu.1-hESCs) with human epithelial stem cells without the inducible Pu.1 transcription factor gene (hESCs) in neural induction media for at least 8 days;
b) transferring the cells to a spinning human cortical organoid culture medium lacking vitamin A for at least 4 days;
c) transferring the cells to a human cortical organoid culture medium with vitamin A; and
d) inducing Pu.1 expression.

2. The method of claim 1, wherein the Pu.1-hESCs and hESCs are co-cultured at a ratio of 1:9.

3. The method of claim 1, wherein the neural induction media comprises DMEM-F12 with 15% (v/v) KSR, 5% (v/v) heat-inactivated FBS, 1% (v/v) Glutamax, 1% (v/v) MEM-NEAA, 100 μM β-Mercaptoethanol; supplemented with 10 μM SB-431542, 100 nM LDN-193189, 2 μM XAV-939 and 50 μM Y27632.

4. The method of claim 1, wherein Pu.1 is inducible by doxycycline, and further wherein doxycycline is added to the neural induction media in a concentration of 0.5 μM dox beginning on day 2.

5. The method of claim 1, wherein FBS is removed from the neural induction medium beginning on day 2.

6. The method of claim 1, wherein Y27632 is removed from the neural induction medium beginning on day 4.

7. The method of claim 1, wherein the spinning human cortical organoid culture medium lacking vitamin A comprises a 1:1 mixture of DMEM-F12 and Neurobasal media, 0.5% (v/v) N2 supplement, 1% (v/v) B27 supplement without vitamin A, 0.5% (v/v) MEM-NEAA, 1% (v/v) Glutamax, 50 μM β-Mercaptoethanol, 1% (v/v) Penicillin/Streptomycin and 0.025% Insulin.

8. The method of claim 1, wherein the human cortical organoid culture medium with vitamin A comprises a 1:1 mixture of DMEM-F12 and Neurobasal media, 0.5% (v/v) N2 supplement, 1% (v/v) B27 supplement, 0.5% (v/v) MEM-NEAA, 1% (v/v) Glutamax, 50 μM β-Mercaptoethanol, 1% (v/v) Penicillin/Streptomycin and 0.025% Insulin.

9. The method of claim 1, wherein Pu.1 is inducible by doxycycline, and further wherein doxycycline is added to the human cortical organoid culture medium with vitamin A in a concentration of 2 μM dox.

10. A microglial cell comprising cortical organoid culture obtained by the method of claim 1.

11. An assay system comprising a microglial cell comprising cortical organoid culture of claim 10.

12. A method of assaying a target agent comprising contacting a microglial cell comprising cortical organoid culture of claim 10 with the target agent.

13. A method for identifying a therapeutic agent, wherein the method comprises:
contacting a microglial cell comprising cortical organoid culture of claim 10 with one or more candidate agents,
detecting the presence or absence of one or more change in the microglial cell comprising cortical organoid culture that is indicative of therapeutic efficacy, and
identifying the candidate agent as a therapeutic agent if the presence or absence of one or more of said changes in the microglial cell comprising cortical organoid culture is detected.

14. The method of claim 13, wherein the said change in the microglial cell comprising cortical organoid co-culture is selected from the group consisting of a change in cell viability, organoid size, morphology, quantification of epithelial subsets, cell proliferation, transcriptome, protein levels or post-translational modifications of proteins, metabolism, production of soluble factors and any combination thereof of the microglial cell comprising cortical organoid cells as compared to a comparator control.

15. The method of claim 13, wherein the therapeutic agent is suitable for the treatment of a neurodevelopmental or neurodegenerative disease or disorder.

16. The method of claim 13, wherein the disease or disorder is selected from the group consisting of autism, schizophrenia, Alzheimer's disease (AD) or another dementia, Parkinson's disease (PD) or a PD-related disorder, frontotemporal dementia (FTD), amyotrophic lateral sclerosis (ALS), Huntington's disease (HD), spinocerebellar ataxias (SCAs), prion disease, spinal muscular atrophy (SMA), lewy body dementia (LBD), multisystem atrophy, primary progressive aphasia, multiple sclerosis (MS), ischemic stroke, traumatic brain injury, HIV-associated dementia, or another neurodegenerative, neurological or psychiatric disease or disorder.

17. A kit comprising at least one component for use in a method of claim 1.

18. The kit of claim 17, comprising Pu.1ⁱ-hESCs.

19. A kit comprising at least one microglial cell comprising cortical organoid culture obtained by the method of claim 1.


SEQUENCE LISTING (75 sequences)

SEQ ID NO: 1 - DNA, 21 bp, synthetic construct
  source 1..21: mol_type = other DNA
cgaccattactgggacttcca

SEQ ID NO: 2 - DNA, 22 bp, synthetic construct
  source 1..22: mol_type = other DNA
ggagctccgtgaagttgttctc

SEQ ID NO: 3 - DNA, 19 bp, synthetic construct
  source 1..19: mol_type = other DNA
gtccctgaaacgaatgctg

SEQ ID NO: 4 - DNA, 22 bp, synthetic construct
  source 1..22: mol_type = other DNA
ccttttctctcgctttttcctc

SEQ ID NO: 5 - DNA, 22 bp, synthetic construct
  source 1..22: mol_type = other DNA
cgtctccatcatgtataacctc

SEQ ID NO: 6 - DNA, 20 bp, synthetic construct
  source 1..20: mol_type = other DNA
gtaaagtcgccattgtctcc

SEQ ID NO: 7 - DNA, 19 bp, synthetic construct
  source 1..19: mol_type = other DNA
caacagcaagaagcccaag

SEQ ID NO: 8 - DNA, 21 bp, synthetic construct
  source 1..21: mol_type = other DNA
cgatgaagaagaaggcggtag

SEQ ID NO: 9 - DNA, 19 bp, synthetic construct
  source 1..19: mol_type = other DNA
tccagaacaaccctaaccc

SEQ ID NO: 10 - DNA, 20 bp, synthetic construct
  source 1..20: mol_type = other DNA
cgccaaacttttctccatcc

SEQ ID NO: 11 - DNA, 20 bp, synthetic construct
  source 1..20: mol_type = other DNA
gcctcctcatccttctgttg

SEQ ID NO: 12 - DNA, 21 bp, synthetic construct
  source 1..21: mol_type = other DNA
aagaagtccactatcccatcc

SEQ ID NO: 13 - DNA, 21 bp, synthetic construct
  source 1..21: mol_type = other DNA
acatccaaccccaaaaatctc

SEQ ID NO: 14 - DNA, 20 bp, synthetic construct
  source 1..20: mol_type = other DNA
tttacctacacccctcgttc

SEQ ID NO: 15 - DNA, 21 bp, synthetic construct
  source 1..21: mol_type = other DNA
tcacaacctctctacctcaac

SEQ ID NO: 16 - DNA, 19 bp, synthetic construct
  source 1..19: mol_type = other DNA
tgcattctgagaagccaac

SEQ ID NO: 17 - DNA, 19 bp, synthetic construct
  source 1..19: mol_type = other DNA
ccaaccgccacaaaacttc

SEQ ID NO: 18 - DNA, 19 bp, synthetic construct
  source 1..19: mol_type = other DNA
tatgttcccaaccagtccc

SEQ ID NO: 19 - DNA, 20 bp, synthetic construct
  source 1..20: mol_type = other DNA
ccatcctcacaccccttatc

SEQ ID NO: 20 - DNA, 20 bp, synthetic construct
  source 1..20: mol_type = other DNA
tcttgccttagccctatgtc

SEQ ID NO: 21 - DNA, 20 bp, synthetic construct
  source 1..20: mol_type = other DNA
caccaagctcgcaatccctc

SEQ ID NO: 22 - DNA, 20 bp, synthetic construct
  source 1..20: mol_type = other DNA
ctctaccacccggaagaaca

SEQ ID NO: 23 - DNA, 20 bp, synthetic construct
  source 1..20: mol_type = other DNA
accatccgcaaccagatcaa

SEQ ID NO: 24 - DNA, 18 bp, synthetic construct
  source 1..18: mol_type = other DNA
gctcctcgctgccgtaca

SEQ ID NO: 25 - DNA, 19 bp, synthetic construct
  source 1..19: mol_type = other DNA
ccgcatccctcacatgaaa

SEQ ID NO: 26 - DNA, 20 bp, synthetic construct
  source 1..20: mol_type = other DNA
tcccagaccaccagcttgtc

SEQ ID NO: 27 - DNA, 21 bp, synthetic construct
  source 1..21: mol_type = other DNA
cactttcttccatcccatacc

SEQ ID NO: 28 - DNA, 20 bp, synthetic construct
  source 1..20: mol_type = other DNA
tgaaccattatccctcctcc

SEQ ID NO: 29 - DNA, 19 bp, synthetic construct
  source 1..19: mol_type = other DNA
aaactgccctaccaccatc

SEQ ID NO: 30 - DNA, 21 bp, synthetic construct
  source 1..21: mol_type = other DNA
actttcatcactgttgtctcc

SEQ ID NO: 31 - DNA, 21 bp, synthetic construct
  source 1..21: mol_type = other DNA
cacccacttccatccttctcc

SEQ ID NO: 32 - DNA, 20 bp, synthetic construct
  source 1..20: mol_type = other DNA
tccagttcactgggtggatg

SEQ ID NO: 33 - DNA, 19 bp, synthetic construct
  source 1..19: mol_type = other DNA
caccacatccaccatttcc

SEQ ID NO: 34 - DNA, 19 bp, synthetic construct
  source 1..19: mol_type = other DNA
aagaactgtccactcgcac

SEQ ID NO: 35 - DNA, 21 bp, synthetic construct
  source 1..21: mol_type = other DNA
aagagggacagacacaaaaag

SEQ ID NO: 36 - DNA, 22 bp, synthetic construct
  source 1..22: mol_type = other DNA
cagcaaaatgccatacacaaag

SEQ ID NO: 37 - DNA, 20 bp, synthetic construct
  source 1..20: mol_type = other DNA
acacaccacctacccctttc

SEQ ID NO: 38 - DNA, 22 bp, synthetic construct
  source 1..22: mol_type = other DNA
tctcatctacatccactgcttc

SEQ ID NO: 39 - DNA, 19 bp, synthetic construct
  source 1..19: mol_type = other DNA
atggatgtggataggcgag

SEQ ID NO: 40 - DNA, 20 bp, synthetic construct
  source 1..20: mol_type = other DNA
tgcagcgataatcagaggag

SEQ ID NO: 41 - DNA, 22 bp, synthetic construct
  source 1..22: mol_type = other DNA
aaacaacccctcctctaaaaac

SEQ ID NO: 42 - DNA, 21 bp, synthetic construct
  source 1..21: mol_type = other DNA
tttgacctttgctctctgaac

SEQ ID NO: 43 - DNA, 20 bp, synthetic construct
  source 1..20: mol_type = other DNA
cgagtccgtgaagaatcaag

SEQ ID NO: 44 - DNA, 20 bp, synthetic construct
  source 1..20: mol_type = other DNA
gggtagaggtgagacagtag

SEQ ID NO: 45 - DNA, 19 bp, synthetic construct
  source 1..19: mol_type = other DNA
gcacgactaaaagggctac

SEQ ID NO: 46 - DNA, 20 bp, synthetic construct
  source 1..20: mol_type = other DNA
aggagggagaatggaggaag

SEQ ID NO: 47 - DNA, 22 bp, synthetic construct
  source 1..22: mol_type = other DNA
ttctctatcccacccactaatc

SEQ ID NO: 48 - DNA, 19 bp, synthetic construct
  source 1..19: mol_type = other DNA
atcttctgcccccaacatc

SEQ ID NO: 49 - DNA, 21 bp, synthetic construct
  source 1..21: mol_type = other DNA
aattccattatcctcgctgtc

SEQ ID NO: 50 - DNA, 21 bp, synthetic construct
  source 1..21: mol_type = other DNA
acccttcccatcaatacatcc

SEQ ID NO: 51 - DNA, 20 bp, synthetic construct
  source 1..20: mol_type = other DNA
ccatcaccaagcagaacgag

SEQ ID NO: 52 - DNA, 20 bp, synthetic construct
  source 1..20: mol_type = other DNA
acaccgcgtagaagtacgag

SEQ ID NO: 53 - DNA, 20 bp, synthetic construct
  source 1..20: mol_type = other DNA
cctcacttcactgcactgta

SEQ ID NO: 54 - DNA, 20 bp, synthetic construct
  source 1..20: mol_type = other DNA
caggttttctttccctagct

SEQ ID NO: 55 - DNA, 19 bp, synthetic construct
  source 1..19: mol_type = other DNA
gccgctacctgacggtggc

SEQ ID NO: 56 - DNA, 20 bp, synthetic construct
  source 1..20: mol_type = other DNA
gggcgggatgtcacacacgg

SEQ ID NO: 57 - DNA, 20 bp, synthetic construct
  source 1..20: mol_type = other DNA
gaaagacgagatcttgcaca

SEQ ID NO: 58 - DNA, 20 bp, synthetic construct
  source 1..20: mol_type = other DNA
cgccttcataattcacccca

SEQ ID NO: 59 - DNA, 20 bp, synthetic construct
  source 1..20: mol_type = other DNA
ggggagttcttgtcgtagta

SEQ ID NO: 60 - DNA, 20 bp, synthetic construct
  source 1..20: mol_type = other DNA
gacaagaactccccagttca

SEQ ID NO: 61 - DNA, 20 bp, synthetic construct
  source 1..20: mol_type = other DNA
cagtagcgttcgcccgaaca

SEQ ID NO: 62 - DNA, 20 bp, synthetic construct
  source 1..20: mol_type = other DNA
cgctgcacattctctcctgg

SEQ ID NO: 63 - DNA, 20 bp, synthetic construct
  source 1..20: mol_type = other DNA
aggacgtccttcacctccgc

SEQ ID NO: 64 - DNA, 20 bp, synthetic construct
  source 1..20: mol_type = other DNA
agggtcccagctctttctag

SEQ ID NO: 65 - DNA, 20 bp, synthetic construct
  source 1..20: mol_type = other DNA
ttagaatggcagcaacgtgt

SEQ ID NO: 66 - DNA, 20 bp, synthetic construct
  source 1..20: mol_type = other DNA
gagccggtcattccacccag

SEQ ID NO: 67 - DNA, 20 bp, synthetic construct
  source 1..20: mol_type = other DNA
agtgctaaggaagaggcgag

SEQ ID NO: 68 - DNA, 20 bp, synthetic construct
  source 1..20: mol_type = other DNA
atcatgccgccggcagctcc

SEQ ID NO: 69 - DNA, 20 bp, synthetic construct
  source 1..20: mol_type = other DNA
aaggcagcttattgtccgga

SEQ ID NO: 70 - DNA, 20 bp, synthetic construct
  source 1..20: mol_type = other DNA
gaagcagaagtagcgagcgc

SEQ ID NO: 71 - DNA, 20 bp, synthetic construct
  source 1..20: mol_type = other DNA
caggcattgagacgtgagtg

SEQ ID NO: 72 - DNA, 20 bp, synthetic construct
  source 1..20: mol_type = other DNA
aggtaggtgtgcaacggctc

SEQ ID NO: 73 - DNA, 29 bp, synthetic construct
  source 1..29: mol_type = other DNA
tttggatccaaggcccaccatggaagggt

SEQ ID NO: 74 - DNA, 31 bp, synthetic construct
  source 1..31: mol_type = other DNA
tttgtttaaactcattactaagcgtagtctg

SEQ ID NO: 75 - DNA, 795 bp, synthetic construct
  source 1..795: mol_type = other DNA
atggaagggtttcccctcgtcccccctccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactga